(19) United States
(12) Patent Application Publication
(10) Pub. No.: US 2024/0115610 A1
(43) Pub. Date: Apr. 11, 2024

(54) GENETICALLY ENGINEERED DIAGNOSTIC CELLS AND ANTIGEN TESTS

(71) Applicant: SRI International, Menlo Park, CA (US)

(72) Inventors: Parijat BHATNAGAR, Belmont, CA (US); Marvin A. SSEMADAALI, Menlo Park, CA (US)

(73) Assignee: SRI International, Menlo Park, CA (US)

(21) Appl. No.: 18/333,778

(22) Filed: Jun. 13, 2023

Related U.S. Application Data
(62) Division of application No. 17/586,749, filed on Jan. 27, 2022, now Pat. No. 11,672,829.
Application No. 17/586,749 is a continuation-in-part of application No. 15/263,078, filed on Sep. 12, 2016.
(60) Provisional application No. 62/249,986, filed on Nov. 3, 2015.
(60) Provisional application No. 62/216,538, filed on Sep. 10, 2015.
(60) Provisional application No. 63/255,380, filed on Oct. 13, 2021.
(60) Provisional application No. 63/222,784, filed on Jul. 16, 2021.
(60) Provisional application No. 63/142,315, filed on Jan. 27, 2021.

(51) Int. Cl. A61K 35/17; A61P 31/12; C07K 14/725; C07K 16/28; C12N 5/0783

(52) U.S. Cl. CPC A61K 35/17; A61P 31/12; C07K 14/7051; C07K 16/2803; C12N 5/0636; A61K 38/00

(57) ABSTRACT

An example antigen test device comprises a genetically engineered diagnostic cell comprising an exogenous polynucleotide sequence including a receptor element that encodes a chimeric antigen receptor (CAR) comprising an extracellular antigen binding domain operably linked to a transmembrane domain, and an intracellular signaling domain that recognizes an antigen on a surface of a pathogen-infected cell from a sample or on a surface of a virus particle associated with a pathogen from the sample, an actuator element that encodes a transcription factor binding site, and an effector element that encodes a detectable reporter protein, wherein, in response to the antigen binding domain of the CAR binding to the antigen, the genetically engineered diagnostic cell is configured to activate and to synthesize the detectable reporter protein.

### CROSS-REFERENCE TO RELATED APPLICATIONS

This application is a Divisional of U.S. patent application Ser. No. 17/586,749, filed on Jan. 27, 2022, which claims benefit to U.S. Provisional Patent Application No. 63/255,380, filed Oct. 13, 2021, U.S. Provisional Patent Application No. 63/222,784, filed Jul. 16, 2021, and U.S. Provisional Patent Application No. 63/142,315, filed Jan. 27, 2021, and which is a continuation in part of U.S. patent application Ser. No. 15/263,078, filed on Sep. 12, 2016, and which claims benefit to U.S. Provisional Patent Application No. 62/249,986, filed on Nov. 3, 2015 and U.S. Provisional Patent Application No. 62/216,538, filed on Sep. 10, 2015, which are each incorporated herein by reference in their entireties.

### GOVERNMENT RIGHTS

This invention was made with Government support under contract no. D19AP00024 awarded by the Defense Advanced Research Projects Agency and with Government support under grant number 1DP2EB024245-01 awarded by National Institutes of Health, under contract no. DP2EB0242454 awarded by the National Institute of Biomedical Imaging and Bioengineering of the National Institutes of Health and under grant number 7R21CA193064-02 awarded by the National Institutes of Health. The government has certain rights to this invention.

### INCORPORATION-BY-REFERENCE OF MATERIAL SUBMITTED ELECTRONICALLY

The contents of the electronic sequence listing (S1647137103_Sequence_Listing.xml; Size: 365,216 bytes; and Date of Creation: Jun. 13, 2023) is herein incorporated by reference in its entirety.

### BACKGROUND

Vaccination is an effective approach for preventing epidemic outbreaks. However, the process to develop vaccines is lengthy and resource-intensive, requiring antigen identification and determining safe and effective administration. Accordingly, there is an unmet need for a platform treatment that can be rapidly deployed in the event of an outbreak or biological attack, such as with Severe Acute Respiratory Syndrome Coronavirus 2 (SARS-CoV-2).

### BRIEF DESCRIPTION OF THE DRAWINGS

The patent application file contains at least one drawing executed in color. Copies of this patent application publication with color drawings will be provided by the Office upon request and payment of the necessary fee.

Various example embodiments can be more completely understood in consideration of the following detailed description in connection with the accompanying drawings, in which:

FIG. 1 illustrates an example of a genetically engineered effector cell that is specific to SARS-CoV-2, in accordance with the present disclosure.

FIG. 2 illustrates an example of a genetically engineered effector cell and a sequence of events triggered when in a diseased environment, in accordance with the present disclosure.

FIG. 3 illustrates an example of a population of genetically engineered effector cells in a diseased environment, in accordance with the present disclosure.

FIG. 4 illustrates an example method of contacting a plurality of cells with a volume of a genetically engineered effector cell, in accordance with the present disclosure.

FIGS. 5A-5C illustrate an example of a genetically engineered diagnostic cell and a sequence of events triggered for providing an antigen test, in accordance with the present disclosure.

FIGS. 6A-6D illustrate an example of a genetically engineered reporter cell and a sequence of events triggered for providing a serology test, in accordance with the present disclosure.

FIGS. 7A-7F illustrate plots characterizing genetically engineered effector cell function with specificity against SARS-CoV-2 antigens, in accordance with the present disclosure.

FIG. 8 illustrates plots characterizing genetically engineered effector cell function with specificity against different pathogens, in accordance with the present disclosure.

FIGS. 9A-9D illustrate plots characterizing genetically engineered effector cell function, in accordance with the present disclosure.

FIGS. 10A-10D illustrate plots characterizing therapeutic activity of example genetically engineered effector cells, in accordance with the present disclosure.

FIG. 11 illustrates plots characterizing IFN signaling of example genetically engineered effector cells, in accordance with the present disclosure.

FIGS. 12A-12B illustrate plots characterizing activation and growth of example genetically engineered effector cells, in accordance with the present disclosure.

FIGS. 13A-13D illustrate plots characterizing activation of example genetically engineered effector cells by SARS-CoV-2-infected cells, in accordance with the present disclosure.

FIGS. 14A-14B illustrate plots characterizing activation and growth curves of example genetically engineered effector cells, in accordance with the present disclosure.

FIGS. 15A-15D illustrate plots characterizing prophylactic and therapeutic activating of example genetically engineered effector cells, in accordance with the present disclosure.

FIGS. 16A-16D illustrate plots characterizing activation of example genetically engineered effector cells, in accordance with the present disclosure.

FIG. 17 illustrates plots characterizing IFN signaling of example genetically engineered effector cells in response to SARS-CoV-2-infected cells, in accordance with the present disclosure.

FIGS. 18A-18D illustrate plots characterizing activation of example genetically engineered diagnostic cells in response to antigen-presenting target cells, in accordance with the present disclosure.

FIGS. 19A-19F show example data results of implementing an example diagnostic cell with infectious SARS-CoV-2 virus particles, in accordance with the present disclosure.

FIGS. 20A-20B illustrate results of detecting infection in mouse oropharyngeal swabs using an example diagnostic cell, in accordance with the present disclosure.

FIGS. 21A-21D illustrate example results of assessing VHH-72 diagnostic cell activation by genetically engineered target SARS-CoV-1-Sgp-cells, in accordance with the present disclosure.

FIG. 22 illustrates example results of screening different diagnostic cells with specificity towards SARS-CoV-2 and SARS-CoV-1, in accordance with the present disclosure.

FIG. 23 illustrates detection of heat-inactivated target cells by example VHH-Tyl diagnostic cells, in accordance with the present disclosure.

FIG. 24 illustrates example assessment of different diagnostic cells generated with specificity to other emerging viruses, in accordance with the present disclosure.

FIGS. 25A-25D illustrate plots characterizing assessment of example reporter cell complexes, in accordance with the present disclosure.

FIGS. 26A-26D illustrate plots characterizing assessment of use of an example reporter cell complex in a serology test using a commercial serum panel, in accordance with the present disclosure.

FIGS. 27A-27B illustrate plots characterizing clinical validation of an example reporter cell complex for detecting IgG antibodies specific to SARS-CoV-2, in accordance with the present disclosure.

FIG. 28 illustrates a plot characterizing sensitivity of an example reporter cell complex using different sera dilutions, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

In some embodiments, a cell can be engineered to express genetic elements including transmembrane receptor(s) that autonomously regulate the intracellular transcriptional machinery. The genetic elements of the cell can be modular and/or a cell can include multiple genetic elements to yield an engineered effector cell having the capacity to serve as a vector for a variety of in vitro, ex vivo, and in vivo applications. Such cells can be modular in that parts can be conserved, and parts can be changed for different applications. The genetically engineered effectors cells can be used for therapeutics and treatment methods that self-regulate the therapeutic response upon stimulation by the disease cells and that are applicable to a variety of cell-based diseases, including cancers, emerging pathogens, and others that evade the immune system or involve its malfunction. In other examples, the genetically engineered effector cells can be used in diagnostics, such as antigen or serology test for different pathogens or molecules. Multiple types of such genetically engineered cells provide a robust, reproducible cellular system to therapeutically target complex diseases in vivo. In various embodiments, the cell-based platform is useful for neutralizing viral threats and/or detecting infections or immunity. For example, in the wake of growing number of virus pandemics, the disclosed cell-based platform can be activated to produce antiviral proteins upon interaction with virally infected target cells, such SARS-CoV-2-infected cells.

COVID-19 morbidities and mortalities have continued to devastate global economies. Vaccines and other control measures have helped reduce its new infections, but there is few or no clinically approved therapies to treat the disease. Expression of both innate Type-I and Type-III interferons (IFNs) results in an anti-viral state within a host (e.g., a subject) to clear off the infection. However, a diminished IFN response in COVID-19 infections, that leads to progression of severe clinical symptoms, has been reported. In addition, although use of exogenous IFNs has shown therapeutic benefits, excessive or delayed induction have also been linked to poor clinical outcomes.

Embodiments of the present disclosure include cells and cell lines that are genetically engineered with chimeric antigen receptors (CARs) to specifically detect (e.g., bind) antigens expressed on the surface of SARs-CoV-2 infected cells. In response to the CAR binding to the antigens of SARs-CoV-2 infected cells, antiviral proteins (AVP) can be expressed. This AVP-producing cell is specific and its expression of the AVP is directly proportional to the number of SARs-CoV-2 infected cells. The cells are transformed into an in vivo living vector (antigen specific biofactory) for synthesizing AVPs (e.g. Type-I or Type-III IFNs) in situ upon interacting with infected target cells, and their activity is proportional to the disease burden. More particularly, upon binding to an infected target cell, the effector cell synthesizes and delivers therapeutic IFNs with spatiotemporal resolution, allowing release of calibrated amounts and only when needed in COVID-19 patients. This controlled release reduces the current adverse events with COVID-19 therapies by providing a living vector as a drug-delivery system for patients.

Type-I and Type-III IFN response, mediated through pattern recognition receptors in host cells, offers the first line of defense against viruses and acts by autocrine/paracrine signaling to induce hundreds of antiviral IFN-stimulated genes (ISGs). The diminished IFN response in case of SARS-CoV-2 has also been implicated for the progression of COVID-19 to different severity levels. While both Type-I and Type-III IFNs generate similar ISGs expression profiles, the timing of their expression during the course of infection differs, and the proportion of their expression to the viral burden determines the severity of the disease. For example, the Type-I IFNs response, which is suppressed by SARS-CoV, is short-lived and occurs during early stages of the infection to serve as a prophylaxis, and Type-III IFNs prevent the progression of disease to severe stages by exerting a long-lasting, non-inflammatory therapeutic response that helps clear systemic infection. Systemically delivered exogenous Type-I IFN has shown therapeutic benefits when infused before the peak viral load. Its use after the infection has however been correlated with the progression of disease to severe stages. Total immunity is additive of Type-I and Type-III IFNs and the challenge is to develop a system that tightly regulates IFN synthesis to autonomously limit their expression with spatiotemporal resolution.

In various embodiments, the genetically engineered effector cell is modular and SARS-CoV-2 antigen-specific. Antigen-specificity can be used to diminish IFN response. Further, the artificial cell-signaling pathway of such genetically engineered effector cells can introduce the capability to serve as vector by producing calibrated amounts of AVPs and inducting intended autocrine and paracrine signaling, upon the genetically engineered effector cell engaging the target antigen. The genetically engineered effector cell can allow for focused synthesis of the biologics at the target site and/or extend treatment duration for better patient outcome by limiting systemic toxicity.

Various embodiments demonstrate the successful implementation of the artificial cell-signaling pathway in a cell line. In some experimental embodiments, the cell line was transformed into a vector for engaging antigen-presenting SARS-CoV-2-infected cells and to trigger the synthesis of calibrated amounts of AVPs in situ, herein sometimes referred to as “effector proteins”.

As used herein, a “genetically engineered effector cell” includes and/or refers to a cell that is genetically engineered or modified to comprise a (i) receptor element, (ii) actuator element, and (iii) effector element, each of which can be modular. As used herein, the terms “modular” and “modularity” include and/or refer to the versatility associated with recombinant sequence domains and the resulting recombinant polypeptides when assembled in various combinations for introduction into an engineered effector cell. The genetically engineered effects cells can be modified for different functionalities by changing portions of the effector element and/or receptor element to develop cell with the different functionalities and for different implementations, such as therapeutic cells, diagnostics cells, and/or reporter cells, as further described herein.

As used herein, “receptor element” includes and/or refers to a polynucleotide sequence encoding a transmembrane receptor, such as a CAR, capable of a specific interaction with a target cell, such as a SARS-CoV-2-infected cell, a genetically engineered target cell that expresses an antigen specific to SARS-CoV-2, a virion or SARs-CoV-2 virus particle or virus-like particle (e.g., natural or genetically engineered), an antibody associated with SARs-CoV-2, and/or other molecules. Depending on the particular application, the receptor element can be reprogrammed by exchanging the single chain variable fragment (scFV) portion of CAR for an extracellular antigen binding domain specific for a different disease-associated antigen or general for an antibody or other molecule (e.g., cytokines, chemokines, proteins). Other receptor elements that can be used include, without limitation, CARs having specificity for antigens associated with autoimmune disorders, CARs having specificity for antigens associated with other viruses or pathogens and/or antibodies.

As used herein, “actuator element” includes and/or refers to a polynucleotide sequence encoding a transcription factor binding site that initiates transcription and translation events downstream of a triggering signal (e.g., binding of the receptor element to a target antigen). In general, the underlying molecular mechanism of the actuator element is based on the intracellular calcium [Ca²⁺]ᵢ dynamics, a mechanism used by almost all types of cells to regulate their functions. Exemplary response elements include, without limitation, NFAT (“nuclear factor of activated T cells”) response element (NFAT-RE), serum response element (SRE), and cyclic AMP response element (CRE).

As used herein, “effector element” includes and/or refers to a polynucleotide sequence encoding an effector protein, and in some instances, an effector protein operably linked to a signal peptide. Example effector proteins include an AVP and a detectable reporter protein. The polynucleotide sequence encoding the effector protein can be, for example, a sequence derived from a human gene, a sequence derived from a gene of a non-human species, a recombinant sequence, a sequence encoding a detectable reporter molecule, a sequence encoding a detectable imaging molecule, a sequence encoding a therapeutic molecule, and the like.

The genetically engineered effector cell into which the receptor element, the actuator element, and the effector element are introduced can be any cell type including human cells or non-human cells (e.g., mammal, reptiles, plants, among others). In this manner, the genetically modified cellular “source” of the modular elements provides a cellular chassis or frame providing, among other things, transcriptional and translational machinery for expression and presentation of the receptor element, the actuator element, and the effector element. In some embodiments, the effector cells can be from a source (e.g., a first human), modified, and administered to an organism that is different than the source (e.g., the host which is a second human). In other embodiments, the cells can be from the source (e.g., a first human), modified, and administered back to the source (e.g., the source is the host).

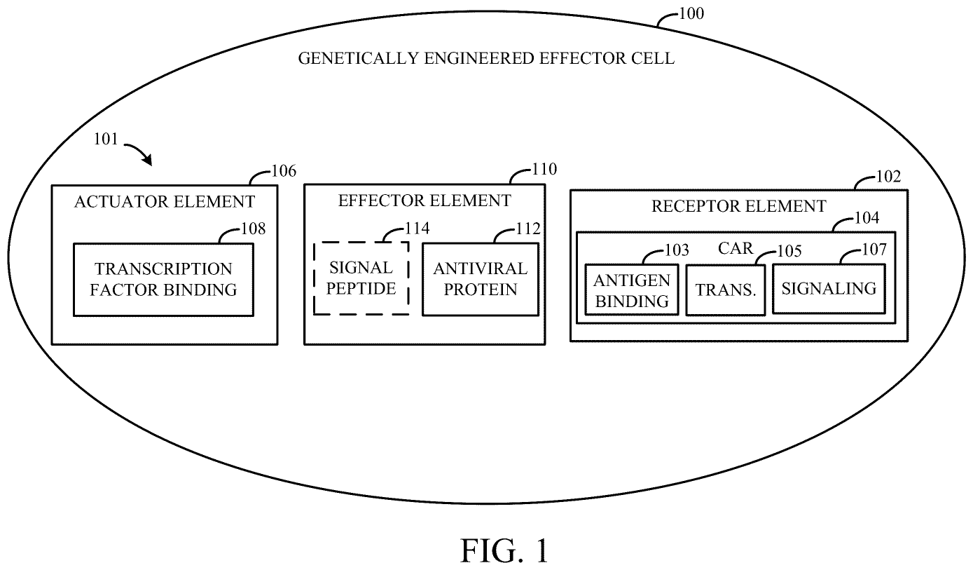

FIG. 1 illustrates an example of a genetically engineered ES effector cell that is specific to SARS-CoV-2, in accordance with the present disclosure. The genetically engineered effector cell 100, herein generally referred to as “an effector cell” can be modular in that genetic elements 102, 106, 110 can be adjusted for different target cells and to synthesize different effector proteins.

The effector cell 100 comprises an exogenous polynucleotide sequence 101 that includes, in operative association, a receptor element 102, an actuator element 106, and an effector element 110. A variety of different types of cells can be genetically modified to form the effector cell 100. Example cells include T-cell, a natural killer cell, a pluripotent stem cell, a multipotent stem cell, an epithelial cell, or a K562 cell. The cell modified to generate the effector cell 100 can include a living cell from an organism, such as a basic membrane-bound unit that contains structural and functional elements. In some embodiments, the exogenous polynucleotide sequence 101 is selected from SEQ ID NOs: 18-22 and 24. In some embodiments, the exogenous polynucleotide sequence 101 includes SEQ ID NO: 18 or SEQ ID NO: 24. However, embodiments are not so limited and the exogenous polynucleotide sequence 101 can include other sequences, such as a sequence with at least 80 percent (%), 85%, 90%, 95%, or 99% sequence identity to one of the sequences set forth in SEQ ID NOs: 18-22 and 24, among others.

The receptor element 102 encodes a CAR 104. A CAR is sometimes called a “chimeric receptor”, a “T-body”, or a “chimeric immune receptor (CAR).” As used herein, a CAR includes and/or refers to an artificially constructed hybrid protein or polypeptide comprising extracellular antigen binding domain(s) 103 of an antibody (e.g., scFv, VHH) operably linked to a transmembrane domain 105 and at least one intracellular signally domain 107. For example, the CAR 104 includes an extracellular antigen binding domain 103 operably linked to the transmembrane domain 105, and the intracellular signaling domain 107. The CAR 104 can be designed to identify an antigen on a surface of a SARS-CoV-2-infected cell, such as a target cell of a host. The CAR 104 can mobilize internal Ca⁺² stores for intracellular Ca⁺² release in response to antigen binding. For example, the extracellular antigen binding domain 103 of the CAR 104 can recognize a surface antigen on a surface of a target cell, such as diseased cells of a host.

In some embodiments, the extracellular antigen binding domain 103 includes SEQ ID NO: 3. In other embodiments, the extracellular antigen binding domain 103 includes any of SEQ ID NOs: 3 and 25-29. However, embodiments are not so limited and the extracellular antigen binding domain 103 can include other sequences, such as a sequence with at least 80%, 85%, 90%, 95%, or 99% sequence identity to one of the sequences set forth in SEQ ID NOs: 3 and 25-29, among other sequences.

As used herein, the extracellular antigen binding domain 103 includes and/or refers to a polynucleotide sequence that is complementary to a target, such as an antigen of the SARS-CoV-2-infected cell. The extracellular antigen binding domain 103 can bind to the surface antigen of the SARS-CoV-2-infected cell, as described above. In some embodiments, the antigen can include the spike glycoprotein (Sgp) or the envelope glycoprotein (Egp) of SARS-CoV-2. In some embodiments, the antigen includes any of SEQ ID NOs: 1-2, or antigens with at least 80%, 85%, 90%, 95%, or 99% sequence identity to one of the sequences set forth in SEQ ID NOs: 1-2, among other sequences.

The transmembrane domain 105 includes and/or refers to a polynucleotide sequence encoding a transmembrane segment of a transmembrane protein, e.g., a type of membrane protein that spans the membrane of a cell, such as the membrane of the cell 100. The transmembrane domain 105 can be derived from a natural polypeptide, or can be artificially designed. A transmembrane domain 105 derived from a natural polypeptide can be obtained from any membrane-binding or transmembrane protein. For example, a transmembrane domain of a T-cell receptor α or β chain, a CD3 chain, CD28, CD38, CD45, CD4, CD5, CD8, CD9, CD16, CD22, CD33, CD37, CD64, CD80, CD86, CD134, CD137, ICOS, CD154, or a GITR can be used.

The intracellular signaling domain 107 includes and/or refers to a polynucleotide sequence encoding any oligopeptide or polypeptide known to function as a domain that transmits a signal to cause activation or inhibition of a biological process in a cell. Example intracellular signaling domains include an intracellular signaling portion of a CD28, an intercellular signaling portion of a 4-1BB, and an intracellular signal portion of a CD3-zeta. In some embodiments, the intracellular signaling domain 107 includes the intracellular signaling portion of CD28, the intercellular signaling portion of 4-1BB, and the intracellular signal portion of CD3-zeta. However, embodiments are not so limited and can include other types and combinations of intracellular signaling domains. For example, the intracellular signaling domain 107 can include encode any molecule that can transmit a signal into a cell when the extracellular antigen binding domain 103 present within the same molecule binds to (interacts with) the antigen.

Generally, the antigen binding domain 103 of a CAR 104 has specificity for a particular antigen expressed on the surface of a target cell of interest, a SARS-CoV-2-infected cell. As described above, the extracellular binding domain 103 is capable of binding to an antigen includes any oligopeptide or polypeptide that can bind to the antigen, and includes, for example, an antigen-binding domain of an antibody and a ligand-binding domain of a receptor. The extracellular antigen binding domain 103 binds to and interacts with the antigen present on a cell surface of the SARS-CoV-2-infected cell, and thereby imparts specificity to an effector cell 100 expressing the CAR 104. In some embodiments, the receptor element 102 encodes a CAR 104 comprising an extracellular antigen binding domain 103 having specificity for the Sgp or Egp associated with SARS-CoV-2.

The actuator element 106 encodes a transcription factor binding site 108. The transcription factor binding site 108 includes and/or refers to binding site for a protein that upregulates synthesis of the AVP 112 in response to the extracellular antigen binding domain 103 of the CAR 104 binding to the antigen of the SARS-CoV-2-infected cell. The transcription factor binding site 108 can bind to transcription factors as triggered by [Ca²⁺], which as described above, are caused to release in response to the antigen binding. In some embodiments, the transcription factor binding site 108 is selected from a nuclear factor of activated T-cell (NFAT) response element, a serum response element (SRE), and a cyclic AMP response element (CRE).

The actuator element 106 can thereby include a sequence for binding the factors triggered by [Ca²⁺], and can trigger amplified synthesis of the AVP 112 (and/or another type of protein) in response to [Ca²⁺]ᵢ rise.

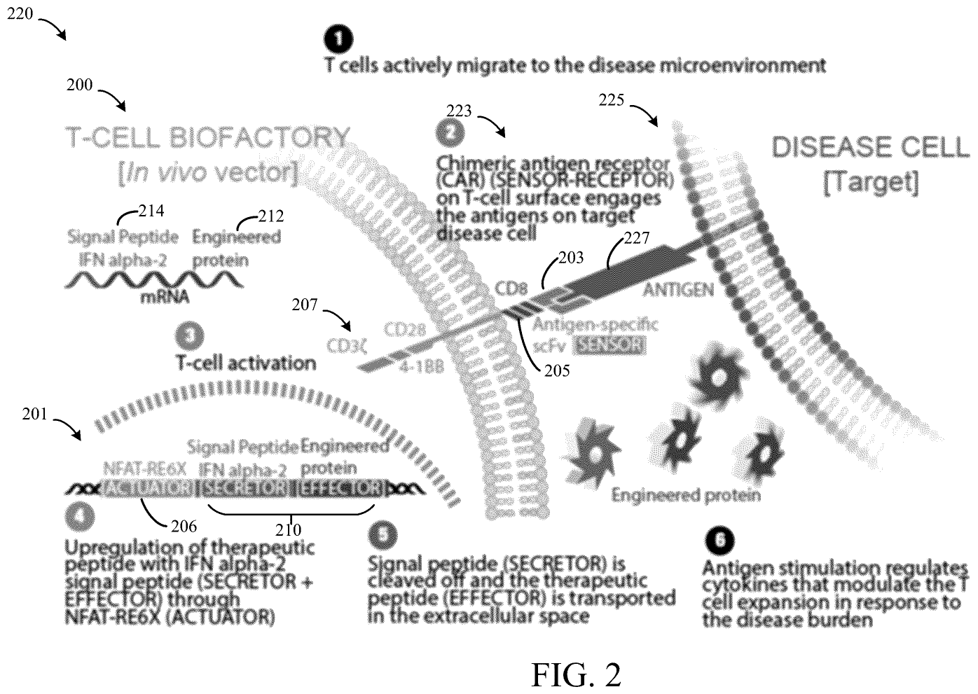

In some embodiments, the actuator element 106 encodes a NFAT transcription factor binding site for a transcription factor protein. In some embodiments, the actuator element 106 encodes a set of NFAT transcription factor binding sites, such as at least two transcription factor binding sites, three transcription factor binding sites, or six transcription factor binding sites (e.g., six NFAT-RE, among other amounts. NFAT transcription factor family consists of five members NFATc1, NFATc2, NFATc3, NFATc4, and NFAT5. See Sharma S et al. (2011) PNAS, 108(28); Hogan P G et al. (2010) Ann Rev Immunol, 28; Rao A, Hogan P G (2009) Immunol Rev, 231(1); Rao A (2009) Nat Immunol, 10(1), M. R. Muller and A. Rao, Nature Reviews Immunology, 2010, 10, 645-656; M. Oh-Hora and A. Rao, Curr. Opin. Immunol., 2008, 20, 250-258. Crabtree & Olson E N (April 2002), Cell 109 Suppl (2): S67-79, which are each hereby incorporated herein in their entireties for their teaching. NFATc1 through NFATc4 are regulated by calcium signaling. Calcium signaling is critical to NFAT activation because calmodulin, a well-known calcium sensor protein, activates the serine/threonine phosphatase calcineurin. The underlying molecular mechanism of this strategy is based on intracellular Ca⁺² ([Ca²⁺]ᵢ) dynamics (as further shown by FIG. 2). The [Ca²⁺]ᵢ dynamics are common to almost all cell types, and the approach is thus broadly applicable. The [Ca²⁺]ᵢ rise from CAR-mediated stimulation of cells leads to dephosphorylation of the nuclear factor of an activated effector cell 100 (through Ca⁺²/calmodulin-dependent serine phosphatase calcineurin), which then translocated to the nucleus and interact with the NFAT-RE to upregulate expression of the AVP 112. In parallel, the NFAT-RE also performs its natural function of inducing Interleukin-2 in the activated effector cell 100 that regulates clonal expansion proportional to the disease burden.

The effector element 110 encodes the AVP 112, and in some instances, encodes the AVP 112 operably linked to a signal peptide 114. As further illustrated herein, in some embodiments, the signal peptide 114 is upstream of the AVP 112 (or other protein). The signal peptide 114 can be non-native to the AVP 112. For example, the AVP 112 can be unable to secrete into the extracellular environment without the addition of the signal peptide 114. However, embodiments are not so limited and in some embodiments, the AVP 112 includes a native signal peptide.

For example, the AVP 112 can (natively) include the signal peptide 114. In other embodiments, the native signal peptide of the effector protein 112 can be removed and a non-native signal peptide 114 can be added. In some embodiments, the AVP 112 can be encoded by and/or include SEQ ID NO: 4, SEQ ID NO: 5, SEQ ID NO: 6 or SEQ ID NO: 7. As such, in some embodiments, the effector element 110 can include one or more of SEQ ID NO: 4, SEQ ID NO: 5, SEQ ID NO: 6, and SEQ ID NO: 7.

However, embodiments are not so limited and the effector element 110 can include other sequences, such as a sequence with at least 80%, 85%, 90%, 95%, or 99% sequence identity to one or more of the sequences set forth in SEQ ID NOs: 4-7, among other sequences.

As used herein, the terms “secretor”, “secretory peptide and “signal peptide” are used interchangeable and include and/or refer to a peptide that assists or directs the synthesized AVP 112 into the extracellular environment (e.g., assists with translocating the effector element 110). The signal peptide 114 can be operably linked or fused to the AVP 112 for release into the extracellular environment. In this manner, the signal peptide 114 can direct movement of the AVP 112 outside of the effector cell 100. A signal peptide 114 is particularly advantageous when included in the effector cell 100 expressing an AVP 112 that is unable to and/or minimally able to translocate natively, where the AVP 112 can remain inside the effector cell 100 in the absence of the signal peptide 114 and/or can translocate at a rate below a threshold. Generally, signal peptides are located at the N-terminus of nascent secreted proteins and characteristically have three domains: (1) a basic domain at the N-terminus, (2) a central hydrophobic core, and (3) a carboxy-terminal cleavage region. Any signal peptide can be used. For example, the signal peptide 114 can be the signal peptide of Interleukin-6 or Interleukin-2.

In various embodiments, in response to the extracellular antigen binding domain 103 of the CAR 104 binding to the antigen of the target cell (e.g., a target host cell), the effector cell 100 is configured to activate, and to synthesize and secrete the AVP 112. For example, the effector cell 100 can synthesize and secrete an amount of the AVP 112 as a function of an amount of the SARS-CoV-2-infected cell and/or an amount of other antigen-presenting cells in the environment (e.g., the extracellular environment), such as secreting an amount of the AVP 112 in the environment that is proportional to the number of SARS-CoV-2-infected cells present in the environment (e.g., in a sample or in situ).

The AVP 112 can include a variety of different types of proteins, which can be used to provide treatment for SARS-CoV-2 or to prevent infection. An AVP protein includes and/or refers to a protein that provides a therapeutic effect to a host from a viral infection. Example antiviral proteins include IFN, such as a Type-I IFN and/or Type-III IFN, Molnupiravir, Ivermectin, Nitazoxanide, Hydroxychloroquine, Chloroquine, and Azithromycin, among others. In some embodiments, the AVP 112 can stimulate production of other therapeutic proteins in the host. For example, the activation of the effector cell 100 can regulate stimulation of cytokines and/or other proteins in the host.

Different parts of the genetic elements 102, 106, 110 of the effector cell 100 can be modular and other parts can be conserved (e.g., may not change for different implementations). For example, in some embodiments, the intracellular signaling domain 107, the actuator element 106, and the optional signal peptide 114 are constant domains, and the extracellular antigen binding domain 103 and the AVP 112 are variable domains. As an example, the extracellular antigen binding domain 103 can be changed for different targets and/or the AVP 112 can be changed to cause in situ synthesis of different proteins, while the intracellular signaling domain 107, the actuator element 106, and the signal peptide 114 remain the same for the different implementations. Keeping parts conserved can reduce production time. However, embodiments are not so limited, and any part of the effector cell 100 can be modified.

In some embodiments, the effector cell 100 can include multiple (e.g., two or more) of some or all of the genetic elements 102, 106, 110. For example, the effector cell 100 can include multiple receptor elements 102, multiple actuator elements 106, and/or multiple effector elements 110. In some embodiments, multiplicity takes the form of providing multiple genetically engineered effector cells (e.g., a plurality of cells) modified as described herein to a host to provide more than one therapeutic task for treating or preventing SARS-CoV-2 and/or for other purposes.

In some embodiments, the effector element 110 can encode multiple effector proteins, such as at least two AVPs, an AVP and a detectable reporter protein, or at least two detectable reporter proteins, among other types of effector protein combinations. For example, two effector proteins can be encoded linked together by a 2A linker peptide. A 2A linker peptide, as used herein, includes and/or refers to a peptide which induces ribosomal skipping during translation of a protein complex (e.g., encoding of two proteins or peptides linked by the 2A linker peptide) in a cell, such that the protein complex is translated into two proteins that independently fold. Example 2A linker peptides include F2A, P2A, E2A, and T2A, among others. Such peptides are generally 18-22 amino acids long, and derived from viruses.

In some embodiments, the actuator element 106 is connected to and/or associated with the effector element 110. In some embodiments, the exogenous polynucleotide sequence 101 includes the actuator element 106 connected to the effector element 110 connected to the receptor element 102, which are all formed on a single plasmid vector. For example, the exogenous polynucleotide sequence 101 can include the actuator element 106 connected to and upstream from the effector element 110, and the effector element 110 connected to and upstream or downstream from the receptor element 102, wherein the signal peptide 114 is upstream from the AVP 112. In other embodiments, the receptor element 102 can be on a different plasmid vector than the actuator element 106 and the effector element 110.

FIG. 2 illustrates an example of a genetically engineered effector cell and a sequence of events triggered when in a diseased environment, in accordance with the present disclosure. The genetically engineered effector cell 200, herein generally referred to as the “effector cell 200” for ease of reference, can be used as or act as a living vector to synthesize the AVP 212 using the artificial cell-signaling pathway and/or to trigger a sequence of events 220. The effector cell 200 synthesizes the engineered AVP 212 in situ upon interacting with the antigen-presenting target cell 225, as shown at 222.

As previously described, the effector cell 200 can comprise a polynucleotide sequence 201 including the receptor element encoding the extracellular antigen binding domain 203, transmembrane domain 205, and the intracellular signaling domain 207, the actuator element 206 encoding the transcription factor binding site (e.g., NFAT), and the effector element 210 encoding the AVP 212 and, optionally, the signal peptide 214. The polynucleotide sequence 201 can comprise a single plasmid (e.g., a single construct including each of) comprising three constant domains (e.g., the actuator element 206, the signal peptide 214, and portions of the receptor element, such as the transmembrane domain 205 and the intracellular signaling domain 207), and two variable domains (e.g., the antigen binding domain 203 (labeled as the “sensor”) and AVP 212) arranged in cis. In other embodiments, the polynucleotide sequence 201 can comprise multiple plasmids such as a first plasmid comprising the actuator element 206 and the effector element 210, and a second plasmid comprising the receptor element.

The constant domains can be configured to provide functionality to the effector cell 200. The constant domains form part of the intracellular signaling pathway and include a transmembrane molecule (e.g., transmembrane domain 205) that mobilizes the calcium-dependent transcriptional machinery (e.g., actuator element 206) to upregulate the effector transgene (e.g., AVP 212) fused to the signal peptide 214 that assists in transporting the effector transgene into the extracellular space 223. In various embodiments, the AVP 212 can include a native signal peptide, which forms part of the AVP 212.

The variable domains can be responsible for the applicability of the effector cell 200 to different diseases, target cells, therapy, and/or other applications. For example, the variable domains can impart specificity to the effector cell 200 against particular diseases. The variable domains can include a variable heavy-light (V_{H}-V_{L}) chain (e.g., the antigen binding domain 203, labeled as the “sensor”) to identify the antigen biomarker on the target cell (e.g., labeled “diseased cell”) independent of the peptide-major histocompatibility complex, and the effector transgene (e.g., AVP 212). The variable domains are modular. For example, the antigen binding domain 203 can be exchanged or revised to reprogram the effector cell 200 to target biomarkers specific to different cell-based diseases. As another example, the AVP 212 can be exchanged or revised with different therapeutic transgenes, such as for neutralizing the pathology that activated the effector cell 200 and essentially creating an off-shelf living vector, which is enhanced further by the innate cytolytic activity of effector cells.

In some embodiments, the receptor element encodes a CAR. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives effector cells expressing CARs the ability to recognize antigen independent of antigen processing. Referring to FIG. 2, expression of a transmembrane CAR enables an effector cell 200 to sense and bind to the target antigen 227 expressed on the surface of target cell 225, such as SARS-CoV-2 infected cells. Binding of the CAR and target surface antigen 227 on the target cell 225 activates the effector cell 200, which triggers an activation cascade leading to the expression of the AVP 212. For example, expression of the AVP 212, or other effector protein is autonomously expressed as part of the effector cell 200 activation cascade in response to binding of the transmembrane receptor to the antigen 227 presented on the target cell 225.

More particularly, the effector cell 200 expressing a CAR can bind to a SARS-CoV-2 specific antigen via the CAR, and in response a signal is transmitted into the effector cell 200, and as a result, the effector cell 200 is activated. The activation of the effector cell 200 expressing the CAR is varied depending on the kind of target cell and an intracellular domain of the CAR, and can be confirmed based on, for example, release of a cytokine, improvement of a cell proliferation rate, change in a cell surface molecule, or the like as an index. For example, release of a cytotoxic cytokine (e.g., tumor necrosis factor, a lymphotoxin, etc.) from the activated effector cell 200 causes destruction of a target cell 225 expressing an antigen 227. In addition, release of a cytokine or change in a cell surface molecule stimulates other immune cells, for example, a B cell, a dendritic cell, a natural killer cell, and a macrophage.

As shown by FIG. 2, an example sequence of events 220 triggered by or related to the effector cell 200 includes (1) the effector cell 200 actively migrating to the diseased environment, (2) the CAR on the effector cell 200 surface engaging the antigen 227 of the target cell 225 that comprises a SARS-CoV-2-infected cell, (3) the effector cell activation, (4) upregulation of the AVP 212 with the signal peptide 214 through the NFAT, (5) signal peptide 214 is cleaved off and AVP 212 is transported into the extracellular space 223, and (6) antigen stimulation regulates cytokines that modulate cell expansion in response to the disease burden.

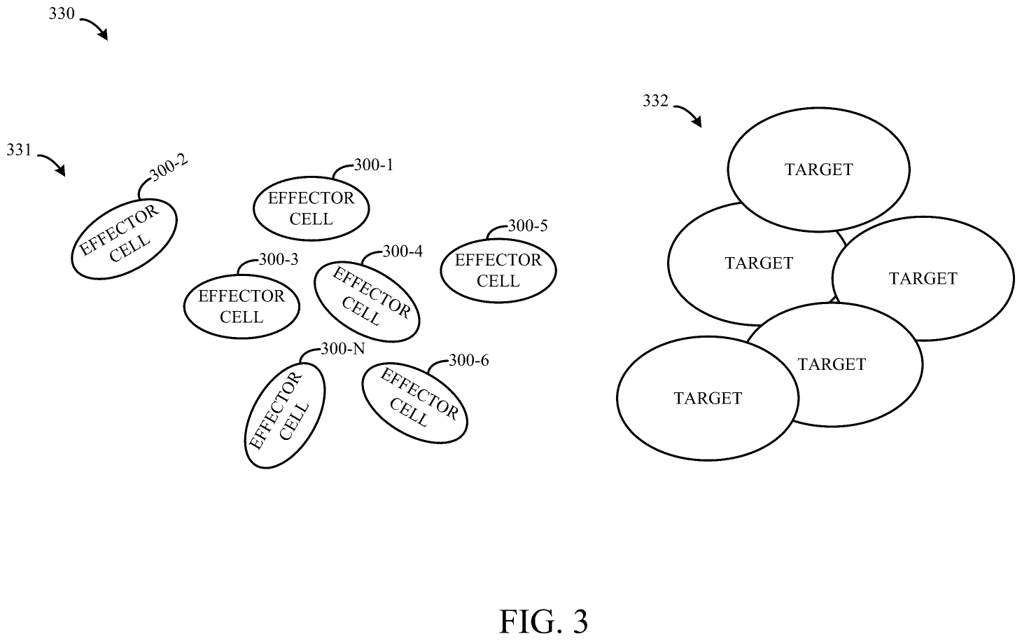

FIG. 3 illustrates an example of a population of genetically engineered effector cells in a diseased environment, in accordance with the present disclosure. The population 331 can include a plurality of genetically engineered effector cells 300-1, 300-2, 300-3, 300-4, 300-5, 300-6, 300-N (herein generally referend to as “the effector cells 300” for ease of references). Each of the effector cells 300 can include at least substantially the same components and features as the effector cell 100 of FIG. 1, the details of which are not repeated for ease of reference.

In the example illustrated by FIG. 3, the environment is an extracellular space 330 that includes (a presence of) target cell(s) 332, such that the space 330 can be referred to as a diseased environment. The population 331 of effector cells 300 can bind to the antigens of the target cell(s) 332 via the antigen binding domain of the CAR. In response to the binding, the effector cells 300 can activate and, in response, synthesize and secrete a calibrated amount of the effector protein based on a presence of the target cell(s) 332. For example, the calibrated amount of the effector protein is a function of an amount of the target cell 332 present in a plurality of (host) cells, such as in an extracellular space 330 or in a sample. As previously described, the calibrated amount of the effector protein can be proportional to the amount of the target cell 332. Although the extracellular space 330 illustrates effector cells 300 and the target cells 332, the extracellular space 330 and the plurality of (host) cells can further include other normal and/or diseased cells, among other non-cellular components.

In some embodiments, the target cell(s) 332 include SARS-CoV-2-infected cells and the effector protein includes an AVP. In such embodiments, the antigen can include the Sgp or the Egp of SARS-CoV-2 and the AVP may cause action on the SARs-CoV-2 to treat or prevent an infection. More particularly, the AVP may co-opt other surrounding cells to produced IFN stimulated genes (ISGs), many of which are antiviral. The effector cells 300 may generate a calibrated amount of AVP, where the calibrated amount of the AVP is a function of an amount of the SARS-COV-2-infected cell (or other antigen-presenting cells) present in a plurality of cells or in a sample.

In some embodiments, different effector cells of the population 331 can encode different AVPs and/or can encode for multiple AVPs or other effector proteins. For example, a first subset of the population 331 of effector cells 300 can include the effector element that encodes a first AVP and a second subset of the population 331 of effector cells 300 can include the effector element that encodes the second AVP. In other embodiments, each of the effector cells 300 or a sub-portion thereof can include effector elements that encode the first AVP bound to the second AVP by a 2A linker peptide. In some embodiments, the first and second AVPs include Type-I IFN and Type-III IFN, however, embodiments are not so limited.

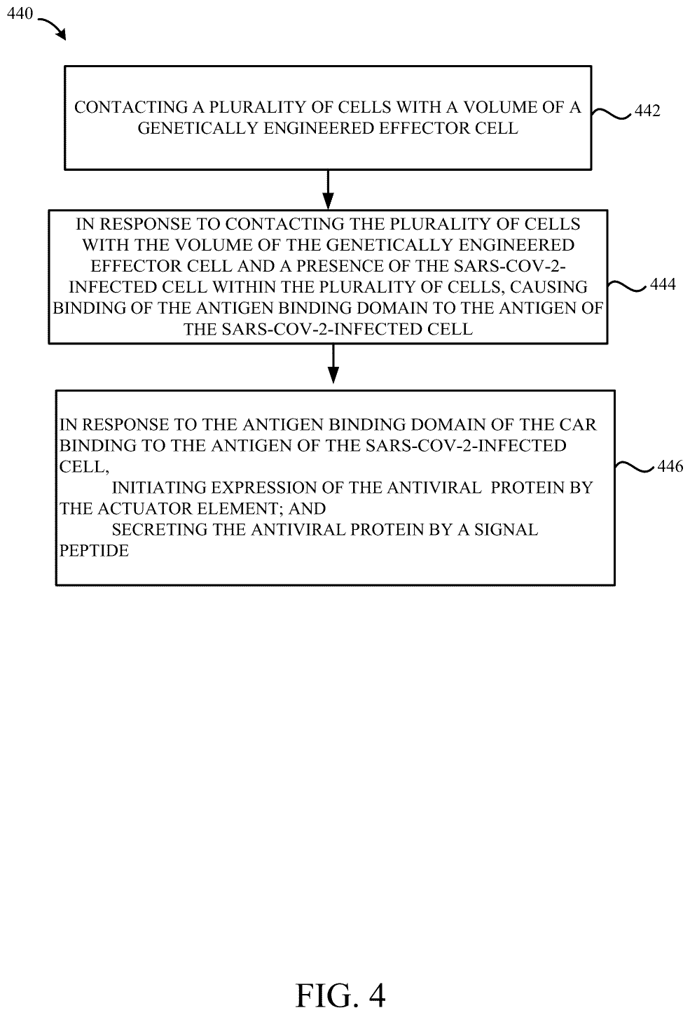

FIG. 4 illustrates an example method of contacting a plurality of cells with a volume of a genetically engineered effector cell, in accordance with the present disclosure. The method 440 can be implemented using the effector cell 100 illustrated by FIG. 1 and/or the population 331 of effector cells 300 illustrated by FIG. 3.

At 442, the method 440 includes contacting a plurality of cells with a volume of a genetically engineered effector cell. The cells can be contacted by contacting a sample with or administering the volume of the genetically engineered effector cell to a host, such as a patient. The genetically engineered effector cell can include at least some of substantially the same components and features as previously described by the effector cell 100 of FIG. 1, the details of which are not repeated for ease of reference.

At 444, in response to contacting the plurality of cells with the genetically engineered effector cell and a presence of the SARS-CoV-2-infected cell within the plurality of cells, the method 440 includes causing binding of the antigen binding domain to an antigen on a surface of the SARS-CoV-2-infected cell. The plurality of cells, including the infected cell, can include cells of a host (e.g., host cells and target host cells).

At 446, in response to the antigen binding domain (of the CAR) binding to the antigen of the SARS-CoV-2-infected cell, the method 440 includes initiating expression of (e.g., transcription and translation of) the AVP by the actuator element to synthesize the AVP, and secreting the AVP by a signal peptide. The signal peptide can be native to the AVP or can be non-native and is encoded by the effector element. In some embodiments, the method 440 can further include, in response to the antigen binding domain of the CAR binding to the antigen of the SARS-CoV-2-infected cell, activating the effector cell and, in response, synthesizing and secreting a calibrated amount of the AVP based on the presence of the SARS-CoV-2-infected cell. As previously described, the calibrated amount of the AVP can be a function of (e.g., is proportional to) an amount of the SARS-CoV-2-infected cell present within the plurality of cells in the environment.

In some embodiments, the method 440 further includes detecting expression of the AVP. Detectable expression of the AVP can indicate the presence of the target cell. In some embodiments, as described above, the AVP can be bound to a detectable reporter protein by a 2A linker peptide.

The AVP can act directly on the target cell, such as killing the target cell, or indirectly by co-opting other therapeutic proteins or cells in the body. As previously described, the AVP can include an IFN and/or a set of AVPs or other effector proteins.

Various experiments, as further described below, were directed to developing a cell-based therapeutic that induces the desired IFN response found to be a critical contributor to severe COVID-19. Example results can include assessing the production of two different IFNs (Type-I IFN-β1a and Type-III IFN-λ2) from two effector cell, determining the protection offered to the SARS-CoV-2 infected host cells, and demonstrating the upregulation of antiviral ISGs in response to these IFNs. The results indicate that the effector cell has specificity toward SARS-CoV-2 and SARS-CoV-1, and it can also be activated by the infectious SARS-CoV-2 virion particles to produce a desired effector protein. This effector cell technology can be applied beyond SARS-CoV-2. The engineered effector can be genetically reprogrammed to target other pathogenic viruses and regulate the production of desired therapeutic peptides. The effector cell utilizes the synthetic pathway that bypasses the natural pathway of using pattern recognition receptors to trigger IFN signaling and is often compromised in the pathogenic viral infections. The approach represents a substantive departure from the status quo and offers and effective means to keep future pandemics in check.

Like other pathogenic viruses, SARS-CoV-2 virus evades the innate immune response of a host, which is critical in the establishment of an early antiviral defense. It has been reported that SARS-CoV-2 virus causes a delayed IFN response thereby contributing to the severity of COVID-19. As such, recombinant IFN proteins, with broad antiviral activity, have been applied in the management of COVID-19 and other viral infections. Several studies have demonstrated that recombinant IFNs can reduce viral replication in vitro and improve clinical outcomes in animal studies. Clinical trials for both Type-I and Type-III IFNs have also been undertaken in COVID-19 patients to show their safety and efficacy. However, due to the increased hyperinflammation caused by infusion of exogeneous IFNs, most of these clinical studies underlines dose, timing, and disease stage as critical for a better clinical outcome. The effector cell is designed to mitigate these concerns and make the IFNs available with spatiotemporal resolution. The effector cell activates only when it engages with infected cells and synthesizes IFNs proportional to the infection burden. This active regulation of therapeutics with localized precision can reduce the inefficacies due to the suboptimal levels of IFNs or adverse events caused by its excess, both of which have been reported for systemic infusions.

In example embodiments, exogenous IFN treatments can be beneficial for COVID-19 patients. Experiments show that delivery of IFNs through the effector cell is protective at the cellular levels when administered at the right time and with right amounts. The effector cell presents a unique route of administering calibrated amounts of IFNs with localized precision and therefore extends the possibility of avoiding undesired side effects in COVID-19 patients. This cell-based intervention circumvents the ex vivo production of clinical-grade IFNs and its platform nature further adds to its impact and can be redirected to target other pathogens and immunological diseases, e.g., cancers, autoimmune disorders. Integration of next generation safety and kill switches to ensure efficient and quick removal of the infused T-cells is crucial for the safe translation and application in clinics.

IFNs clear most viruses and once approved through the regulatory agencies, the treatment can be deployed quickly in case of an outbreak. The deployment of a pre-approved intervention thus offers to boost public's confidence in health policies. While systemic infusion of IFNs has shown clinical benefit against many viruses, unregulated delivery causes inflammation, tissue damage, and multiorgan failure. Effector cells can address the challenge of regulating the bioavailability of antiviral IFNs by engineering a T-cell line for synthesizing calibrated amounts of the desired IFNs with spatiotemporal resolution and making it safe for use by rendering it non-proliferative. The IFN-producing effector cell offers to be an antiviral intervention that can be deployed against many emerging pathogens by making a change of exchanging the binding portion of the receptor element to redirect specificity against the envelop protein or other antigens of the new pathogens.

Various embodiments are directed to a pharmaceutical composition comprising a genetically engineered effector cell and a pharmaceutically acceptable carrier or excipient, such as the effector cell 100 of FIG. 1 and/or the population 331 of effector cells 300 of FIG. 3.

For example, an effector cell composition, such as a pharmaceutical composition, can comprises a plurality of the genetically engineered effector cells described herein and an acceptable carrier, diluents, or excipient (e.g., a pharmaceutically acceptable carrier, diluent, excipient or a combination thereof). The means of making such a composition have been described in the art (see, for instance, Remington's Pharmaceutical Sciences, 16th Ed., Mack, ed. (1980)). Preferably, the composition is prepared to facilitate the administration of the cells into a living organism. In some embodiments, the pharmaceutical composition comprises a plurality of genetically engineered effector cells as described herein and, for example, a balanced salt solution, preferably Hanks' balanced salt solution, or normal saline.

Examples are not limited to effector cells which are used for therapeutics. In some examples, the effector cell can include an effector element that encodes a detectable reporter protein, which can be used for diagnostics. For example, the effector cell can be used to diagnose a current infection, and can be referred to as a diagnostic cell. In some embodiments, the effector cell can be used to test for an antibody response, such as testing for past infections or immunity, and can be referred to as a reporter cell.

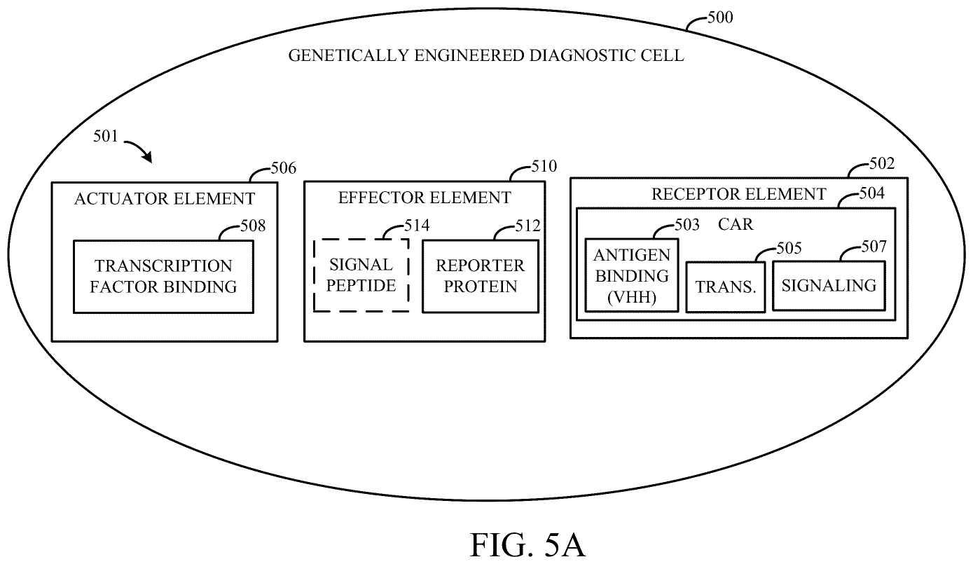

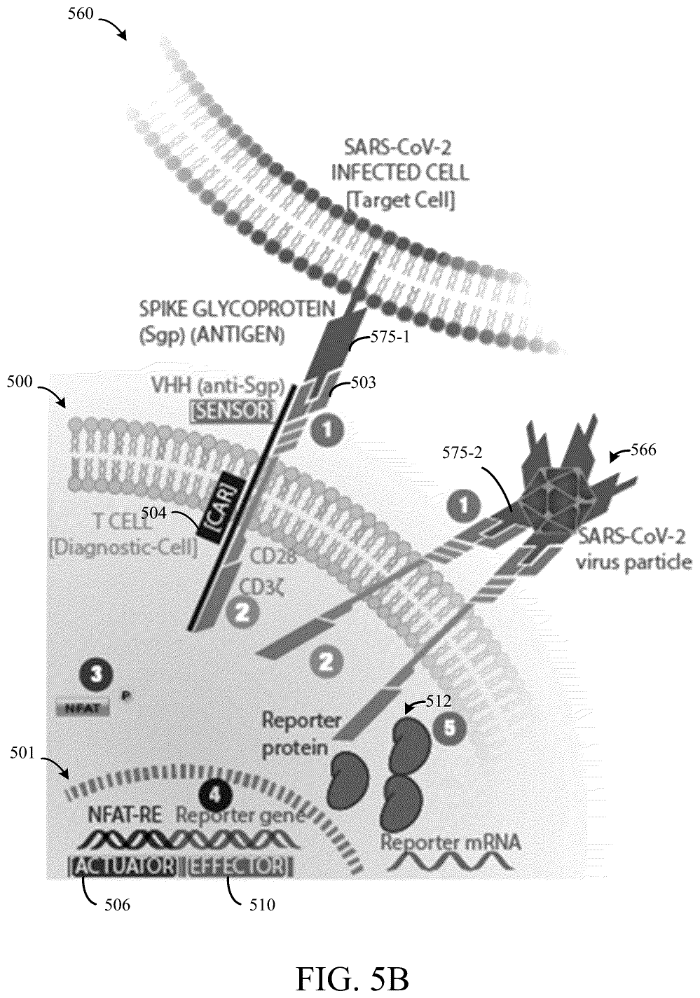

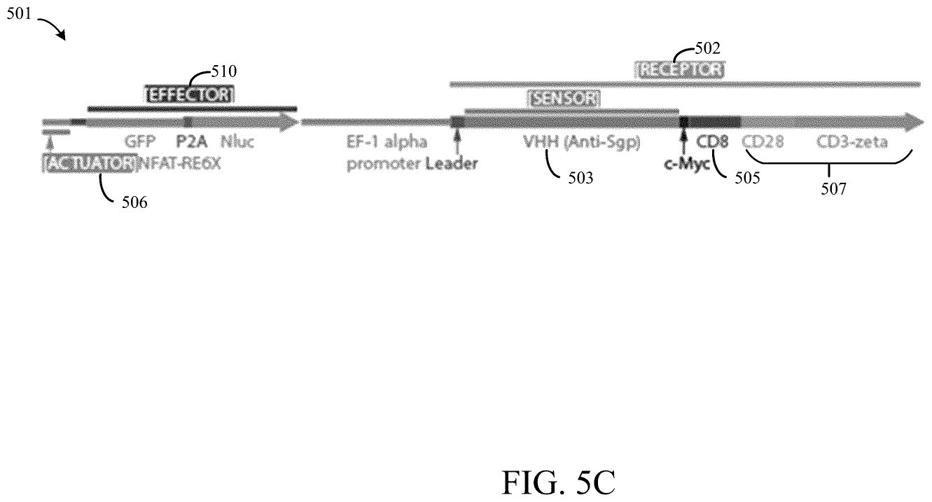

FIGS. 5A-5C illustrate an example of a genetically engineered diagnostic cell and a sequence of events triggered for providing an antigen test, in accordance with the present disclosure. For ease of reference, the genetically engineered diagnostic cell 500 is generally referred as “the diagnostic cell 500.”

In various embodiments, the diagnostic cell 500 can be used to perform an antigen test to diagnose an infection for SARS-CoV-2. Asymptomatic viral transmission may be responsible for a viral spread that escapes clinical surveillance strategies, posing an obstacle to controlling outbreaks. Aggressive triaging, rapid contact tracing, and testing/retesting of hosts suspected of having the infection are the cornerstones of successful pandemic mitigation. The diagnostic cell 500 can use virus-specific molecular signatures to diagnose hosts with active infections and can be used in both clinically symptomatic and asymptomatic stages. While the various embodiments describe use for SARS-CoV-2, the diagnostic cell 500 can be modified to be specific for other virus.

As shown by FIGS. 5A-5C, the diagnostic cell 500 can include at least some of substantially the same components and features as the effector cell 100 of FIG. 1, with the receptor element encoding a different extracellular antigen binding domain and the effector element encoding different proteins. For example, the diagnostic cell 500 comprises an exogenous polynucleotide sequence 501 that includes, in operative association, a receptor element 502, an actuator element 506, and an effector element 510. A variety of different types of cells can be genetically modified to form the diagnostic cell 500 (e.g., T-cells, NK cells, etc.). In some embodiments, the exogenous polynucleotide sequence 501 is selected from any of SEQ ID NOs: 18, 40-44, 46, 48, 50, 52, and 54. However, embodiments are not so limited and the exogenous polynucleotide sequence 501 can include other sequences, such as a sequence with at least 80%, 85%, 90%, 95%, or 99% sequence identity to one of the sequences set forth in SEQ ID NOs: 18, 40-44, 46, 48, 50, 52, and 54, among others.

The receptor element 502 encodes a CAR 504. The CAR can include an extracellular binding that includes a single-domain heavy chain (VHH) region of an antibody specific to an antigen of SARS-CoV-2, such as the Sgp. However, embodiments are not so limited, and in some embodiments, a variable-heavy light (VH-VL) portion of the scFv of antibody specific to a SARS-CoV-2 antigen can be used. The extracellular antigen binding domain 503 can recognize Sgp 575-1, 575-2, or other antigen(s), on a surface of a target cell, such as a SARS-CoV-2 infected cell 560 or a SARS-CoV-2 virus particle 566. In some embodiments, the extracellular antigen binding domain 503 is selected from SEQ ID NOs: 3 and 25-29 (e.g., for SARs-Cov-1 and/or SARs-Cov-2), SEQ ID NO: 31 (e.g., for Ebola), SEQ ID NO: 33 (e.g., for Marburg), SEQ ID NO: 35 (e.g., for Chikungunya), SEQ ID NO: 37 (e.g., for Nipah), and SEQ ID NO: 39 (e.g., for West Nile), among combinations thereof. In some embodiments, the extracellular antigen binding domain 503 is selected from SEQ ID NOs: 3 and 25-29. However, embodiments are not so limited and the extracellular antigen binding domain 503 can include other sequences, such as a sequence with at least 80%, 85%, 90%, 95%, or 99% sequence identity to one or more of the sequences set forth in SEQ ID NOs: 3 and 25-29, among others.

The extracellular antigen binding domain 503 can bind to an antigen, such as any of SEQ ID NOs: 1-2 (e.g., for SARs-Cov-1 and/or SARs-Cov-2), SEQ ID NO: 30 (e.g., for Ebola), SEQ ID NO: 32 (e.g., for Marburg), SEQ ID NO: 34 (e.g., for Chikungunya), SEQ ID NO: 36 (e.g., for Nipah), and SEQ ID NO: 38 (e.g., for West Nile), among combinations thereof. However, embodiments are not so limited and can include a variety of different antigens.

As previously described, the extracellular antigen binding domain 503 is operably linked to the transmembrane domain 505 and the intracellular signaling domain 507. The transmembrane domain 505 and the intracellular signaling domain 507 can include at least some of substantially the same components and features as the transmembrane domain 105 and the intracellular signaling domain 107 of FIG. 1, the common features and attributes not being repeated for ease of reference.

The actuator element 506 can include at least some of substantially the same components and features as the actuator element 106 of FIG. 1, the common features and attributes not being repeated for ease of reference. For example, the actuator element 506 can encode a transcription factor binding site 508, such as a NFAT transcription factor binding site for a transcription factor protein and/or a set of NFAT-REs.

The effector element 510 of the diagnostic cell 500 encodes a detectable reporter protein 512. For example, and as further illustrated by FIG. 5C, the effector element 510 can encode two detectable reporter proteins which are linked by a 2A linker peptide. In some embodiments, the detectable reporter protein(s) can have a native signal peptide. In other embodiments, the non-native signal peptide 514 can be added to the effector element 510, as previously described, or may otherwise not be required. In some embodiments, the detectable reporter protein 512 can be encoded by and/or include any of SEQ ID NOs: 8-14, and combinations thereof. As such, in some embodiments, the effector element 510 can include one or more of SEQ ID NO: 8, SEQ ID NO: 9, SEQ ID NO: 10, SEQ ID NO: 11, SEQ ID NO: 12, SEQ ID NO: 13, and SEQ ID NO: 14. However, embodiments are not so limited and the effector element 510 can include other sequences, such as a sequence with at least 80%, 85%, 90%, 95%, or 99% sequence identity to one or more of the sequences set forth in SEQ ID NOs: 8-14, among others.

As used herein, a detectable reporter protein includes and/or refers to a protein that is detectable upon expression, such as a protein that provides an optical, electrical, and/or other type of detectable signal. Example detectable reporter peptides include luciferase or a bioluminescent variant thereof, Green Fluorescent Protein (GFP) or a fluorescent variant thereof, and lacZ or a colorimetric variant thereof.

In various embodiments, and as shown by FIG. 5B, in response to the extracellular antigen binding domain 503 of the CAR 504 binding to the antigen 575-1, 575-2 of the target cell (e.g., a SARS-CoV-2 infected cell 560 or a SARS-CoV-2 virus particle 566), the diagnostic cell 500 is configured to activate, and to synthesize and secrete the detectable reporter protein 512. For example, the diagnostic cell 500 can synthesize and secrete an amount of the detectable reporter protein 512 as a function of an amount of the SARS-CoV-2-infected cell 560 and/or SARS-CoV-2 virus particle 566 in the environment (e.g., the extracellular environment), such as in a sample or in situ. In some embodiments, a nasal or saliva sample can be taken from a host (e.g., a subject) and used to detect for infection.

The detectable reporter protein 512 is detectable and can be used to diagnose a host for a currently occurring SARS-CoV-2 infection, such as for performing an antigen test. Additionally, the amount (e.g., intensity) of the detectable reporter protein 512 can be used to assess the disease burden, as the amount is proportional to the amount of antigens present in the environment.

Although the above described use for SARS-CoV-2, embodiments are not so limited and can include additional antigen tests performed as a panel.

FIG. 5C illustrates an example polynucleotide sequence 501 of the diagnostic cell of FIG. 5A. As shown, the polynucleotide sequence 501 encodes an actuator element 506 connected to and upstream from an effector element 510 that encodes at least one detectable reporter protein (e.g., GFP linked to Nluc by a P2A linker peptide). The polynucleotide sequence 501 further includes a receptor element 502 that encodes an antigen binding domain 503 (e.g., the VHH that is specific to Sgp) operably linked to the transmembrane domain 505 and the intracellular signaling domain 507. In various embodiments, the receptor element 502 may be encoded on a separate plasmid vector from the actuator element 506 and the effector element 510.

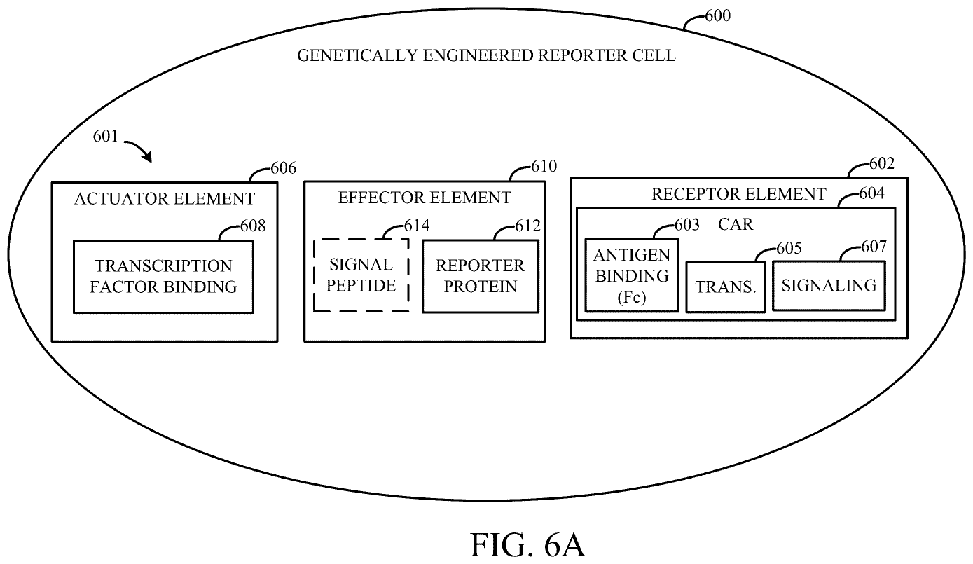

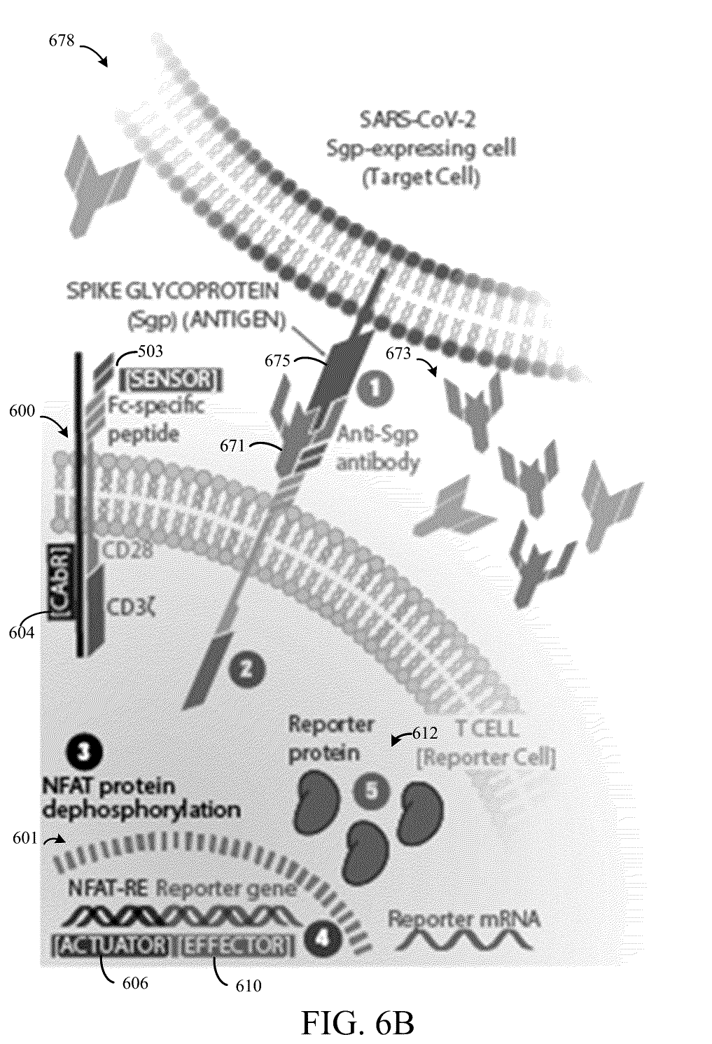

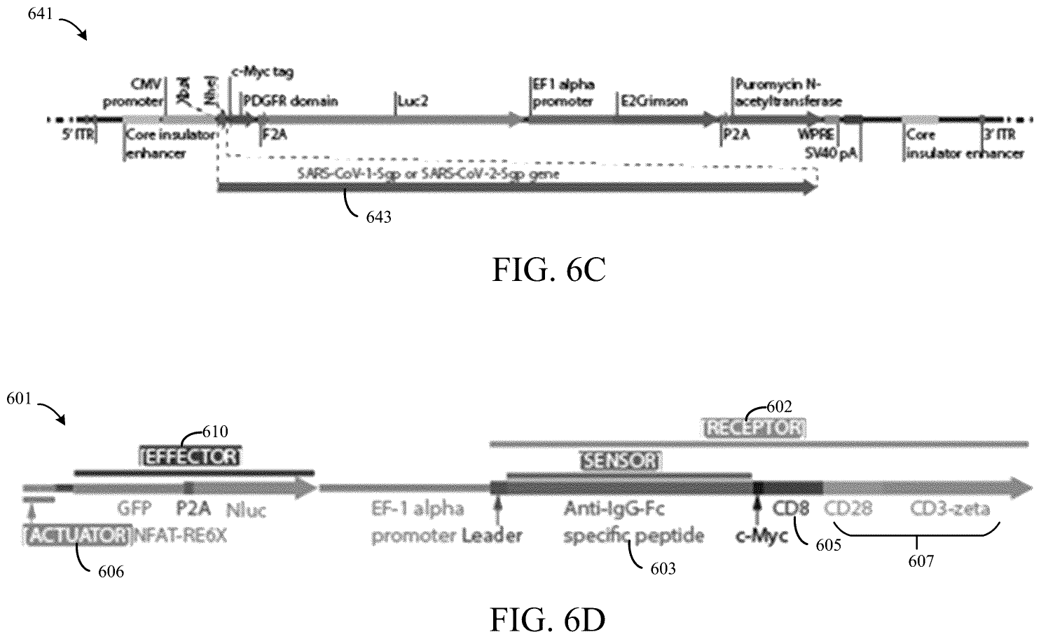

FIGS. 6A-6D illustrate an example of a genetically engineered reporter cell and a sequence of events triggered for providing an serology test, in accordance with the present disclosure. For ease of reference, the genetically engineered reporter cell 600 is generally referred as “the reporter cell 600.”

As shown by FIGS. 6A-6D, the reporter cell 600 can include at least some of substantially the same components and features as the effector cell 100 of FIG. 1, with the receptor element encoding a different extracellular binding domain and the effector element encoding different proteins. The effector cell 600 comprises an exogenous polynucleotide sequence 601 that includes, in operative association, a receptor element 602, an actuator element 606, and an effector element 610. A variety of different types of cells can be genetically modified to form the effector cell 600. In some embodiments, the exogenous polynucleotide sequence 601 can include SEQ ID NO: 56, however examples are not so limited. However, embodiments are not so limited and the exogenous polynucleotide sequence 601 can include other sequences, such as a sequence with at least 80%, 85%, 90%, 95%, or 99% sequence identity to SEQ ID NO: 56, among other sequences.

As shown by FIGS. 6B-6D, two different cells types can be generated. The first includes the reporter cell 600, which is a type of effector cell. A target cell 678 can additionally be generated, which can be a cell that is modified to express or present a target antigen 675 and is herein referred to as a “genetically engineered target cell” or an “antigen-presenting target cell”. In some embodiments, the target cells 678 can be referred to as pseudo-infected cells, in that the cells are modified to present or express the antigen 675 but are not infectious. In some embodiments, genetically engineered antigen-presenting virus-like particles or virions are generated, which are modified to present or express the antigen 675 but are not infectious and can be referred to as pseudo-virus-like particles. However, embodiments are not limited to detecting antibodies associated with a virus, and in some embodiments, the target cells 678 can include a target antigen 675 which is specific to other types of molecules, such as cytokines, chemokines, and/or proteins. In such embodiments, the target antigen 675 on the target cells 678 (as well as the antigen binding domain of the reporter cell 600) can include peptides or scFvs that are specific to a segment (e.g., epitope) on the molecule to be detected. The cells can be modified to form target cells 678 using an exogenous polynucleotide sequence 641 that encodes an antigen 643.

In some embodiments, the target cells 678 can include antigens 678 such as any of SEQ ID NOs: 1-2 (e.g., for SARs-Cov-1 and/or SARs-Cov-2), SEQ ID NO: 30 (e.g., for Ebola), SEQ ID NO: 32 (e.g., for Marburg), SEQ ID NO: 34 (e.g., for Chikungunya), SEQ ID NO: 36 (e.g., for Nipah), and SEQ ID NO: 38 (e.g., for West Nile), among combinations thereof. In some embodiments, the target cells 678 can be encoded and/or formed using any of SEQ ID NOs: 16-17 and 23 (e.g., for SARs-Cov-1 and/or SARs-Cov-2), SEQ ID NO: 45 (e.g., for Ebola), SEQ ID NO: 47 (e.g., for Marburg), SEQ ID NO: 49 (e.g., for Chikungunya), SEQ ID NO: 51 (e.g., for Nipah), and SEQ ID NO: 53 (e.g., for West Nile), among others. However, embodiments are not so limited and the target cells 678 and/or antigens 678 of the target cells 678 can include other sequences, such as a sequence with at least 80%, 85%, 90%, 95%, or 99% sequence identity to one or more of the sequences set forth in SEQ ID NOs: 1-2, 16-17, 23, 30, 32, 34, 36, 38, 45, 47, 49, 51, and 53, among other sequences.

The receptor element 602 encodes a CAR 604. The CAR can include an extracellular antigen binding domain 603 that includes an anti-IgG-Fc specific peptide. The extracellular antigen binding domain 603 can recognize any IgG antibody 673, and is not specific to a target antibody 671. As such, the reporter cell 600 can be used to test for antibodies specific to different pathogens by generating different target cells 678 which present the target antigen 675. While other antibodies 673 can bind to the binding domain 603, the environment can be controlled to only include target cells 678 expressing the target antigen 675, and not include other antigens or targets. As such, the reporter cell 600 is only activated in response to the presence of IgG antibodies specific to the target antigen 675, e.g., Sgp. As described above, embodiments are not limited to testing or detecting antibodies and/or antibodies specific to pathogens. In some embodiments, the extracellular antigen binding domain 603 can include a peptide or ScFv that recognizes and/or is specific to a segment (e.g., epitope) on another type of molecule to be detected, such as cytokines, chemokines, proteins, among other molecules. In such embodiments, the extracellular antigen binding domain 603 and the antigen of the target cells 678 can be specific to two different and unique segments, e.g., epitopes, of the molecule to be detected. In some embodiments, the extracellular antigen binding domain 603 can include SEQ ID NO: 55. However, embodiments are not so limited and the extracellular antigen binding domain 603 can include other sequences, such as a sequence with at least 80%, 85%, 90%, 95%, or 99% sequence identity to SEQ ID NO: 55, among other sequences.

As previously described, the extracellular antigen binding domain 603 operably linked to the transmembrane domain 605 and the intracellular signaling domain 607. The transmembrane domain 605 and the intracellular signaling domain 607 can include at least some of substantially the same components and features as the transmembrane domain 105 and the intracellular signaling domain 107 of FIG. 1, the common components and features not being repeated for ease of reference. The actuator element 606 can include at least some of substantially the same components and features as the actuator element 106 of FIG. 1, the common components and features not being repeated for ease of reference. For example, the actuator element 606 can encode a transcription factor binding site 608, such as a NFAT transcription factor binding site for a transcription factor protein and/or a set of NFAT-REs.

The effector element 610 of the reporter cell 600 encodes a detectable reporter protein. For example, and as further illustrated by FIG. 6D, the effector element 610 can encode two detectable reporter proteins which are linked by a 2A linker peptide, as previously described by FIG. 5B. In some examples, the effector element can optionally encode a signal peptide 614. In some embodiments, the detectable reporter protein 600 can be encoded by and/or include any of SEQ ID NOs: 8-14, and combinations thereof. As such, in some embodiments, the effector element 610 can include one or more of SEQ ID NO: 8, SEQ ID NO: 9, SEQ ID NO: 10, SEQ ID NO: 11, SEQ ID NO: 12, SEQ ID NO: 13, and SEQ ID NO: 14. However, embodiments are not so limited and the effector element 610 can include other sequences, such as a sequence with at least 80%, 85%, 90%, 95%, or 99% sequence identity to one or more of the sequences set forth in SEQ ID NOs: 8-14, among other sequences.

In various embodiments, as shown by FIG. 6B, in response to the extracellular antigen binding domain 603 of the CAR 604 binding to the target antibody 671, e.g., the anti-Sgp antibody, and the target antibody 671 binding to the antigen-presenting target cell 678, e.g., SARS-CoV-2 Sgp-expressing cell, the reporter cell 600 is configured to activate, and to synthesize and secrete the detectable reporter protein 612. For example, the reporter cell 600 can synthesize and secrete an amount of the detectable reporter protein 612 as a function of an amount of the target antibody 671 present in the environment, such as in a sample or in situ.

The detectable reporter protein 612 is detectable and used to assess for immune response to the pathogen or other purposes, such as for performing a serology test. For example, blood (among other types of samples, such as nasal samples) can be taken from a host and used to perform the test. Additionally, the amount (e.g., intensity) of the detectable reporter protein 612 can be used to assess immune response, as the amount can be proportional to the amount of antibody present in the environment.

Although the above described use for SARS-CoV-2, embodiments are not so limited and can include additional antigen and/or serology tests performed as a panel. In other embodiments, the target molecule detected can include a molecule other than an antibody, as described above.

FIG. 6C illustrates an example polynucleotide sequence 641 used to generate an antigen-expressing modified cell as shown by FIG. 6A. As shown, the polynucleotide sequence 641 encodes the antigen 643, such as the SARS-CoV-1 Sgp or the SARS-CoV-2 Sgp.

FIG. 6D illustrates an example polynucleotide sequence 601 of the reporter cell of FIG. 6A. As shown, the polynucleotide sequence 601 encodes an actuator element 606 connected and upstream from an effector element 610 that encodes at least one detectable reporter protein (e.g., GFP linked to Nluc by a P2A linker peptide). The polynucleotide sequence 601 further includes a receptor element 602 that encodes a binding domain 603 (e.g., the Anti-IgG-Fc specific peptide) operably linked to the transmembrane domain 605 and the intracellular signaling domain 607. In various embodiments, the receptor element 602 may be encoded on a separate plasmid vector from the actuator element 606 and the effector element 610.

Some embodiments are directed to methods of forming the genetically engineered effector cells, such as genetically engineering or modifying an effector cell to include the components and features as described by the effector cell 100 of FIG. 1, diagnostic cell 500 of FIG. 5A, and/or reporter cell 600 of FIG. 6A.

The genetically engineered effector cells and cell compositions provided herein have properties advantageous for use in a variety of in vitro, ex vivo, and in vivo applications. For example, in vitro uses of the effector cells and cell compositions provided herein include, without limitation, detecting target cells on the basis of antigens expressed on the surface of the target cells. The target cell can be a cell infected by a pathogen such as a virus or bacterium, a cell type associated with an immune response to a pathogen (e.g., antibody). Also, the target (host) cell can be a cell type associated with any other pathology for which the affected (host) cell having aberrant expression of a cell surface antigen relative to an unaffected (host) cell. Methods for using the genetically engineered effector cells or cell compositions for in vitro target cell detection are described above and further below. In various embodiments, multiple effector cells that are targeted to different pathogens or different immune responses to the different pathogens can be used to form panel antigen test or panel serology tests for the different pathogens. For example, a single antigen test can be used to test for SARS-CoV-2, the flu, and various common cold strains.

Ex vivo uses of the genetically engineered effector cells and cell compositions provided herein include, without limitation, early disease detection and companion diagnostic or therapeutic applications for the disease target cells identified on the basis of antigens expressed on the surface of the disease target cells. For example, the cells can be used for ex vivo applications in companion diagnostics for cancer immunotherapy. By way of example, the effector cell engineered with NFAT_RE6X with Nluc-2A-GFP can be engineered to express different types of CARs. The expression of Nluc when CAR engages its target antigen versus the non-specific Nluc expression can inform on the comparative and quantitative robustness of each CAR for its efficiency to cause the intended on-target effect versus unintended off-target effects. Methods for using the genetically engineered effector cells or cell compositions in ex vivo therapeutic applications are described further below.

In vivo applications of the genetically engineered effector cells and cell compositions provided herein include, without limitation, in vivo methods for localized therapy at a disease site (e.g., targeted therapy for ovarian cancer) or site of pathogen infection (e.g., targeted therapy for cells infected by SARs-Cov-2, dengue virus, Zika virus, West Nile virus, yellow fever, HIV, or a hepatitis virus (e.g., HepB, HepC)).

Various embodiments are directed to a panel of different types of genetically engineered effector cells, such as a plurality of effector cells engineered with different effector proteins and/or extracellular antigen binding domains (among other differences), and which are used to simultaneously target different cells and/or secrete different effector proteins.

In some embodiments, a method of detecting a target cell comprises (a) contacting a genetically engineered effector cell to a cell population, and (b) detecting expression of the effector protein, wherein detectable expression of the effector protein indicates the presence of the target cell of interest. In some embodiments, the effector cell includes a NFAT-RE and a reporter protein, and in the presence of the target cell in the contacted cell population, the genetically engineered effector cell binds to a surface molecular antigen on the target cell and activates the NFAT-RE; and (b) detecting expression of the reporter protein, wherein detectable expression of the reporter protein indicates the presence of the target cell.

In embodiments, the detected target cell is a virus-infected host cell such as, for example, a SARS-CoV-2-infected cell. In some such embodiments, the surface molecular antigen expressed on the virus-infected cell can be a SARS-CoV-2 virus-specific Egp or Sgp. For example, the antigen-recognizing portion of the CAR is modified or exchanged to quantitatively assess different viral pathogens such as SARS-CoV-2, dengue virus (DENV), West Nile (WNV), and Yellow Fever (YFV). In some embodiments, the methods harness the translational machinery of the infected host cell to process viral RNA into a virus-specific antigen that is detectable by the genetically engineered effector cell.

Some embodiments are directed to methods of treating or preventing a disease using genetically engineered effector cells expressing a CAR as a therapeutic agent. For example, provided herein are methods comprising administering a genetically engineered effector cell expressing the CAR as an active therapeutic agent. The disease against which the effector cell expressing the CAR is administered is not particularly limited as long as the disease shows sensitivity to the effector cell. In some embodiments, a genetically engineered effector cell expressing the CAR binds to an antigen expressed on the surface of a target cell that targeted to be decreased or eliminated for treatment of the aforementioned diseases, that is, a viral antigen for effector, is administered to treat or prevent such diseases. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, methods described herein can provide any amount of any level of treatment or prevention of SARS-CoV-2 in a mammal. Furthermore, the treatment or prevention provided by example methods can include treatment or prevention of one or more conditions or symptoms of the virus, e.g., SARS-CoV-2, being treated or prevented. Also, for purposes herein, “prevention” can encompass delaying the onset of the disease, or a symptom or condition thereof.

In some embodiments, genetically engineered effector cells are administered to a host (e.g., subject) in need thereof as a composition comprising the genetically engineered effector cells and a suitable carrier, diluent, or excipient as described herein. Any appropriate method of providing modified CAR-expressing cells to a host can be used for methods described herein. In some embodiments, methods for providing effector cells to a host can be adapted from clinical protocols for cellular and adoptive immunotherapy for infusion of donor-derived immune cells into a human host. In some embodiments, an adapted clinical protocol suitable for methods provided herein comprises obtaining effector cells from a host, genetically engineering (e.g., modifying) effector cells to express a CAR and NFAT-RE regulated AVP transgene as described herein, and infusing the genetically engineered effector cells back into the host. A host, as used herein, includes and/or refers to any organism, such as a human, an animal (e.g., mammal, reptile, bird), insect, plant, among others, and which can be a subject of a study or test and/or a patient. A “subject” is sometimes interchangeably used with “host”. Host cells include cells obtained from the host.

Administration of the genetically engineered effector cells provided herein can be administered by any appropriate route, including, without limitation, administration intravenously, intratumorally, intramuscularly, subcutaneously, intraperitoneally, intra-arterially, or into an afferent lymph vessel, by parenteral administration, for example, by injection or infusion. In some embodiments, where genetically engineered effector cells or populations of such effector cells are administered, the effector cells can be cells that are allogeneic or autologous to the host, such as a mammal. Preferably, the effector cells are autologous to the host.

In some embodiments, the genetically engineered effector cells comprise a CAR that detects an antigen on a pathogen-infected cell (e.g., SARS-CoV-2) or an antibody triggered in response to prior infection, and a NFAT response element to induce expression of a reporter polypeptide. Such embodiments can be used for transfusion medicine to detect the presence of emerging pathogens and/or to identify and track natural immunity.

As used herein, a target cell (sometimes herein interchangeably referred to as a “target cell of a host”, “target cell of interest”, “a diseased cell”, or “a target disease cell”) includes and/or refers to a cell of interest associated with a living organism (e.g., a biological component of interest) or a modified live cell in a test environment (e.g., genetically modified test cells or other antigen-present cells in solution). An antigen of the target cell includes and/or refers to a structure (e.g., binding site) of the target cell which the antigen binding domain of the receptor element can bind to (e.g., has an affinity for). The effector cell can be from a variety of different type of cells, such as human and non-human cells, and sometimes herein referred to as “the source”. As used herein, the terms “genetically modified” and “genetically engineered” are used interchangeably and include and/or refer to a prokaryotic or eukaryotic cell that includes an exogenous polynucleotide, regardless of the method used for insertion. In some embodiments, the effector cell is modified to comprise a non-naturally occurring nucleic acid molecule that is created or modified by the hand of man (e.g., using recombinant deoxyribonucleic acid (DNA) technology) or is derived from such a molecule (e.g., by transcription, translation, etc.). An effector cell that contains an exogenous, recombinant, synthetic, and/or otherwise modified polynucleotide is considered to be a genetically engineered effector cell.

“Nucleic acid”, as used herein, includes and/or refers to a “polynucleotide,” “oligonucleotide,” and “nucleic acid molecule,” and generally means a polymer of DNA or ribonucleic acid (RNA), which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid can encode additional amino acid sequences that do not affect the function of the CAR and polynucleotide and which may or may not be translated upon expression of the nucleic acid by a host cell.

Nucleic acids can be obtained using any suitable method, including those described by Maniatis et al., Molecular Cloning: A Laboratory Manual, Cold Spring Harbor, N.Y., pp. 280-281 (1982) and/or U.S. Patent Application Publication No. US2002/0190663, each of which are herein fully incorporated in their entireties for their teachings. Nucleic acids obtained from biological samples typically are fragmented to produce suitable fragments for analysis.

Nucleic acids and/or other moieties can be isolated. As used herein, “isolated” includes and/or refers to separate from at least some of the components with which it is usually associated whether it is derived from a naturally occurring source or made synthetically, in whole or in part. Nucleic acids and/or other moieties of the invention can be purified. As used herein, “purified” includes and/or refers s separate from the majority of other compounds or entities. A compound or moiety can be partially purified or substantially purified. Purity can be denoted by a weight by weight measure and can be determined using a variety of analytical techniques such as but not limited to mass spectrometry, HPLC, etc.

### Experimental Embodiments

A number of experimental embodiments were conducted to generate genetically engineered effector cells including therapeutic cells, diagnostic cells, and reporter cells, and to characterize the effector cell functionality. Example constructs used to generate genetically engineered cells include the nucleotide sequences set forth in SEQ ID NOs: 1-56. SEQ ID NOs: 1-56 are each synthetic DNA.

In various embodiments, immune cells are modified to form effector cells that, upon engaging the antigen-presenting target cells (e.g., natural or genetically modified), produce non-endogenous proteins for exerting a target effect locally. In some embodiments, two different types of T-cells are modified to form effector cells that synthesize anti-viral Type-1 (IFN-α2b, IFN-β1a) and Type-III (IFN-λ2; IFN-λ1), which are sometimes herein referred to as “T-cell Biofactories” or “cell biofactory”. Prophylactic and therapeutic effects of the IFN produced from the T-cell Biofactory were determined on SARS-CoV-2 infected cells. The ISGs activated in the host cells were investigate, when exposed to the Type-I or Type-III IFN producing T-cell Biofactory. The radiation dose (γ-radiation) used to render the T-cell Biofactory non-proliferative was identified, while conserving the cell-based IFN production, to mitigate potential oncogenesis risks. This ensures safe administration of the T-cell Biofactory that can have translational implications and can be made available in compliance with cGMP for Phase I/II clinical trials.

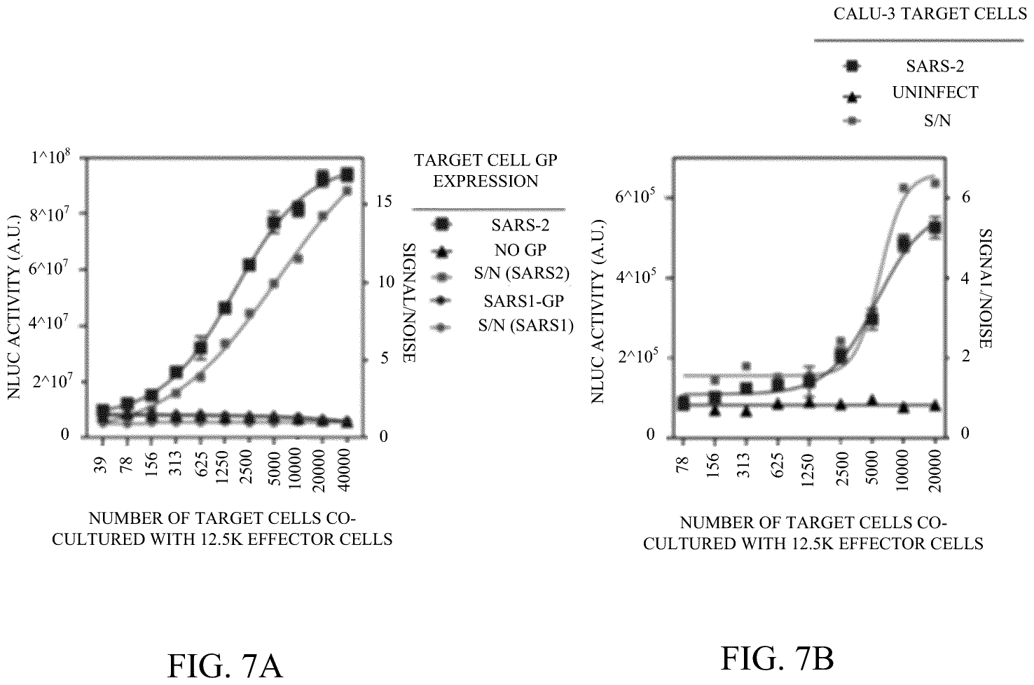

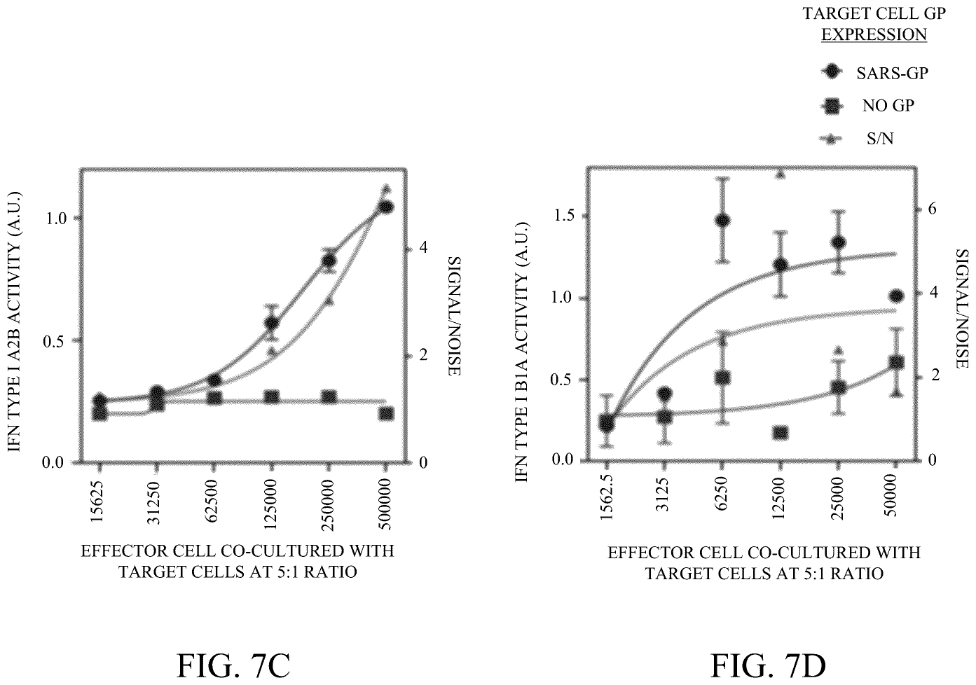

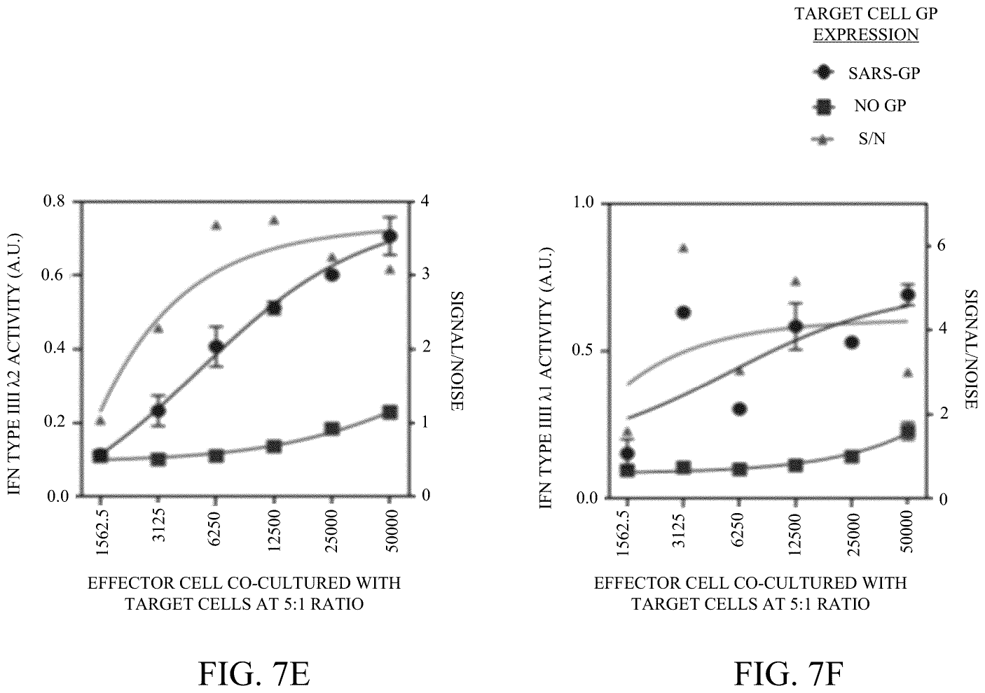

FIGS. 7A-7F illustrate plots characterizing genetically engineered effector cells function with specificity against SARS-CoV-2 antigens, in accordance with the present disclosure.

An effector cell was engineered to target viral infection to develop cell-based delivery of AVO therapeutics. A fast-growing cell line, Jurkat E6-1, was modified to include a receptor element, an actuator element, and an effector element. The receptor elements encoded a CAR with specificity to the Sgp of SARS-CoV-2, sometime referred to as “Sgp-CAR”. The Sgp-CAR triggers the downstream pathway, e.g., activates the actuator element encoding a NFAT-RE to synthesize an AVP of IFN encoded by the effector element. Different effector cells, sometimes referred to as cell biofactories, were developed that encoded Type-I IFN and Type-III IFN. Target cells were artificially prepared to express the Sgp from SARS-CoV-1 and SARS-CoV-2, which can be referred to as genetically modified target cells. The genetically modified target cells were co-cultured with the Spg-specific effector cell, which resulted in production of Type-I IFN or Type-III IFN upon engaging with the genetically modified target cells. IFN-sensitive reporter cells (Type-I, Type-III) were used to assess the output of the Sgp-specific effector cell in the context of efficacy and off-target activity. Validations were further conducting using target cells with infectious SARS-COV-2 virus particles in BSL3.

FIG. 7A illustrates the results of co-culturing anti-SARS-CoV-2 effector cells with HEK cell lines that model viral infection through expression of viral-specific Sgp and FIG. 7B illustrates the results of results of co-culturing anti-SARS-CoV-2 effector cells with SARS-CoV-2 infected Calu-3 cells, activates nLuciferase reporter expression, as shown by the plot of FIG. 7B. Activation of the effector cell is mediated through the Sgp that is specific for the virus and is proportional to mock or live viral load. Further engineering of the virus-specific effector cell replaced nLuciferase reporter with Type-I IFN (as shown by FIGS. 7C and 7D) and Type-III IFN (as shown by FIGS. 7E and 7F). Coculture of IFN-effector cell with modeled or artificial SARS-CoV-2-infected target cells induces interferon secretion proportional to the number of effector cells.

The modified effector cell can serve as a therapeutic to treat severe cases and a prophylactic to prevent the progression of mild/moderate cases to severe cases of SARS-CoV-2 and related diseases. Such effector cell can be used as a readily-available stockpile of broad-spectrum antiviral cells that, in event of an epidemic of bioweapon attach, can be rapidly deployed and activated using extracorporeal devices without the need for developing vaccines specific for each viral pathogen.

As may be appreciated, embodiments were not limited to SARS-CoV-2.

Different effector cells, diagnostic cells, and reporter cells were generated to assess different viruses and other pathogens.

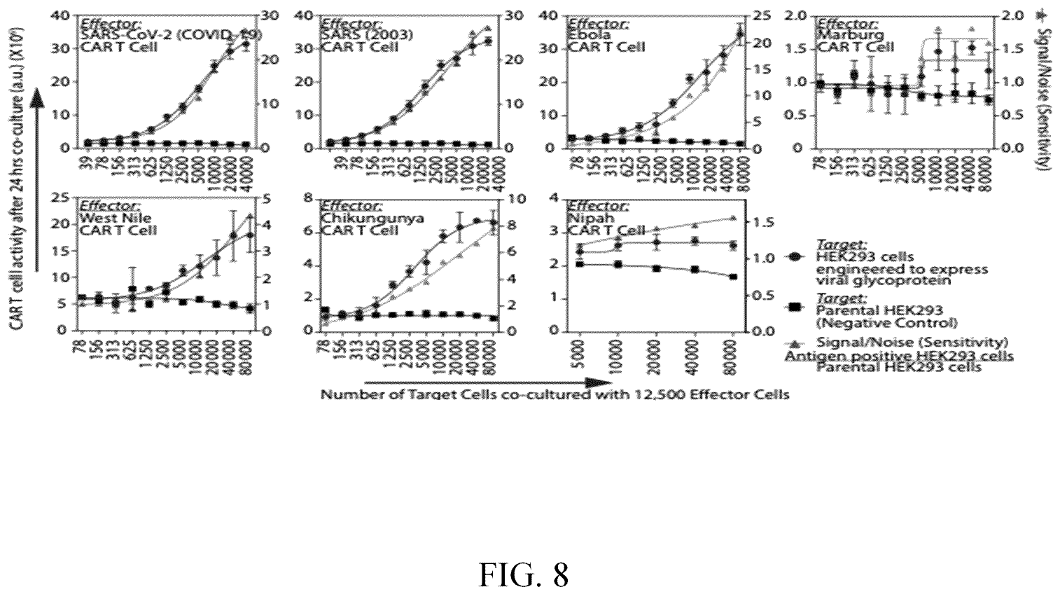

FIG. 8 illustrates plots characterizing genetically engineered effector cell function with specificity against different pathogens, in accordance with the present disclosure. For example, effector cells which produce IFNs were generated that were specific to different pathogens including SARS-CoV-2, SARS-CoV-1, Ebola, Marburg, West Nile, Chikungunya, and Nipah.

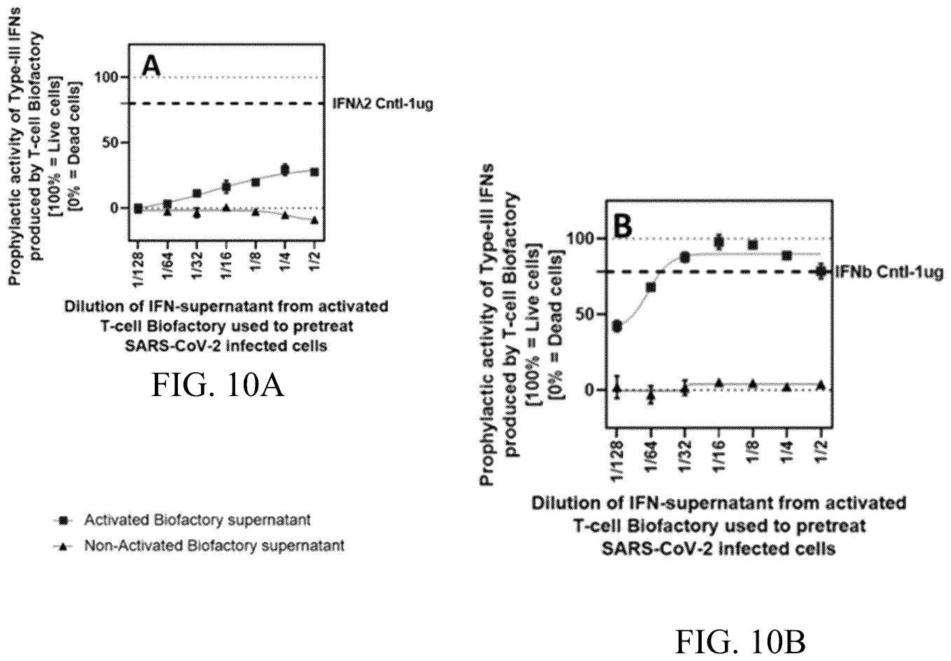

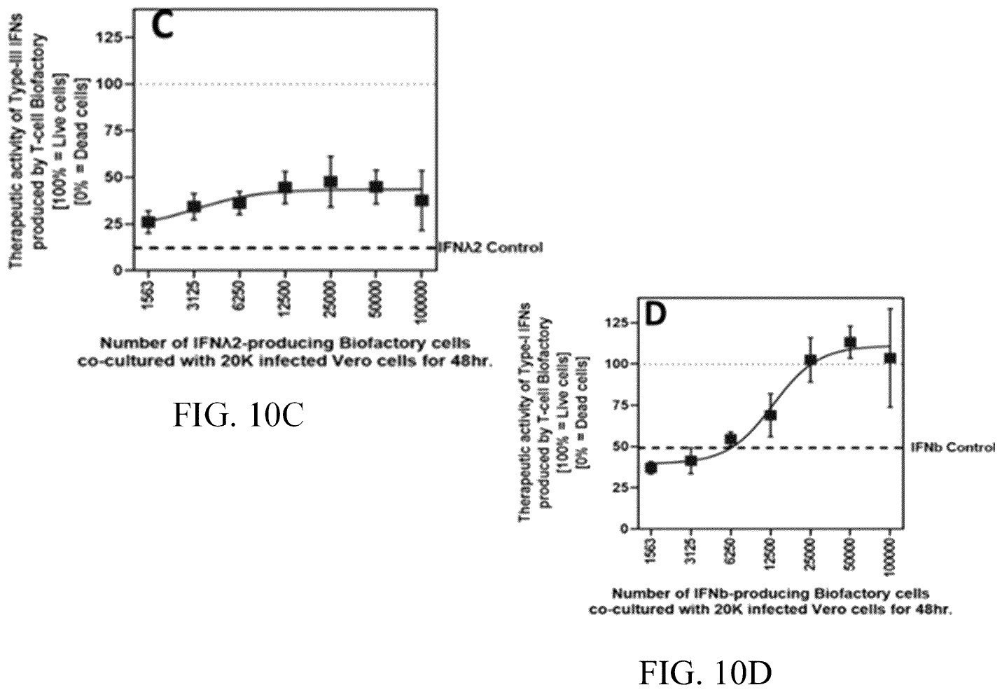

Various embodiments were directed to assessing the artificial cell-signaling pathway, which comprises three constant domains (portions of the receptor element (e.g., transmembrane domain, and intracellular signaling domain), actuator element, signal peptide) and two variable domains (the CAR of the receptor element, effector element) domains arranged in cis, was used to generate the effector cell from T-cells that produces Type-I or Type-III IFNs upon detecting the Sgp as an antigenic biomarker on the surface of host cells infected with SARS-associated coronaviruses, which can be referred to as target cells. The constant domains provide functionality to the effector cell and include a transmembrane molecule that mobilizes the T-cell activation machinery actuator element to express the desired transgene. To assist the secretion of IFNs, the natural signal peptide of the two IFNs was used. Variable domains, which can be exchanged to reprogram the specificity of the effector cell against different disease indications, include an antigen-binding camelid-derived single domain heavy chain (VHH-72; PDB: 6WAQ) as the binding domain of the CAR which has specificity towards SARS-CoV-1 Sgp and cross-reactivity with SARS-CoV-2 Sgp (See FIGS. 10A-10B). To reprogram the effector cell for desired specificity, the VHH-72 sequence can be replaced by an appropriate VHH or variable heavy-light [V_{H}-V_{L}] portion of the scFv. Upon engaging the Sgp, the Sgp-specific effector cell mobilizes the transcriptional machinery of the T-cell to synthesize Type-I (IFN-α2b; IFN-β1a) or Type-III (IFN-λ2; IFN-λ1) IFNs (e.g., the effector proteins) that exert prophylactic or therapeutic effects.

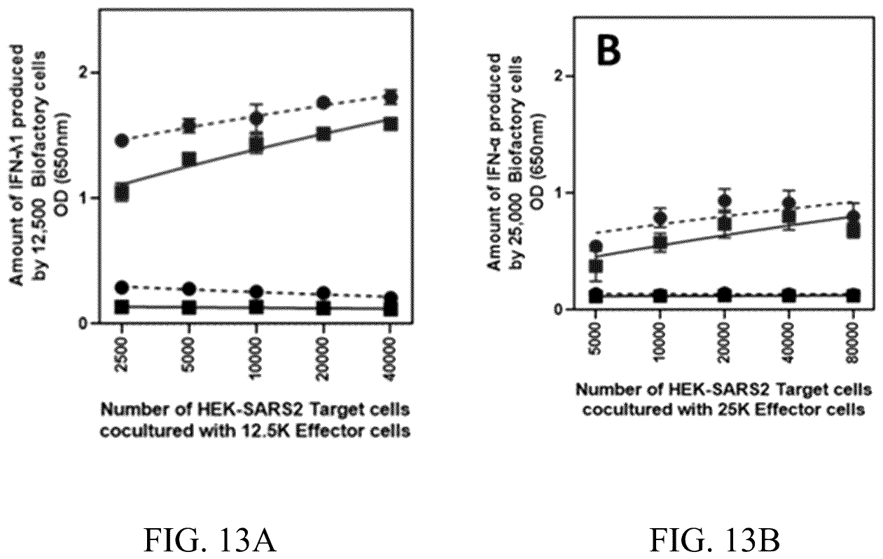

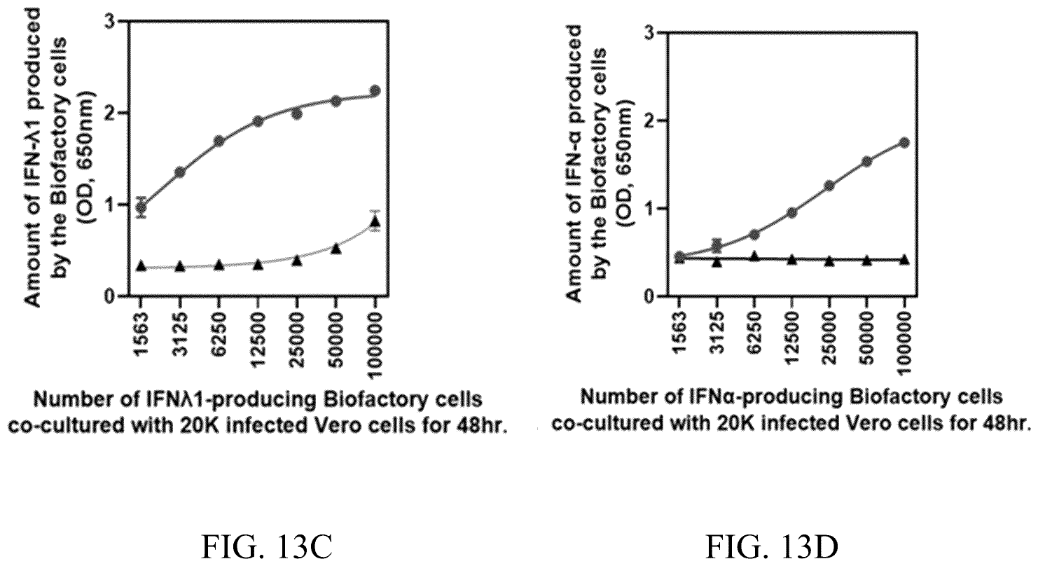

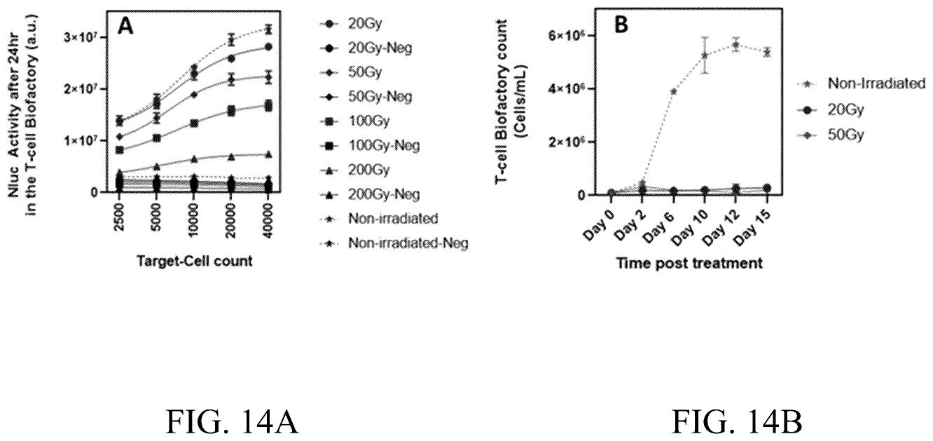

Referring back to FIG. 2, FIG. 2 shows an example schematic of the Sgp-specific effector cell 200 and the potential to identify the SARS-CoV-2-specific Sgp independent of presentation in the peptide-major histocompatibility complex (pMHC). The SARS-CoV-2-specific function of the Sgp-specific effector cell was initially assessed using an genetically engineered target cell that is artificially or pseudo-infected, which is prepared by engineering HEK293T/17 cell line to stably express the Sgp from SARS-CoV-2 (SARS-CoV-2-Sgp-cell) while the non-engineered parental cell line was used as the uninfected negative control. Two classes of the Sgp-specific effector cells were prepared that produced different effector proteins (i) Type-I (IFN-α2b; IFN-β1a), and (ii) Type-III (IFN-λ2; IFN-λ1). Comparison of the two Type-I IFNs (IFN-α2b; IFN-β1a) and two Type-III IFNs (IFN-λ2; IFN-λ1) in context of effector protein production is further illustrated by FIGS. 13A-13D. Based on this comparison, IFN-β1a and IFN-λ1 were used to represent Type-I and Type-III IFNs, respectively. An initial dose assessment of the gamma radiation that does not substantially degrade the artificial cell-signaling pathway but renders the effector cell non-proliferative and non-oncogenic was determined to be at least 20 Gy (FIG. 14A-14B).

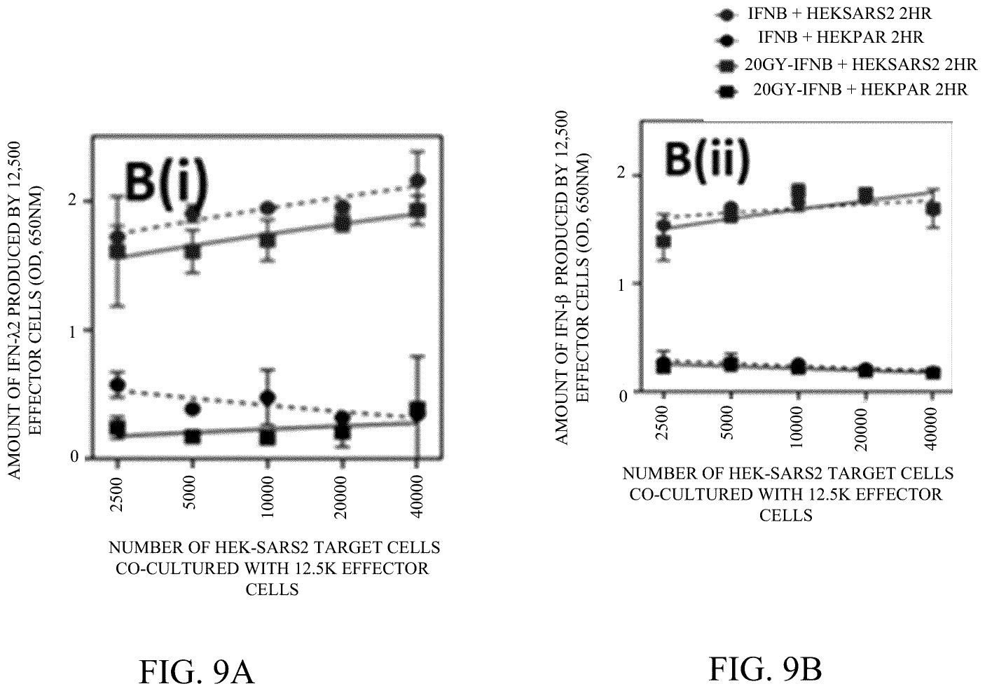

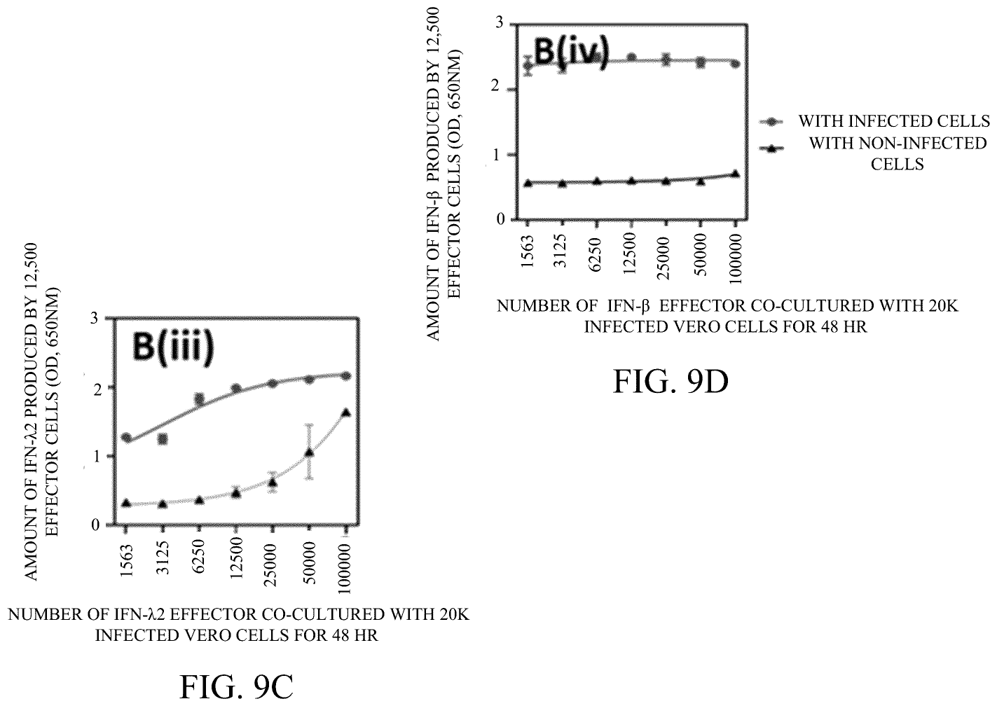

FIGS. 9A-9D illustrate plots characterizing genetically engineered effector cell function, in accordance with the present disclosure. FIGS. 9A-9D illustrate the expression of Type-I and Type-III IFNs in irradiated and non-irradiated effector cells is proportional to the number of target SARS-CoV-2-Sgp cells (e.g., artificial infected cells). FIGS. 9A and 9B show IFN-λ2 and IFN-β production when the effector cell is activated by pseudo-infected cells respectively and the effector cell production is proportional to the target cell count (data collected with effector cell=12,500, 24 hours; Solid lines=Irradiated cells at 20G and Dotted lines=Non-irradiated cells). FIGS. 9C and 9D show for IFN-λ2 and IFN-β in-situ production respectively by the effector cell when stimulated by SARS-CoV-2-infected Vero-E6 cells, and effector proteins production is proportional to the number of effector cell count (data collected with Vero-E6 cells=20,000; MOI=0.05; 48 hours). IFN expression for all observations was measured using n=3, error bars indicate ±1 standard deviation (SD), and can also be considered as one half-width of a 68% confidence interval for the mean.

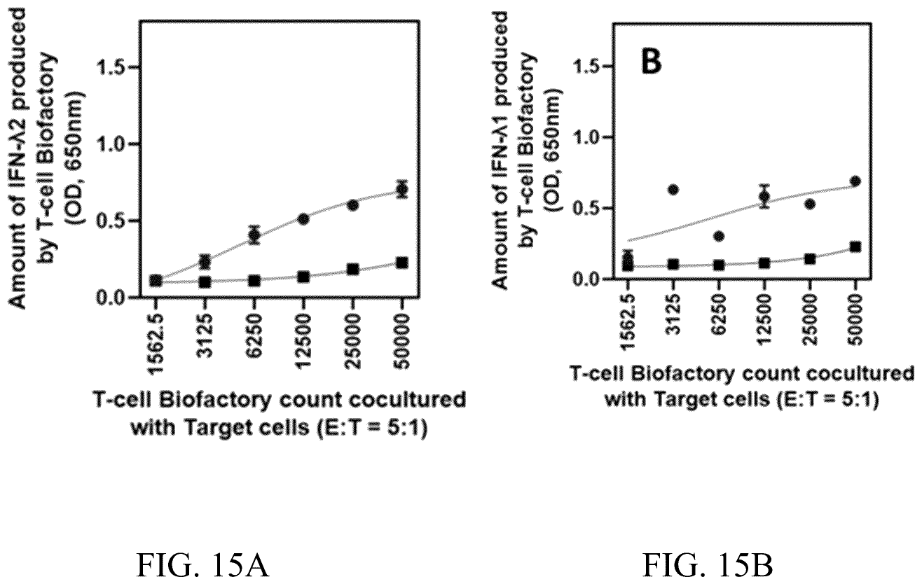

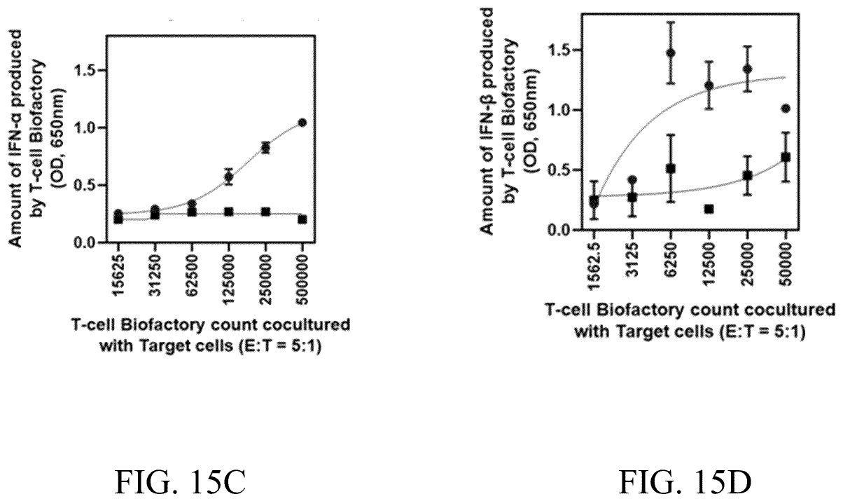

More particularly, FIGS. 9A-9B demonstrate the expression of Type-I IFNs (IFN-β1a) and Type-III IFNs (IFN-λ2) by the effector cells formed using T-cells respectively and compares it with the IFNs produced from the two T-cell types irradiated at 20 Gy. The effector protein (IFNs) expression was proportionate to target cell count and was observed at all effector cell to target cell (E:T) ratios. The expression was significantly elevated (p<0.00001 at all E:T) when stimulated by the target SARS-CoV-2-Sgp-cell compared to when stimulated by the non-engineered negative control cells. Similar results were observed when the two types of T-cell-based effector cells were irradiated with 20 Gy (p<0.0006 at all E:T). To validate the observations from effector cells formed using T-cells with genetically modified target cell, e.g., an artificial or pseudo-infected SARS-CoV-2-Sgp-cell, the target SARS-CoV-2-Sgp-cell was exchanged with the Vero-E6 host cells infected with competent SARS-CoV-2 virus (Isolate: Hong Kong/VM20001061/2020) in BSL3 containment facility. The results presented in FIGS. 9C-9D demonstrate the Type-I IFN (IFN-Ola) and Type-III IFN (IFN-λ2) expression by the respective Sgp-specific effector cell, which was significantly higher when compared the IFN expression from the Sgp-specific effector cell stimulated by uninfected Vero-E6 cells (p<0.0002 for IFN-β1a and p<0.0099 for IFN-λ2 at all E:T ratios). FIGS. 15A-15D depict similar observations from the different Sgp-specific effector cells upon engaging the genetically modified target cell, a pseudo-infected cell-based model of presenting SARS-CoV-1-specific Sgp (SARS-CoV-1-Sgp-cell).

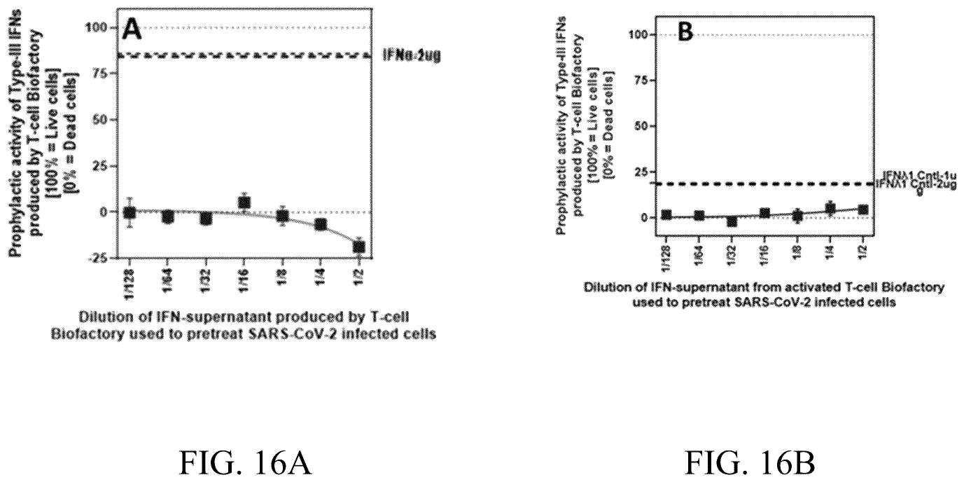

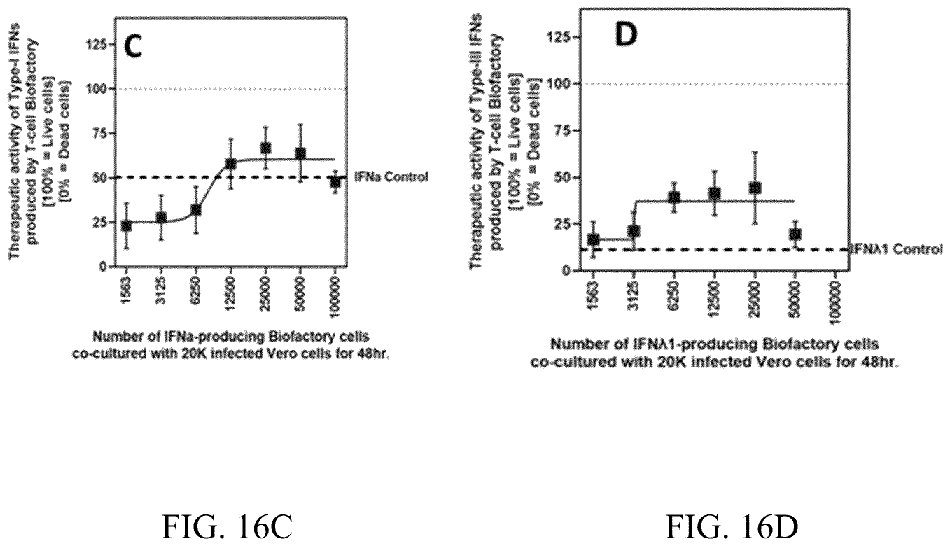

FIGS. 10A-10D illustrate plots characterizing therapeutic activity of example genetically engineered effector cells, in accordance with the present disclosure. Various experiments were directed to assessing the effects of the Type-I IFNs (IFN-β1a) and Type-III IFNs (IFN-λ2) effector cells. To simulate the role of timing in infection, the relative protection offered by the two IFNs before and after the viral challenge was investigated, e.g., their prophylactic and therapeutic effects. Toward this goal, either i) Vero-E6-Luc2⁺ host cells were pretreated with the supernatants from the respective effector cells (IFN-β1a or IFN-λ2) before infecting them with SARS-CoV-2, e.g., prophylaxis effect, or ii) co-cultured the two effectors cells with previously infected Vero-E6-Luc2⁺ host cells, e.g., therapeutic effect. The protection offered by the two effector cells was determined by assessing the live host cells. Protective effects from the other effector cells (type-I IFN-α2b and type-III IFN-λ1) were also measured and are reported in FIGS. 16A-14D.

The IFN producing effector cell can be used as prophylaxis or therapeutic to protect the SARS-CoV-2 infected Vero-E6 cell population. The effect is proportional to the amount of Type-I and Type-III IFNs produced by the effector cell. Activity of the IFN-λ2-T-cell effector cell is shown by FIGS. 10A and 10C. Activity of the IFN-β-T-cell effector cell is shown by FIGS. 10B and 10D. The data was collected with Vero-E6 cells=20,000; MOI=0.05; 48 hr. 1 μg of recombinant human IFN was used as control in all experiments (dashed black line). Red dotted line=100% viability; Black dotted line=0% viability. All observations were measured using n=3, error bars indicate ±1 SD and can also be considered as one half-width of a 68% confidence interval for the mean.

Data in FIGS. 10A-10B illustrate the prophylactic activity of Type-I IFNs (IFN-β1a) and Type-III IFNs (IFN-λ2) produced by respective activated T-cell based effector cells, when compared to the non-activated effector cells. Although Type-III IFNs show reduced protection of host cells (FIG. 10A), cell viability was significantly higher at all concentrations above 0.587 ng/mL, e.g., diluted less than 1/32 (p<0.002), compared to the negative control. Similarly, improved protection was observed by Type-I IFNs when present more than 0.009 ng/mL (p<0.001) (FIG. 10B), compared to the negative control. Results presented in FIGS. 8C-8D demonstrate the therapeutic activity of Type-I IFN (IFN-β1a) and Type-III IFN (IFN-λ2) T-cell-based effector cell when co-cultured with infected host cells. The protection offered by the two effector cells (or amount of IFNs) as a therapeutic for infected host cells was proportional to their number (see FIG. 10C for IFN-λ2; and FIG. 10D for IFN-β1a). This prophylactic and therapeutic activity of IFNs confirm that the SARS-CoV-2 virus is susceptible to IFN treatment and demonstrates the potency of the IFNs produced by the respective effector cell types in reducing viral replication.

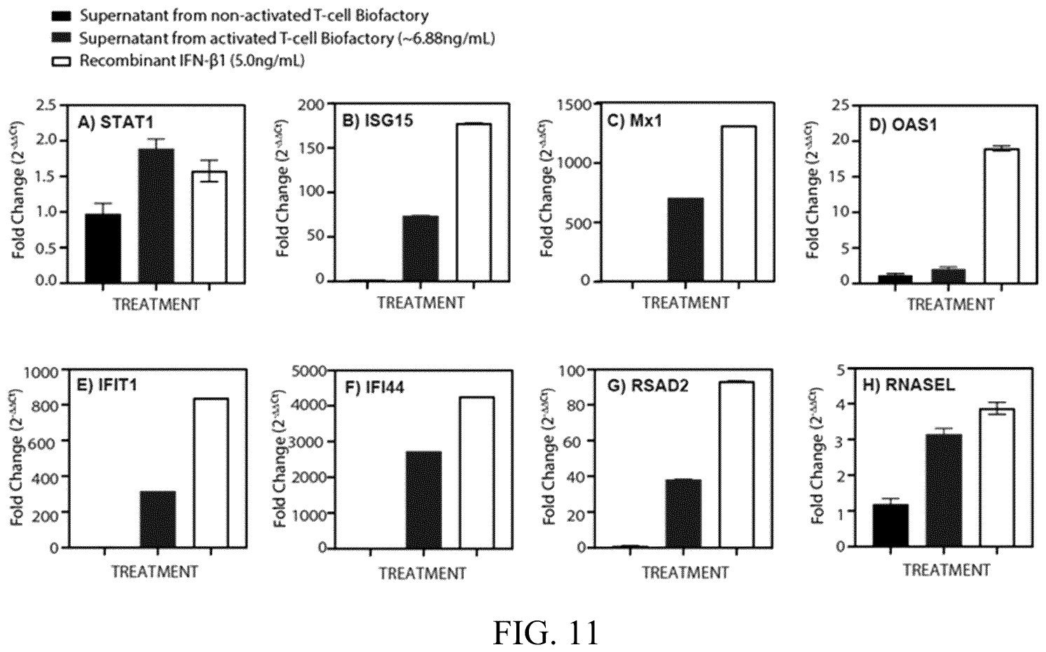

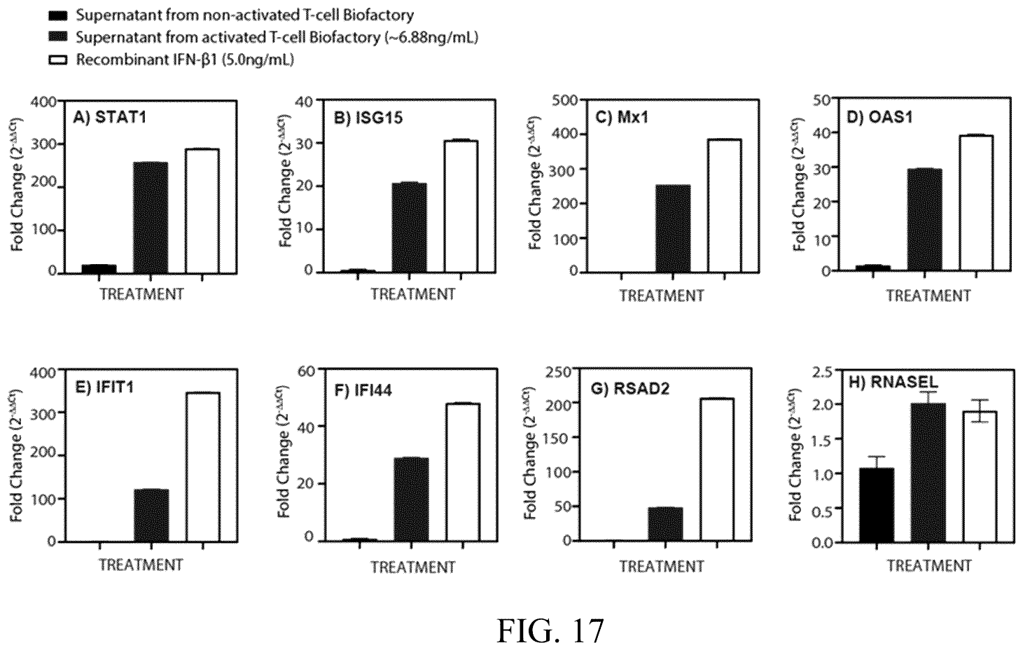

FIG. 11 illustrates plots characterizing IFN signaling of example genetically engineered effector cells, in accordance with the present disclosure. FIG. 11 demonstrates the expression profiles of representative IFN-stimulated genes (ISGs) induced by IFN-β1a from the activated effector cells. Many ISGs control viral spread by directly targeting pathways and functions critical to the viral replication cycle. Expression of Mx1 can inhibit viral entry; ISG15, OAS-RNASEL and IFIT proteins are all reported as inhibitors of viral replication and translation; while upregulation of RSAD2 inhibits viral budding/egress. The induction of ISGs and their downstream production of antiviral effects in the treated host cells was assessed. Both Type-I and Type-III IFNs induced ISG transcription through the Janus Kinase-Signal Transducers and Activators of Transcription (JAK-STAT) signaling pathway, and their expression from the activated effector cells explains the antiviral effects of IFNs observed in FIGS. 10A-10D. The results demonstrate that transcription of most antiviral effector genes (or ISGs) was significantly upregulated in infected Vero-E6 cells treated with supernatants from the activated IFN-β1 producing effector cell, compared to the negative controls, e.g., infected cells treated with supernatants from the unstimulated IFN-β1 producing effector cell (FIG. 11). Similar expression profiles were observed when the supernatant from IFN-β1 producing effector cells were used to treat Calu-3 human epithelial cells (FIG. 17).

FIG. 11 shows the IFN signaling in Vero-E6 cells treated with type-I IFN-β1 from activated effector cells. Vero E6 cells were incubated with the indicated amounts of Type-I IFN supernatants from activated effector cells for 24 hours and then analyzed for expression of ISGs. Error bars represent standard error means (SEM) from three biological replicates.

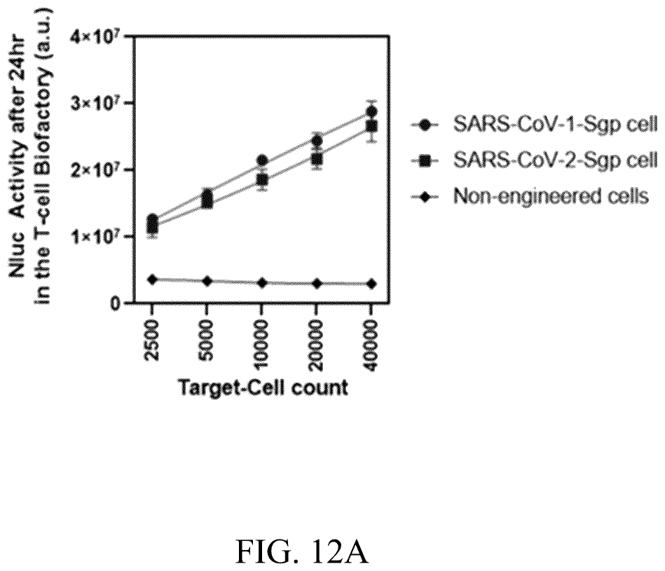

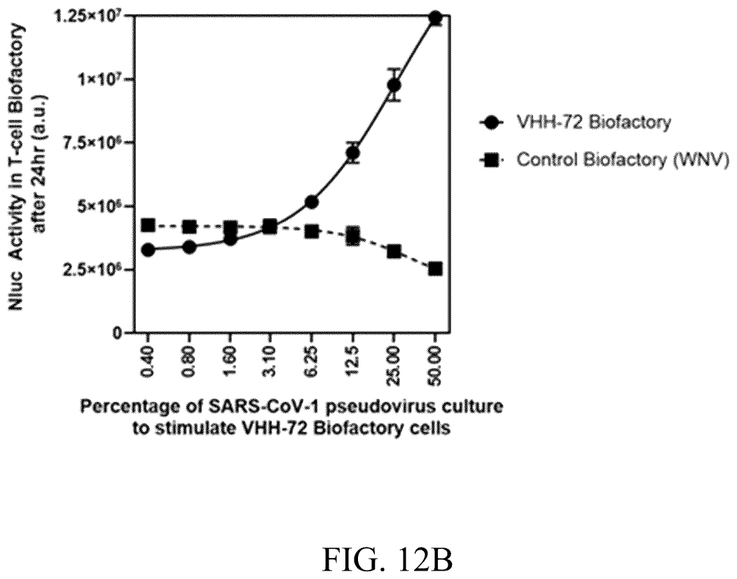

FIGS. 12A-12B illustrate plots characterizing activation and growth of example genetically engineered effector cells, in accordance with the present disclosure. More particular, FIGS. 12A-12B show specificity of the VHH-72 effector to SARS-CoV-1 and SARS-CoV-2. FIG. 12A shows that the Nluc protein expression is proportional to the number of SARS-CoV-2-Sgp-cells or SARS-CoV-1-Sgp-cell target cells (effector cell=12,500, 24 hour). FIG. 12B shows the Nluc activity in the VHH-72 effector cell (50,000) increased with respect to the number of SARS-CoV-1 pseudovirus particles present in solution. Nluc expression for all observations was measured using n=4, error bars indicate ±1 SD, and can also be considered as one half-width of a 68% confidence interval for the mean.

FIGS. 13A-13D illustrate plots characterizing activation of example genetically engineered effector cells by SARS-CoV-2-infected cells, in accordance with the present disclosure. More particularly, FIGS. 13A-13D show IFN production in the Sgp-specific effector cells. FIGS. 13A and 13B respectively illustrate IFN-λ1 and IFN-u production when the effector cell is activated by pseudo-infected cells and its production is proportional to the target cell count (data collected with effector cell=12,500, 24 hours; Solid lines=Irradiated cells at 20G and Dotted lines=Non-irradiated cells); while C) and D) show for IFN-λ1 and IFN-α in-situ production by the effector cell when stimulated by SARS-CoV-2-infected Vero-E6 cells, and production is proportional to the number of effector cell count (data collected with Vero-E6 cells=20,000; MOI=0.05; 48 hours). IFN expression for all observations was measured using n=3, error bars indicate ±1 SD, and can also be considered as one half-width of a 68% confidence interval for the mean.

FIGS. 14A-14B illustrate plot characterizing activation and growth curves of example genetically engineered effector cells, in accordance with the present disclosure. FIGS. 14A-14B more particularly show an activation and growth curve of the irradiated Sgp-specific effector cells. FIG. 14A shows the Nluc protein expression in the effector cell is proportional to the number of target cells. Nluc activity for all observations was measured using n=4, error bars indicate ±1 SD and can also be considered as one half-width of a 68% confidence interval for that mean (blue line=SARS-CoV-2-Sgp-cell targets; black line=non-engineered targets; solid line=Irradiated effector cell; dotted line=Non-irradiated effector cell; effector cell=12,500). FIG. 14B shows the growth kinetics of the irradiated effector cell, at different radiation doses, versus the non-irradiated effector cell.

FIGS. 15A-15D illustrate plots characterizing prophylactic and therapeutic activating of example genetically engineered effector cells, in accordance with the present disclosure. More particularly, FIGS. 15A-15D illustrated VHH-72 effector cell activation by target SARS-CoV-1-Sgp-cell. Expression of Type-I and T-III Interferons (IFNs) in the VHH-72 effector cell is proportional to the effector cell count, IFN-λ2 (as shown by FIG. 15A), IFN-λ1 (as shown by FIG. 15B), IFN-α (as shown by FIG. 15C), and IFN-β (as shown by FIG. 15D) (data collected at 24 hours). IFN expression for all observations was measured using n=3, error bars indicate ±1 SD and can also be considered as one half-width of a 68% confidence interval for the mean.

FIGS. 16A-16D illustrate plot characterizing activation of example genetically engineered effector cells, in accordance with the present disclosure. Prophylactic and therapeutic activity of IFNs produced by the Sgp-specific effector cell are illustrated by FIGS. 16A-16D. The viability of SARS-CoV-2 infected Vero-E6 cells is proportional to the amount of Type-I and Type-III IFNs produced by the effector cell. FIGS. 16A and 16C show activity of the IFN-λ1-effector cell, and FIGS. 16B and 16D show activity of the IFN-α-effector cell (data collected with Vero-E6 cells=20,000; MOI=0.05; 48 hour). 1 μg of recombinant human IFN was used as a control in all experiments (dashed black line). Red dotted line=100% viability; black dotted line=0% viability. All observations were measured using n=3, error bars indicate ±1 SD and can also be considered as one half-width of a 68% confidence interval for that mean.

FIG. 17 illustrates plots characterizing IFN signaling of example genetically engineered effector cells in response to SARS-CoV-2-infected cells, in accordance with the present disclosure. More particularly, FIG. 17 shows IFN signaling in Calu-3 cells treated with Type-I IFNs from activated effector cells. Calu-3 cells were incubated with the indicated amounts of Type-I IFN supernatants from activated effector cells for 24 hours and then analyzed for expression of ISGs. Error bars represent SEM from three biological replicates.

Synthesis and Experimental Information for Effector Cell Specific to Sgp

(1) Materials and reagents. Engineered Jurkat E6-1 (ATCC, Cat #TIB-152) cell lines were maintained in complete RPMI media (RPMI1640 [Corning, Cat #10-040-CV], 10% were heat-inactivated fetal bovine serum or FBS [Sigma-Aldrich, Cat #F2442-500ML] and 1× Penicillin-Streptomycin solution [Corning, Cat #30-002-Cl]). Parental and Engineered Vero-E6 cells (ATCC, Cat #CRL-1586) were cultured in complete EMEM (EMEM growth media [Corning, Cat #10-009-CV] supplemented with 10% heat-inactivated FBS and 1× Penicillin-Streptomycin solution). Parental and Engineered HEK293T/17 cells (ATCC, Cat #CRL-11268) were cultured in complete DMEM (DMEM growth media [Corning, Cat #10-013-CV] supplemented with 10% FBS and 1× Penicillin-Streptomycin solution). All cells were expanded, and liquid nitrogen stocks were maintained using freezing media (50% FBS, 40% growth media and 10% Dimethyl sulfoxide). Plasmids encoding different genetic payloads (transfer plasmids) were designed in SnapGene software (GSL Biotech LLC) and sub-cloned into lentivirus vector plasmid (System Biosciences, Cat #CD510B-1) or PiggyBac Transposon vector plasmid (System Biosciences, Cat #PB510B-1). Plasmids encoding 2nd generation packaging plasmids (psPAX2—Cat #12260, pMD2.G—Cat #12259) were obtained from Addgene. pAdvantage was obtained from Promega (Cat #E1711). PiggyBac Transposase sequence was provided by the Johns Hopkins University School of Medicine [Doherty, J. E. et al. Hyperactive piggyBac gene transfer in human cells and in vivo. Human Gene Therapy 23, 311-320 (2012).] An insert for “EF1alpha promoter—i7pB transgene—bGH poly(A) signal” was chemically synthesized and assembled using overlapping PCR products into pUC19 (GenBank: L09137, New England Biolabs, #N3041). All plasmid preparation services (chemical synthesis of DNA insert sequences, sub-cloning into respective vector backbones, and the amplification) were obtained from Epoch Life Science, Inc. (Missouri City, TX). For lentivirus production, Transporter 5™ reagent (Polysciences, Inc, Cat #26008-5) was used to transfect parental HEK293T/17 cells. The collected lentivirus was transduced into Jurkat cells using Polybrene (Abm®, Cat #G062). TransIT®-2020 transfection reagent (Mirus #MIR5400) was used to transfect PiggyBac Transposon system plasmids into parental HEK293T/17 cells to engineer stable antigen-presenting cells (APCs) or pseudo-infected host target cells. Puromycin dihydrochloride (ThermoFisher Scientific, Cat #A1113803) was used for selecting stable cells. Phosphate buffered saline (PBS) without Ca⁺² and Mg⁺² (Corning, Cat #21-040-CV) was used to wash cells. The SARS-CoV-2 virus culture (BEI Resources, NIH; Hong Kong/VM20001061/2020 [Cat #NR-52282]) was provided SRI International. Viability of Vero-E6-Luc2⁺ cells was assessed using either CellTiter-Glo™ Luminescent Cell Viability Assay Kit (Promega, Cat #PR-G7570) or One-Glo® assay (Promega, Cat #E6110) for Luc2 activity. HEK-Blue IFN-α/β cells (InvivoGen, Cat #hkb-ifnab) or HEK-Blue IFN-λcells (InvivoGen, Cat #hkb-ifn1) were used to quantify the amount of IFNs produced by the T-cell Biofactory, following manufacturer's instructions. Recombinant human interferon proteins from R&D systems (IFN-0 [Cat #8499-IF-010/CF], IFN-λ1 [Cat #1598-IL-025/CF], IFN-λ2 [Cat #8417-IL-025/CF]) and IFN-α2b from Invivogen [Cat #rcyc-hifna2b] were used as controls. For RNA preparations, Direct-zol RNA Mini-Prep Kit (Zymo Research, Cat #11-331) and reverse transcription by SuperScript III RT (Invitrogen, Cat #18080044) were used following the manufacturers' instructions. TaqMan Universal PCR Master Mix (Applied Biosystems, Cat #4305719) was used for gene expression qPCR.

(2) Lentivirus production. Lentivirus particles were prepared by packaging the transfer plasmid using 2^{nd} generation lentivirus system as detailed previously by Radhakrishnan H, et al. (2020); Lentivirus Manufacturing Process for Primary T-cell Biofactory Production. Advanced Biosystems, 1900288, which is hereby incorporated herein in its entirety for its teaching.

(3) Generation of the Sgp-specific effector cell (T-cell Biofactory). The Jurkat E6-1 suspension cell line was engineered with lentivirus particles carrying the genetic payload (FIG. 2) with a VHH-72 (PDB: 6WAQ) CAR or sensor domain, as detailed previously by Radhakrishnan H, et al. (2020), entitled “Lentivirus Manufacturing Process for Primary T-cell Biofactory Production. Advanced Biosystems”, 1900288, which is hereby incorporated herein in its entirety for its teaching. The cells were treated with lentivirus in the presence of 8 μg/mL Polybrene. After 48 hours, the engineered effector cells were placed in selection using 0.5 μg/mL of Puromycin dihydrochloride. The unmodified parental cell line was also placed under selection as a positive control for cell killing by Puromycin. Following selection, cells were expanded as required for different assays and frozen using freezing media. For irradiation, the Sgp-specific effector cell was exposed to 20 Gy (or as indicated) using a 137Cs γ-emitting irradiator, Mark I-68A (JL Shepherd and Associates) at a dose rate of 222 mGy/min. The control cells were treated similarly except for the irradiation.

(4) Generation of genetically modified target cells (SARS-CoV-2-Sgp-cell and Vero-E6-Luc2⁺). Parental HEK293T/17 were engineered to stably express Sgp (SARS-CoV-2-Sgp-cell) while parental Vero-E6 cells were engineered to express intracellular Luc2 reporter protein (Vero-E6-Luc2⁺), using the PiggyBac Transposon system as previously described by Repellin C E, et al. (2020); Engineered Ovarian Cancer Cell Lines for Validation of CAR T-cell Function. Advanced Biosystems, 1900224. The plasmids were designed with the PiggyBac transposon vector backbone for cell surface expression of Sgp from SARS-CoV-2 (GenBank: QHD43416.1) and for expression of Luc2 Reporter transgene (GenBank: AY738222.1) respectively. A monolayer of HEK293T/17 or Vero-E6 cells were transfected with the transposon plasmid (carrying the gene of interest) and transposase plasmid, in a ratio of 2.5:1, respectively, using TransIT®-2020 transfection reagent. After 48 hours of transfection, the transfected cells were placed under selection using 0.5 μg/mL (or 3 ug/mL for Vero-E6-Luc2⁺) of Puromycin dihydrochloride. The unmodified parental cell lines were placed under selection as a positive control for cell killing by the antibiotics. The generated stable cell lines were expanded as required for different assays.

(5) Co-culture of Sgp-specific effector cell (T-cell Biofactory) with target SARS-CoV-2-Sgp-cell (genetically modified target cell). Target SARS-CoV-2-Sgp-cell or non-engineered cells were co-cultured at different effector cell to target cell (E:T) ratios with the Sgp-specific effector cell in 100 μL of complete RPMI media in a single well of a 96-well plate. After the specified amount of time in co-culture, the original amount of IFNs produced by the effector cell was assessed using the HEK-Blue IFN-α/β or IFN-λreporter assays, following the manufacturer's protocol. Similar co-culture procedures were used when testing the irradiated Sgp-specific effector cells.

(6) Determining the prophylactic effect of IFNs produced by the Sgp-specific effector cells. 2×10⁵ of target SARS-CoV-2-Sgp-cell or non-engineered cells were co-cultured with 1×10⁶ of the Sgp-specific effector cells (effector cell to target cell ratio, E:T=5:1) in 0.5 mL of complete RPMI media in a single well of a 24-well plate. After the specified amount of time in co-culture, supernatants with IFNs were collected and serially diluted in complete EMEM media. 100 μL of serially diluted IFN-supernatants (2-fold) was then used to pre-treat a monolayer of Vero-E6-Luc2⁺ cells for 24 hours (20,000 cells/well, triplicates) in a 96-well plate; recombinant human IFNs (1 μg/mL) were included as controls. The pre-treated cells were then infected with SARS-CoV-2 virus culture at a multiplicity of infection (MOI) of 0.05 for 48 hours. Cell viability was determined using CellTiter-Glo™ Luminescent Cell Viability Assay Kit following the manufacturer's instructions. The enzyme substrate (Nluc substrate or Luc2 substrate) was diluted in the cell lysis buffer provided with the Nano-Glo® or One-Glo® assay and added to the co-cultures in a 96-well plate for assessing enzyme (Nluc or Luc2) activity. Following a brief incubation period (3 minutes for Nluc or 10 minutes for Luc2), bioluminescence was read on a microplate reader (Perkin Elmer, EnVision™ Multilabel Plate Reader Model: 2104-0010A). The original amount of IFNs produced by each effector cell was assessed using the HEK-Blue IFN-α/β or IFN-λreporter assays as described below. ATP activity was normalized, where 100%=No IFN or virus treatment used and 0%=only virus infection.

(7) Determining the therapeutic effect of IFNs produced by the Sgp-specific effector cell. A monolayer of Vero-E6-Luc2⁺ (20,000/well in a 96-well plate) were infected with SARS-CoV-2 virus culture at an MOI of 0.05 and incubated for 2 hours to allow virus attachment. After 2 hours, the virus inoculum was removed, and 150 μL of serially diluted IFN-producing effector cell (2-fold) was immediately added to the wells (triplicates). Recombinant human IFNs (1 μg/mL) and Non-infected Vero-E6-Luc2⁺ cells were used as controls. After 48 hours of co-culture, 50 μL of supernatant was removed from each well to quantify the amount of IFNs produced by the effector cell at each E:T ratio, using the HEK-Blue IFN-α/D or IFN-λreporter assays. Then, Vero-E6-Luc2⁺ cell viability was determined by assessing Luc2 activity using the One-Glo® assay kit, following the manufacturer's instructions. The Luc2 enzyme substrate was diluted in the cell lysis buffer provided and added to the cells in 96-well plate for assessing Luc2 enzyme activity. Following a 10-minute incubation, bioluminescence was read on a microplate reader. Luc2 activity was normalized, where 100%=No IFN or virus treatment used and 0%=only virus infection.

(8) HEK-Blue IFN-α/β and IFN-λreporter assays. Following the manufacturer's instructions, 150 μl of growth media containing 50,000 HEK-Blue IFN-α/β and HEK-Blue IFN-λreporter cells were mixed with 50 μL of supernatant from the activated IFN-producing effector cell and plated in a single well of a 96-well plate. Serial dilutions of Type-I or Type-III IFNs in complete DMEM were added in parallel to generate a standard curve. After 24 hours of incubation, 20 μL of HEK-Blue IFN-α/β (or HEK-Blue IFN-λ) supernatants were added to 180 μL of Quanti-blue substrate (InvivoGen) and incubated at 37° C. for 2 hours. Absorbance was measured at 650 nm using an Envision microplate reader (Perkin-Elmer). The standard curves were used to estimate the IFN concentrations produced by the Sgp-specific effector cell.

(9) Quantification of IFN-stimulated gene (ISG) mRNA expression. IFN-β1a supernatants from stimulated effector cell (diluted at 1/4 dilution or ˜6.88 ng/mL) were used to treat a monolayer of 2×10⁵ Vero-E6 (or Calu-3) cells (in triplicate) for 24 hr (5 ng/mL of recombinant IFN-β1a was used as control). 300 uL of TRIzol was used to lyse cells and then RNA purifications were performed using Direct-zol RNA Miniprep kit following manufacturer's instructions. Purified RNA was reverse transcribed into cDNA using the SuperScript™ III Reverse Transcriptase. The cDNAs were analyzed by qPCR using TaqMan Universal PCR Master Mix and TaqMan gene expression assays. The following TaqMan primer/probe sets were used to assess type-I IFN signaling: GAPDH (Hs02786624_g1), 18S (Hs99999901_s1), ACTB (Hs03023880_g1) IFIT1 (Hs03027069_s1), IF144 (Hs00951348 ml), STAT1 (Hs00234829_ml), ISG15 (Hs01921425_s1), OAS1 (Hs05048921_s1), RNASEL (Hs05030865_s1), RSAD2 (Hs04967697_s1), and MX1 (Hs00895608_ml). All qPCR was performed in 384-well plates and run on a ViiA7 real time PCR system (Cat #4453545). ISG expression was calculated using the ΔΔCT method⁴³ by normalizing the threshold cycle (Ct) values to reference genes (GAPDH, 18S and ACTB), and expressions are represented as fold changes over untreated cell samples. Error bars represent standard error means (SEM) from the three biological replicates.

(10) Experimental designs and statistical analysis. The experimental design for each panel in the figures is described below. GraphPad Prism 9.2.0 (GraphPad Software, Inc) was used to conduct all statistical analyses.

(i) FIG. 9A and FIG. 9B are directed to IFN production by the irradiated and non-irradiated effector cells when activated by pseudo-infected target cells. The IFN production in the Sgp-specific effector cells when stimulated by the target (SARS-CoV-2-Sgp-cell) or non-target (non-engineered) cells was fitted using the equation Y=a+b*X; where Y is the IFN amount produced, X is the Log₁₀ (target cell count), a is the Y-intercept and b is the slope of the fitted curve.

(ii) FIG. 9C and FIG. 9D are directed to IFN production by the effector cell when activated by virally infected target cells. The IFN production in the Sgp-specific effector cell when stimulated by the infected target or non-infected target cells was fitted using the using a four-parameter logistic model, IFN=IFNₘᵢₙ+[[[IFNₘₐₓ−IFNₘᵢₙ}/[[[1+10\hat{ }[b*(log₁₀[Effector-Cell₅₀]−X)]}; where X is the log₁₀ of the effector cell (T-cell Biofactory) count, IFNₘₐₓ is an estimated parameter defining a upper asymptote for IFN production, IFNₘᵢₙ is an estimated parameter defining a lower asymptote for IFN production, b is a “Hill” parameter defining the slope at the inflection point of the fitted curve, and Effector-Cell₅₀ is an estimated parameter representing the X value corresponding to (IFNₘₐₓ−IFNₘᵢₙ)/2.

(iii) FIG. TOA and FIG. 10B are directed to demonstrating the prophylactic activity of IFNs produced by the effector cell. Comparison of all data points was calculated by the false discovery rate (FDR) multiple comparison approach, using the two-stage step-up procedure of Benjamini, Krieger, and Yekutieli with FDR=1%. There was no adjustment for multiple comparisons. The error bars extend 1 SD above and below the mean and can also be considered as one half-width of an 68% confidence interval for that mean. The ATP activity was normalized and fitted using a four-parameter logistic model ATP=ATPₘᵢₙ+[[[ATPₘₐₓ−ATPₘᵢₙ}/[[[1+10\hat{ }[b*(log₁₀[Dilution₅₀]−X)]}; where X is the log₁₀ of the IFN-dilution, ATPₘₐₓ is an estimated parameter defining a upper asymptote for ATP activity, ATPₘᵢₙ is an estimated parameter defining a lower asymptote for ATP activity, b is a “Hill” parameter defining the slope at the inflection point of the fitted curve, and Dilution₅₀ is an estimated parameter representing the X value corresponding (ATPₘₐₓ−ATPₘᵢₙ)/2.

(iv) FIG. 10C and FIG. 10D are directed to demonstrating the therapeutic activity of IFNs produced by the effector cells. Comparison of all data points was calculated by the false discovery rate (FDR) multiple comparison approach, using the two-stage step-up procedure of Benjamini, Krieger, and Yekutieli with FDR=1%. There was no adjustment for multiple comparisons. The error bars extend 1 SD above and below the mean and can also be considered as one half-width of a 68% confidence interval for that mean. The Luc2 activity when the effector cell was co-cultured with infected or non-infected Vero-E6-Luc2+ target cells was fitted using a four-parameter logistic model, Luc2=Luc2 min+[[[Luc2max−Luc2 min}/[[[1+10\hat{ }[b*(log 10[η(E:T)₅₀]−X)]}; where X is the log 10 of the number of Effector-Cells (T-cell Biofactory), Luc2max is an estimated parameter defining an upper asymptote for the Luc2 activity, Luc2 min is an estimated parameter defining a lower asymptote for the Luc2 activity, b is a “Hill” parameter defining the slope at the inflection point of the fitted curve. A parameter for defining the antiviral efficiency of the produced IFNs, η(E:T)₅₀, was determined as the E:T at which Luc2 activity in infected or non-infected target cells was 50% of the difference between the maximum and minimum values of their respective normalized Luc2 activities, when co-cultured with the effector cell, e.g., the η(E:T)₅₀ is an estimated E:T value corresponding to (Luc2max−Luc2 min)/2.

(v) FIG. 11 shows IFN signaling in Vero-E6 cells treated with type-I IFNs from activated effector cells. Differential gene expression was calculated using the ΔΔCT method by normalizing the sample Ct values to mean Ct values of 3 reference genes, and expressions are represented as fold changes over untreated cell samples. Error bars represent SEM from the three biological replicates.

(vi) FIGS. 12A-12B show Sgp-specific effector cell specificity to SARS-CoV-2 and SARS-CoV-1 infections. The Nluc reporter activity in the Sgp-specific effector cell when stimulated by genetically modified target cells (SARS-CoV-2-Sgp-cell and SARS-CoV-1-Sgp-cell) or non-target (non-engineered) cells was fitted using a four-parameter logistic model Nluc=Nlucₘᵢₙ+[[[Nlucₘₐₓ−Nlucₘᵢₙ}/[[[1+10\hat{ }[b*(log₁₀[Target₅₀]−X)]}; where X is the log₁₀ of the target cell count, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and Target₅₀ is an estimated parameter representing the X value corresponding (Nlucₘₐₓ−Nlucₘᵢₙ)/2.

(vii) FIG. 13A and FIG. 13B show IFN production by the irradiated and non-irradiated effector cells when activated by genetically modified target cell, e.g., pseudo-infected target cells. The IFN production in the Sgp-specific effector cell when stimulated by the target (SARS-CoV-2-Sgp-cell) or non-target (non-engineered) cells was fitted using the equation Y=a+b*X; where Y is the IFN amount produced, X is the Log₁₀ (target cell count), a is the Y-intercept and b is the slope of the fitted curve.

(viii) FIG. 13C and FIG. 13D show IFN production by the effector cell when activated by virally infected target cells. The IFN production in the Sgp-specific effector cell when stimulated by the infected target or non-infected target cells was fitted using the using a four-parameter logistic model, IFN=IFNₘᵢₙ+[[[IFNₘₐₓ−IFNₘᵢₙ}/[[[1+10\hat{ }[b*(log₁₀[effector cell₅₀]−X)]}; where X is the log₁₀ of the effector cell (T-cell Biofactory) count, IFNₘₐₓ is an estimated parameter defining a upper asymptote for IFN production, IFNₘᵢₙ is an estimated parameter defining a lower asymptote for IFN production, b is a “Hill” parameter defining the slope at the inflection point of the fitted curve, and effector cell₅₀ is an estimated parameter representing the X value corresponding to (IFNₘₐₓ−IFNₘᵢₙ)/2.

(ix) FIG. 14A shows Nluc activity in irradiated effector cell is proportional to the number of target cell. The Nluc activity in the irradiated or non-irradiated effector cells stimulated by the target (SARS-CoV-2-Sgp-cell) or non-target (non-engineered) cells was fitted using a four-parameter logistic model Nluc=Nlucₘᵢₙ+[[[Nlucₘₐₓ−Nlucₘᵢₙ}/[[[1+10\hat{ }[b*(log₁₀[infected-Target₅₀]−X)]}; where X is the log₁₀ of the infected target cell count, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and infected-Target₅₀ is an estimated parameter representing the X value corresponding (Nlucₘₐₓ−Nlucₘᵢₙ)/2. Multiple t-test comparisons were calculated by the false discovery rate (FDR), using the two-stage step-up procedure of Benjamini, Krieger, and Yekutieli with FDR=1%. There was no adjustment for multiple comparisons. The error bars extend 1 SD above and below the mean and can also be considered as one half-width of a 68% confidence interval for that mean.

(x) FIG. 14B shows growth kinetics of the effector cell. Line graph showing effector cell counts per mL over time (in days).

(xi) FIGS. 15A-15D show IFN production by effector cell activated by SARS-CoV-1 genetically modified target cell, e.g., pseudo-infected target cells. The IFN production in the Sgp-specific effector cell when stimulated by SARS-CoV-1 pseudo-infected or non-engineered target cells was fitted using the using a four-parameter logistic model, IFN=IFNₘᵢₙ+[[[IFNₘₐₓ−IFNₘᵢₙ}/[[[1+10\hat{ }[b*(log₁₀[Effector-Cell₅₀]−X)]}; where X is the log₁₀ of the effector cell (T-cell Biofactory) count, IFNₘₐₓ is an estimated parameter defining a upper asymptote for IFN production, IFNₘᵢₙ is an estimated parameter defining a lower asymptote for IFN production, b is a “Hill” parameter defining the slope at the inflection point of the fitted curve, and effector-cell₅₀ is an estimated parameter representing the X value corresponding to (IFNₘₐₓ−IFNₘᵢₙ)/2.

(xii) FIGS. 16A-16B show prophylactic activity of IFNs produced by the effector cell. Comparison of all data points was calculated by the false discovery rate (FDR) multiple comparison approach, using the two-stage step-up procedure of Benjamini, Krieger, and Yekutieli with FDR=1%. There was no adjustment for multiple comparisons. The error bars extend 1 SD above and below the mean and can also be considered as one half-width of a 68% confidence interval for that mean. The ATP activity was normalized and fitted using a four-parameter logistic model ATP=ATPₘᵢₙ+[[[ATPₘₐₓ−ATPₘᵢₙ}/[[[1+10\hat{ }[b*(log₁₀[Dilution₅₀]−X)]}; where X is the log₁₀ of the IFN-dilution, ATPₘₐₓ is an estimated parameter defining a upper asymptote for ATP activity, ATPₘᵢₙ is an estimated parameter defining a lower asymptote for ATP activity, b is a “Hill” parameter defining the slope at the inflection point of the fitted curve, and Dilution₅₀ is an estimated parameter representing the X value corresponding (ATPₘₐₓ−ATPₘᵢₙ)/2.

(xiii) FIGS. 16C-16D show therapeutic activity of IFNs produced by the effector cells. Comparison of all data points was calculated by the false discovery rate (FDR) multiple comparison approach, using the two-stage step-up procedure of Benjamini, Krieger, and Yekutieli with FDR=1%. There was no adjustment for multiple comparisons. The error bars extend 1 SD above and below the mean and can also be considered as one half-width of a 68% confidence interval for the mean. The Luc2 activity when the effector cell was co-cultured with infected or non-infected Vero-E6-Luc2⁺ target cells was fitted using a four-parameter logistic model, Luc2=Luc2 min+[[[Luc2max−Luc2 min}/[[[1+10\hat{ }[b*(log 10[η(E:T)₅₀]−X)]}; where X is the log 10 of the number of effector cells (T-cell Biofactory), Luc2max is an estimated parameter defining an upper asymptote for the Luc2 activity, Luc2 min is an estimated parameter defining a lower asymptote for the Luc2 activity, b is a “Hill” parameter defining the slope at the inflection point of the fitted curve. A parameter for defining the antiviral efficiency of the produced IFNs, η(E:T)₅₀, was determined as the E:T at which Luc2 activity in infected or non-infected target cells was 50% of the difference between the maximum and minimum values of their respective normalized Luc2 activities, when co-cultured with the effector cell; e.g., the η(E:T)₅₀ is an estimated E:T value corresponding to (Luc2max−Luc2 min)/2.

(xiv) FIG. 17 shows IFN signaling in Calu-3 cells treated with Type-I IFNs from activated effector cell. Differential gene expression was calculated using the ΔΔCT method by normalizing the sample Ct values to mean Ct values of 3 reference genes, and expressions are represented as fold changes over untreated cell samples. Error bars represent standard error means (SEM) from the three biological replicates.

Table 1 below provides difference sequences used to generate the effector cells.

TABLE 1
List of genes used

| GENETIC ELEMENT | SEQ ID NO |
|---|---|
| SARS-CoV-2 Sgp | SEQ ID NO: 1 |
| SARS-CoV-1 Sgp | SEQ ID NO: 2 |
| VHH-72 antibody | SEQ ID NO: 3 |
| Type-III Interferon (IFN-λ2) | SEQ ID NO: 4 |
| Type-III Interferon (IFN-λ1) | SEQ ID NO: 5 |
| Type-I Interferon (IFN-α2b) | SEQ ID NO: 6 |
| Type-I Interferon (IFN-β1a) | SEQ ID NO: 7 |
| Luc2 | SEQ ID NO: 8 |
| E2 Crimson | SEQ ID NO: 9 |
| Luc2-P2a-E2 Crimson | SEQ ID NO: 10 |
| GFP | SEQ ID NO: 11 |
| Nluc | SEQ ID NO: 12 |
| P2a | SEQ ID NO: 13 |
| GFP-P2a-Nluc | SEQ ID NO: 14 |
| Plasmid 1 (control) | SEQ ID NO: 15 |
| Plasmid 2 (SARS-CoV-2 Sgp) | SEQ ID NO: 16 |
| Plasmid 3 (SARS-CoV-1 Sgp) | SEQ ID NO: 17 |
| Plasmid 4 (VHH-72) | SEQ ID NO: 18 |
| Plasmid 5 (SARS-CoV-1 Sgp) | SEQ ID NO: 19 |
| Plasmid 6 (SARS-CoV-1 Sgp) | SEQ ID NO: 20 |
| Plasmid 7 (SARS-CoV-1 Sgp) | SEQ ID NO: 21 |
| Plasmid 8 (SARS-CoV-1 Sgp) | SEQ ID NO: 22 |
| Plasmid 9 (SARS-CoV-1 Sgp | SEQ ID NO: 23 |
| pseudoviral particles) | |
| Plasmid 8 (VHH-72) | SEQ ID NO: 24 |

Other experimental embodiments were directed to transforming T-cells to generated genetically modified diagnostic cells, which can be used to diagnose a host via an antigen test. Specific embodiments where directed to generating diagnostic cell, a type of effector cell, from immortalized human T-acute lymphoblastic leukemia (T-cell) cell line (Jurkat cells) and which are specific to the Sgp of SARS-CoV-2. The Sgp can be expressed by SARS-CoV-2 virions, and when the diagnostic cell encounters Sgp-presenting cells, such as infected host cells or virus particles or virions, the diagnostic cell is activated to express bioluminescent and fluorescent reporter proteins.

Referring back to FIG. 5B, FIG. 5B illustrates an example schematic of the mechanism of the diagnostic cell detecting Sgp antigens on infected cells. The Diagnostic cell uses an artificial cell-signaling pathway composed of two constant and two variable domains in cis, as described above and are shown in FIG. 5C. The constant domains provide functionality to the diagnostic cell and include a transmembrane molecule (receptor) that mobilizes the T-cell activation machinery (actuator) to upregulate the desired transgene. The variable domains include a camelid-derived, VHH binding domain, which is part of the transmembrane receptor element, that upon engaging the antigen biomarker, mobilizes the constant domains to synthesize the reporter proteins encoded by the effector element. Both variable domains can be exchanged to impart broad applicability to the diagnostic cell platform. In experimental embodiments, the VHH was used to develop a diagnostic cell with specificity either for both SARS viruses (SARS-CoV-2 and SARS-CoV-1), or for only SARS-CoV-2 without cross-reactivity to SARS-CoV-1. The reporter proteins used were a dual-reporter protein for fluorescence (GFP) and bioluminescence (NanoLuc® [Nluc], Promega, or GFP-2A-Nluc). A secretor peptide was not needed for the diagnostic cell.

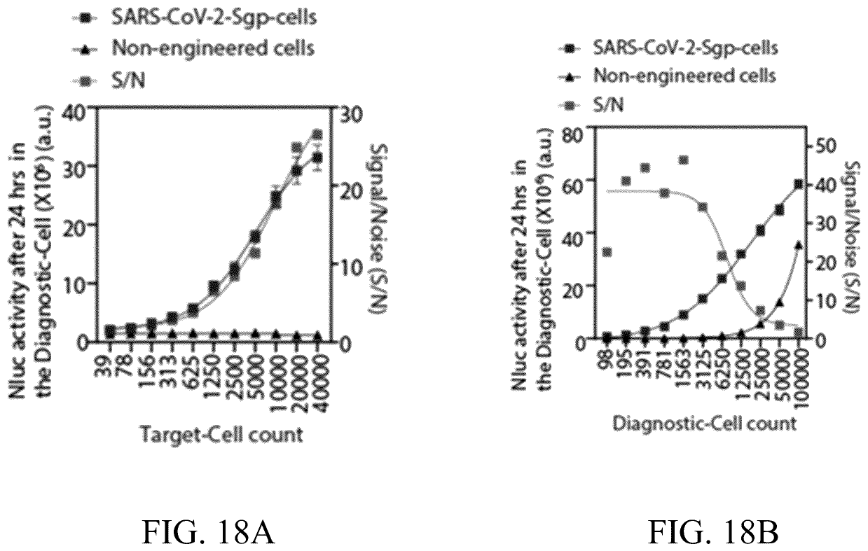

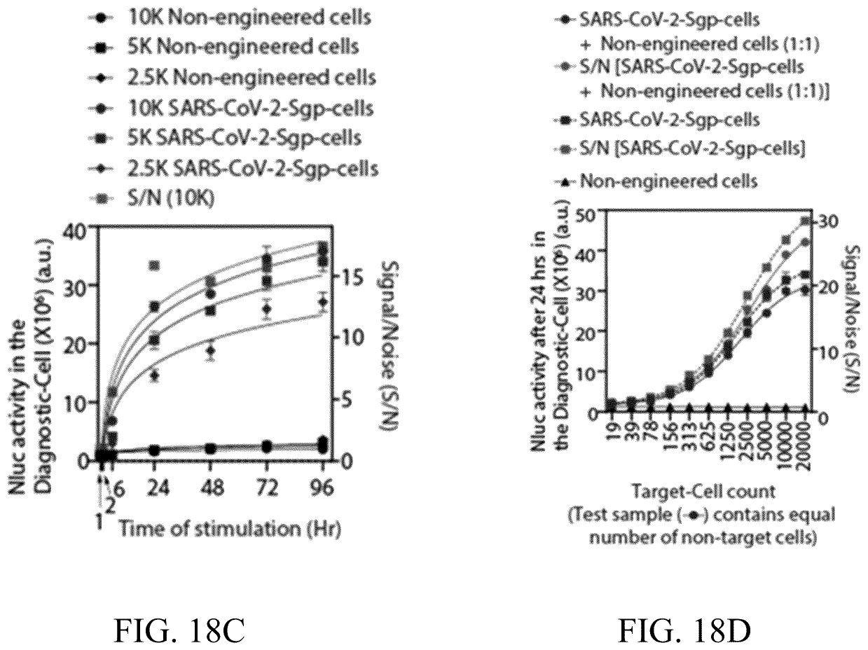

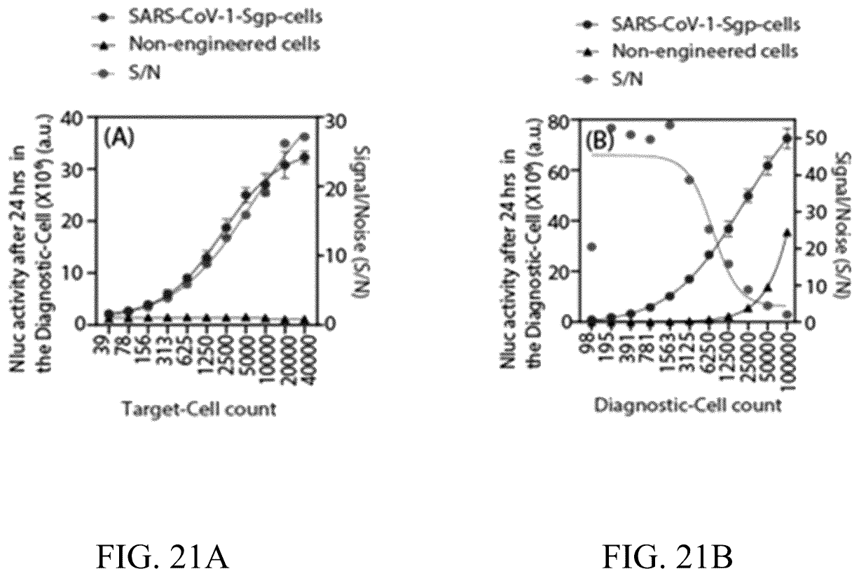

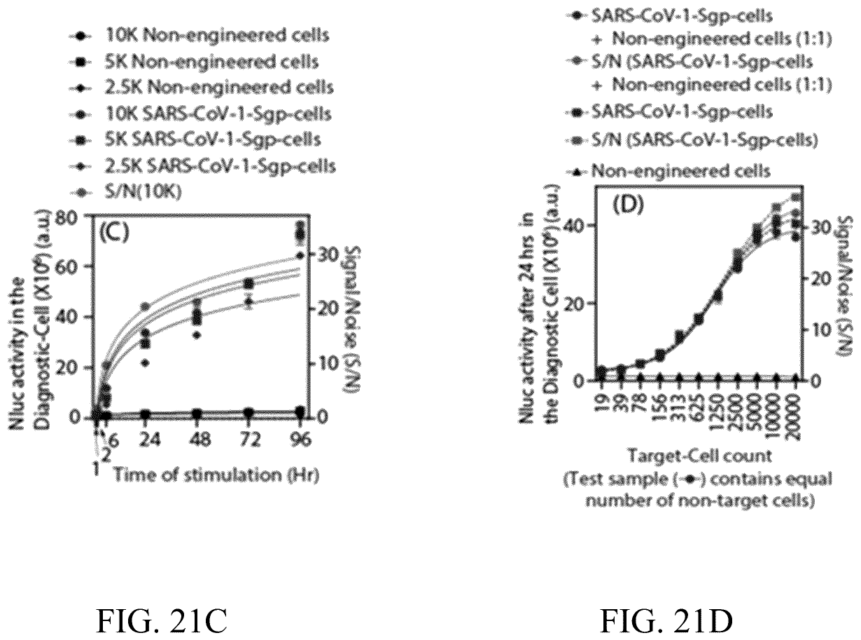

To demonstrate the functionality of the diagnostic cell in a biosafety level 2 (BSL2) laboratory, Sgp-expressing cells were engineered to simulate target host cells with a SARS-CoV-2 infection, sometimes referred to as “antigen-producing cells”. To demonstrate cell capability, the parental HEK293T/17 cell line was engineered to stably express the Sgp from SARS-CoV-2 (SARS-CoV-2-Sgp-cells). A non-engineered parental cell line was used as a negative control. The diagnostic cell was engineered to express GFP linked to Nluc through a self-cleavable 2A peptide linker (GFP-2A-Nluc) as target-inducible reporters for quantitative assessment of the infection. The binding domain of the diagnostic cell included the anti-Sgp VHH sequence (VHH-72; PDB: 6WAQ), which neutralizes zoonotic betacoronavirus infections (SARS-CoV-2 and SARS-CoV-1). The binding of Sgp on the target via the binding domain (see schematic in the FIG. 5B) results in the formation of immune synapse. The resulting activation of the diagnostic cell transcriptional machinery through intracellular calcium rise quantitatively informs on the infection burden by upregulation of the reporter proteins. The VHH portion can be replaced by the variable heavy-light (V_{H}-V_{L}) portion of the scFv of antibodies to provide specificity against any desired antigen. To further demonstrate the ability of the diagnostic cell to identify other SARS coronaviruses, a parallel validation of the VHH-72 diagnostic cell with target SARS-CoV-1-Sgp-cells (expressing the Sgp from SARS-CoV-1) was conducted. Data in FIGS. 21A-21D show that the SARS-CoV-1 antigen test exhibited a pattern similar to that observed with SARS-CoV-2 in FIGS. 18A-18D.

FIGS. 18A-18D illustrate plots characterizing activation of example genetically engineered diagnostic cells in response to antigen-presenting target cells, in accordance with the present disclosure. The Nluc activity in the VHH-72 diagnostic cell is proportional to the number of target SARS-CoV-2-Sgp-cells (as shown by FIG. 18A), proportional to the diagnostic cell count (target cells=2,500) (as shown by FIG. 18B), increased with respect to time and was significantly elevated within 1 hour (p<0.02; target cells=2,500) when stimulated by the target SARS-CoV-2-Sgp-cells versus non-target cells (as shown by FIG. 18C), and not affected in presence of non-target cells (as shown by FIG. 18D). Nluc activity for all observations was measured using n=4, error bars indicate ±1 SD and can also be considered as one half the width of a 68% confidence interval for the mean. VHH-72 diagnostic cell (12,500 cells) were used for FIGS. 18A, 18C, and 18D.

The results in FIG. 18A demonstrate that, in addition to the qualitative detection of the infection, the Nluc activity of diagnostic cell provides a quantitative measure that is proportional to the infection burden. The signal-to-noise ratio (S/N), as defined in the figure legend, quantifies the sensitivity of the diagnostic cell. The S/N was around 1.5 at a low infection burden (e.g., around 40 target cells or diagnostic cell to target cell ratio [D:T]=320:1, p<0.002) and exponentially increased to around 25 at a high infection burden (e.g., at 40,000 target cells [D:T=1:3.2; p<0.002]). This corroborates computational findings by others regarding the efficacy of cell-based immune targeting at lower D:T ratios.

FIG. 18B shows that a lower diagnostic cell count offers more sensitive detection when stimulated by the same number of targets. This finding is supported by the observation that the S/N is around 1.6 at 100,000 diagnostic cells (D:T=40:1; p<0.0001) and increases to around 40 at a lower diagnostic cell count (D:T=1:12.5; p<0.0001). Although this result comes at the expense of longer test durations, the data shows reduced non-specific interference when lower diagnostic cell counts are used. This is important in the context of developing a sensitive diagnostic test where a higher S/N ratio reduces false negatives and false positives.

FIG. 18C illustrates the Nluc reporter kinetics of the diagnostic cell when it is stimulated by serially diluted antigen-expressing cells or engineered target cells, e.g., engineered target SARS-CoV-2-Sgp-cells, and compared to the control cells. While the Nluc activity was already increased when compared to the control sample at 1 hour (for 2,500 SARS-CoV-2-Sgp-cells, p<0.02), it continued to increase for at least 96 hours. This finding can help in developing an antigen test for mass screening, as the wide readout window will accommodate a large number of patient specimens for assessing the disease penetration in the population. A representative S/N curve is included for 10,000 SARS-CoV-2-Sgp-cells, which increased with the duration of assay (e.g., for D:T of 0.8:1, S/N was around 1 at 1 hour [p=0.8] and at ˜17 at 96 hours [p<0.0001]). While the signal from the diagnostic cell was increased in correlation to a higher infection burden, as represented by the SARS-CoV-2-Sgp-cell count, the kinetics of the test was faster at a lower infection burden. For example, compared to the control cells, the signal was elevated at 1 hour, when 2,500 SARS-CoV-2-Sgp-cells (p<0.02, S/N around 1, D:T=5:1) were used, and at 6 hours, when 5,000 SARS-CoV-2-Sgp-cells (p<0.0001, S/N around 3.5, D:T=2.5:1) or 10,000 cells (p<0.0001, S/N around 5.5, D:T=1.2:1) were used. This also validates the previous observation that the Nluc activity of the diagnostic cell was proportional to the infection burden (FIG. 18A) and exhibited higher S/N at a limited engagement of the diagnostic cell with the targets (FIG. 18B).

To investigate the precision with which the samples should be prepared and any non-specific signal from the impurities, the genetically engineered Sgp-expressing target cells and non-engineered control cells in equal numbers were mixed (e.g., 1:1) and serially diluted the cell mixture to stimulate the diagnostic cell. Data in FIG. 18D show that, in comparison to stimulation by only engineered target cells, the loss in Nluc reporter activity of the diagnostic cell was insignificant (p>0.05 at all D:T). This confirms that there is no contribution of the background noise to the signal generated from the diagnostic cell. As observed in FIG. 18A, the Nluc reporter expression was again proportional to the target cell numbers. These findings demonstrate the sensitivity of the assay with a heightened reporter signal and provide the rationale for applying it to a large number of patient samples with minimal time for sample preparation and purification.

Various experiment were directed to developing and characterizing the diagnostic cell in a BSL2 containment facility using genetically engineered Sgp-expressing target cells including SARS-CoV-2-Sgp-cells (FIGS. 18A-18D) and SARS-CoV-1-Sgp-cells (FIGS. 21A-21D).

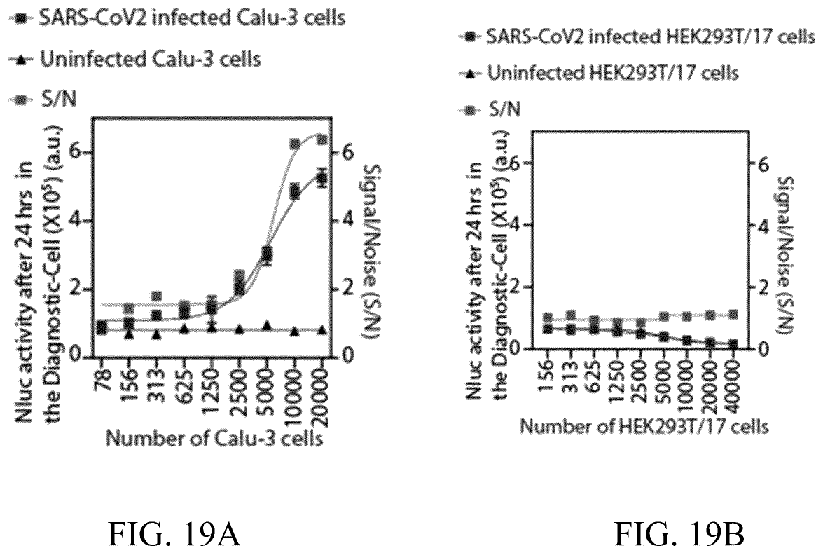

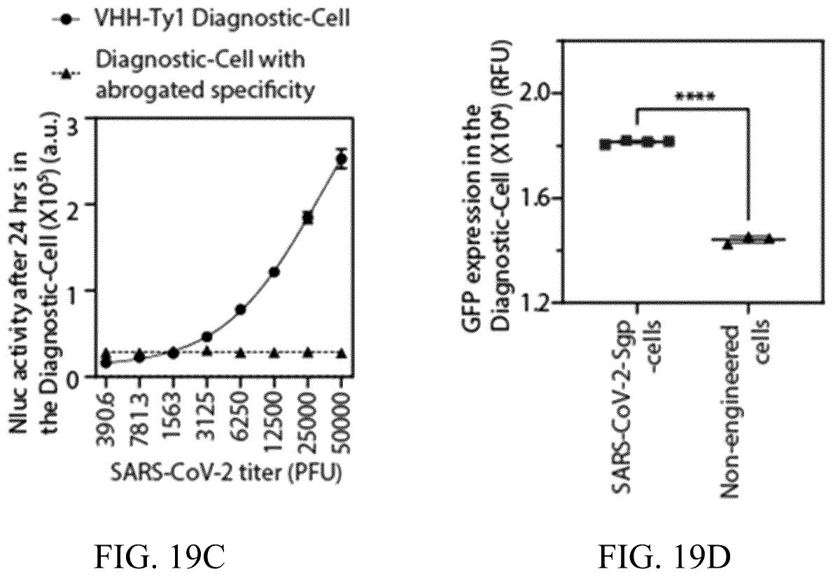

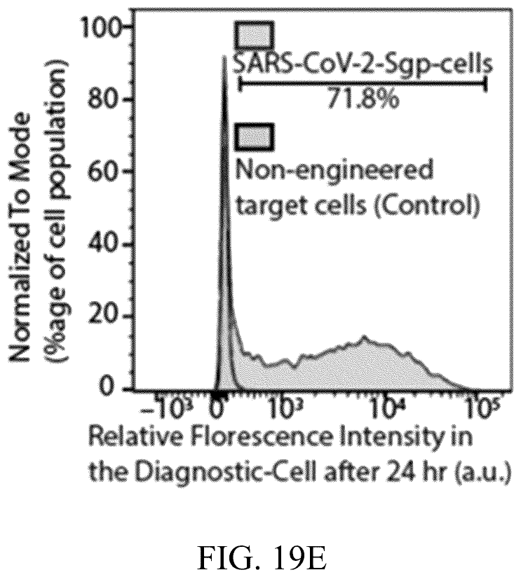

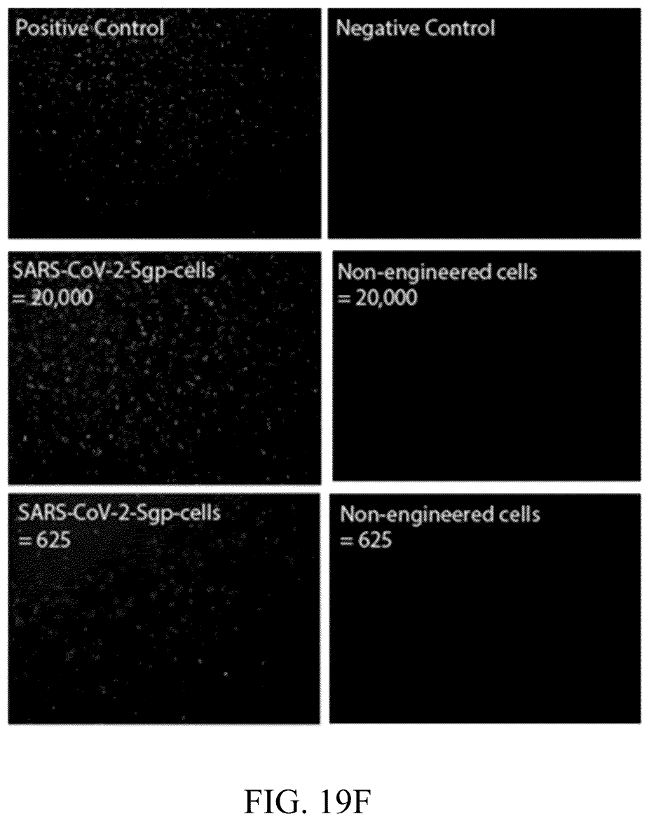

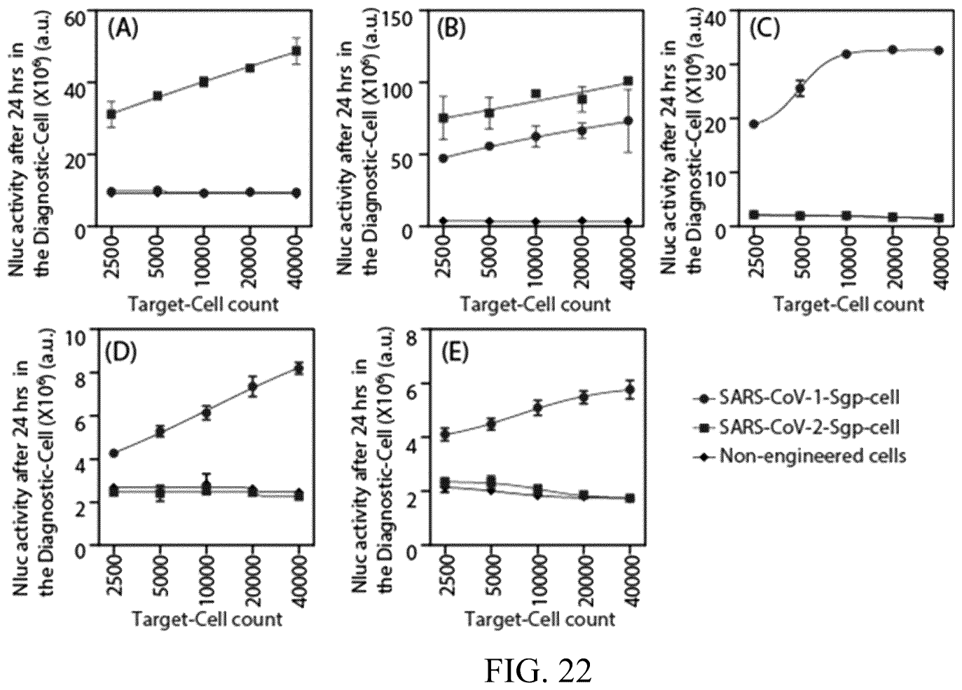

FIGS. 19A-19F show example data results of implementing an example diagnostic cell with infectious SARS-CoV-2 virus particles (Isolate: Hong Kong/VM20001061/2020) in a BSL3 containment facility, in accordance with the present disclosure. Toward this goal and to impart specificity to the diagnostic cell against the ongoing pandemic, the VHH-72 Sensor domain of diagnostic cell was exchanged with the VHH portion of another antibody (VHH-Ty1; PDB: 6ZXN) that is specific to the Sgp of SARS-CoV-2. The rationale for selecting VHH-Ty l was that it can potentially reduce false positives because it does not cross-react with SARS-CoV-1 (see FIG. 22 for validation studies) or other seasonal human coronavirus strains (HKU1, OC43, 229E, and NL63). FIG. 22 describes experiments to identify the binding domain among multiple candidates for not cross-reacting with SARS-CoV-1. Initial investigations were conducted by assessing the target-inducible Nluc activity in the VHH-Tyl diagnostic cell and were later validated by assessing the GFP signal. Negative control was composed of uninfected host cells or a diagnostic cell with abrogated specificity.

More particular, FIGS. 19A-19F show results from assessing a VHH-Tyl diagnostic cell activation by infected host cells, SARS-CoV-2 virus or genetically engineered target SARS-CoV-2-Sgp-cells. FIG. 19A shows that the Nluc activity in the VHH-Tyl diagnostic cell is proportional to the number of SARS-CoV-2-infected Calu-3 target cells (n=3). FIG. 19B shows that no Nluc activity was observed in the VHH-Tyl diagnostic cell when SARS-CoV-2 treated HEK293T/17 cells (lacks receptors for SARS-CoV-2) were used as targets. FIG. 19C shows that the Nluc activity in the VHH-Ty 1 diagnostic cell increased with respect to the number of viral particles present in solution. FIG. 19D shows that GFP expression in the VHH-Tyl diagnostic cell was assessed using plate reader (target cells=2,500). FIG. 19E shows that GFP expression in the VHH-Tyl diagnostic cell was assessed using flow-cytometry. FIG. 19 F is a microscope image panel showing GFP expression in the VHH-Tyl diagnostic cell at different D:T ratios. All images taken at 4× magnification. Positive control was prepared by chemically stimulating the diagnostic cell. Unstimulated diagnostic cell was used as negative control. Statistical significance was calculated using the two-tailed student's t-test test. Nluc activity for all observations was measured using n=4, error bars indicate ±1 SD. For FIGS. 19A, 19B, 19D, and 19F, VHH-Ty l diagnostic cell=12,500 cells; for FIG. 19C VHH-Ty 1 diagnostic cell=20,000.

FIG. 19A demonstrates the quantitative Nluc reporter response of the VHH-Ty1 diagnostic cell. The Nluc activity on engaging Calu-3 host cells infected with competent SARS-CoV-2 virus exhibited a trend similar to that observed with engineered SARS-CoV-2-Sgp-cells in FIG. 18A and was proportional to the infection burden. At a low infection burden, the S/N was around 1 (p=0.83; D:T ˜160:1), which exponentially increased to around 6 at a high infection burden at 20,000 target cells (p<0.0001; D:T=1:1.6). FIG. 19B shows a similar assay conducted by replacing the Calu-3 cells with HEK293 cells. Lack of Nluc expression in the VHH-Ty 1 diagnostic cell, when stimulated by HEK293T/17 cells treated with infectious SARS-CoV-2 virus correlates with lack of SARS-CoV-2 tropism for targeting HEK293T/17 cells that may not express receptors for virus attachment.

To develop an effective antigen test indicative of active infection, experiments were directed to confirming the diagnostic cell can detect virus particles. FIG. 19C shows the exponential increase of Nluc activity in the diagnostic cell after engaging serially diluted SARS-CoV-2 virion particles ranging from 112,500 PFU to 880 PFU (Nluc activity with respect to the viral titer). This finding is unique, as the lack of co-stimulatory molecules on virus particles, which are present on the mammalian host cell and are essential for stimulating the T-cells, challenges the possibility of diagnostic cell successfully detecting target virus particles. The fact that the diagnostic cell can detect virus particles may be explained by the cross-linking of multiple Sgp-specific VHH on the diagnostic cell surface assisted by the Sgp of SARS-CoV-2 particles. This initiates the intracellular activation-signaling cascade. Findings in FIG. 19C validate that it is, in fact, possible for the virions to directly activate the antigen-specific diagnostic cell.

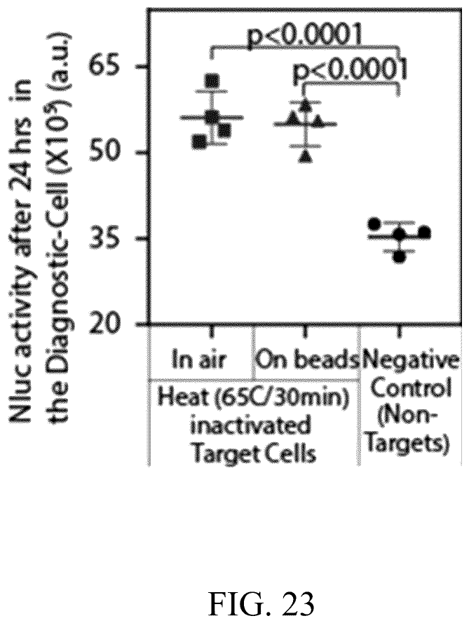

To further reduce the operational cost of the diagnostic cell, the potential of target-inducible GFP expression in the diagnostic cell was explored to assess the status of active infection. The results of experiments performed using the engineered SARS-CoV-2-Sgp-cells (FIGS. 19D-19F) affirm GFP-based sensing of target cells using the VHH-Tyl diagnostic cell. This finding reduces the reliance of diagnostic cell on a bioluminescent reporter (Nluc), which requires an additional step of substrate addition and contributes toward the cost of the test. To eliminate chances of infection during the handling of infected specimens, the use of diagnostic cell was validated with samples that underwent heat-inactivation (65° C. for 30 minutes). The Nluc signal from diagnostic cell is significantly elevated (p<0.0001, FIG. 23) and differentiates between engineered Sgp-expressing cells and non-engineered cells.

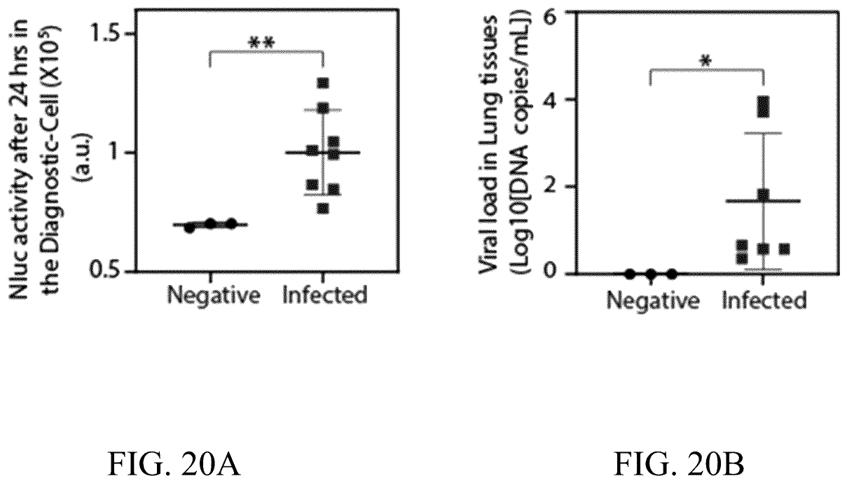

FIGS. 20A-20B illustrate results of detecting infection in mouse oropharyngeal swabs using an example diagnostic cell, in accordance with the present disclosure. The VHH-Tyl diagnostic cell was further assessed to detect active infection in specimens obtained from both infected and healthy mice via oropharyngeal swabs. Results validate that the diagnostic cell can diagnose active COVID-19 infection via oropharyngeal swabs (FIG. 20A). For the proof-of-principle studies, the signal from the diagnostic cell after 24 hours was assessed. The results in FIG. 18C show that the test can be significantly faster with the potential to inform on the active infection within 1 hour (for 2,500 SARS-CoV-2-Sgp-cells). FIG. 20B shows results obtained by qPCR (as the gold standard) while using mouse lung tissues for comparison with the results from diagnostic cell. These results demonstrate that the diagnostic cell can be rapidly deployed at low cost toward emerging viruses and be used with oral specimens without any sample preparation or signal-amplification step.

FIG. 20A is a scatter plot that shows that the VHH-Tyl diagnostic cell differentiates between infected and negative throat swab samples (p<0.05). (Infected mice, n=8 and Negative mice, n=3). Each data point represents average Nluc readings from 2 swabs. FIG. 20B is scatter plot that shows qPCR results for the lung tissues collected from the same mice (lung tissue for one infected mouse was not collected). Statistical significance was calculated using the two-tailed student's t-test with Welch's correction, error bars indicate ±1 SD.

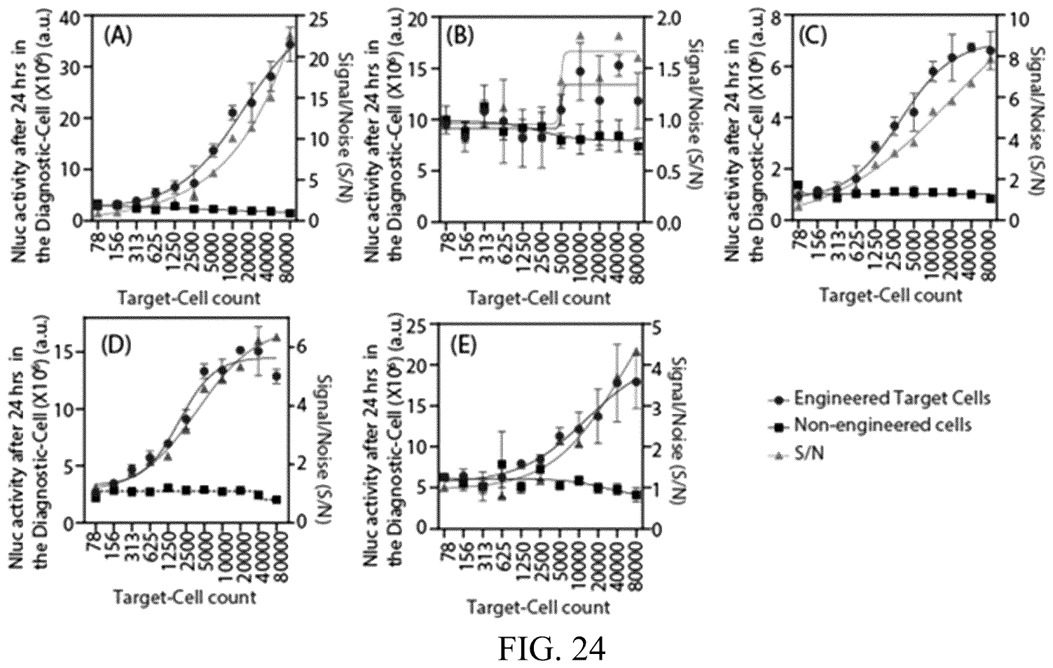

The impact of this technology goes beyond the current COVID-19 pandemic. To show the expanded applicability of the diagnostic cell, multiple different diagnostic cells were generated to detect multiple viruses with the potential to cause the future pandemics. FIG. 24 shows the feasibility of the Diagnostic-Cell technology against Ebola, Marburg, West Nile Virus, Chikungunya, and Nipah using engineered Target Cells to present virus-specific envelope proteins. A fully matured approach will eliminate the long development periods needed to apply a separate, specific approach for each virus or its variants, which is currently the case for SARS-CoV-2, and rapidly deliver a diagnostic test for any pathogen in less than a month.

The experiments also confirmed the rapid scalability of the diagnostic cell for performing diagnostic antigen test. The doubling time of the VHH-Ty 1 diagnostic cell was experimentally determined to be 24.3 (+6.8) hours. Further calculations show that with 10 million diagnostic cell, it is possible to scale up to meet the target for 30 million tests/day in less than 20 days.

FIGS. 21A-21D illustrate example results of assessing VHH-72 diagnostic cell activation by genetically engineered target SARS-CoV-1-Sgp-cells, in accordance with the present disclosure. As shown by FIG. 21A, the Nluc activity in the VHH-72 diagnostic cell is proportional to the number of target SARS-CoV-2-Sgp-cells. As shown by FIG. 21B, the Nluc activity in the VHH-72 diagnostic cell is proportional to the diagnostic cell count (target cells=2,500). As shown by FIG. 21C, the Nluc activity in the VHH-72 diagnostic cell increased with respect to time when stimulated by the target SARS-CoV-1-Sgp-cells versus non-target cells (target cells=2,500). As shown by FIG. 21D, the Nluc activity in the VHH-72 diagnostic cell is not affected in presence of non-target cells. Nluc activity for all observations was measured using n=4, error bars indicate ±1 SD and can also be considered as one half the width of an 68% confidence interval for that mean. VHH-72 diagnostic cell (12,500 cells) was used for FIGS. 21A, 21C, and 21D.

FIG. 22 illustrates example results of screening of different diagnostic cells with specificity towards SARS-CoV-2 and SARS-CoV-1, in accordance with the present disclosure. The Nluc activity in different diagnostic cell types is proportional to the number of target cells (SARS-CoV-2-Sgp-cells or SARS-CoV-1-Sgp-cells). The binding domain sequences were obtained from (A) VHH-Tyl, (B) S309 antibody, (C) S230 antibody, (D) m396 antibody, and (E) CR3022 antibody. In all experiments, diagnostic cell=12,500 and Nluc activity for all observations was measured using n=4; error bars indicate ±1 SD and can also be considered as one half the width of an 68% confidence interval for that mean.

FIG. 23 illustrates detection of heat-inactivated target cells by example VHH-Tyl diagnostic cells, in accordance with the present disclosure. A scatter plot shows the Nluc activity in the VHH-Tyl diagnostic cells (12,500 cells) upon interacting with engineered Sgp-expressing target cells (SARS-CoV-2-Sgp-cells=5,000 cells) heat-inactivated in air or on beads at 65° C. vs. non-engineered cells. Nluc activity for all observations was measured using n=4; error bars indicate ±1 SD.

FIG. 24 illustrates example assessment of different diagnostic cells generated with specificity to other emerging viruses, in accordance with the present disclosure. The Nluc activity the different types of diagnostic cells is specific to their targets and is proportional to the number of target cells engineered for surface expression with envelop proteins. The sequences for the envelop proteins were obtained from (A) Ebola virus; (B) Marburg virus; (C) Chikungunya virus; (D) Nipah virus; and (E) West-Nile virus. In all experiments, diagnostic cells=12,500 and Nluc activity for all observations was measured using n=4; error bars indicate ±1 SD and can also be considered as one half the width of a 68% confidence interval for that mean

Table 2 below provides difference sequences used to generate the diagnostic cells.

TABLE 2

| Genetic Element | SEQ ID NO |
|---|---|
| SARS-CoV-2 Sgp | SEQ ID NO: 1 |
| SARS-CoV-1 Sgp | SEQ ID NO: 2 |
| VHH-72 antibody | SEQ ID NO: 3 |
| VHH-Ty1 antibody | SEQ ID NO: 25 |
| S309 antibody | SEQ ID NO: 26 |
| S230 antibody | SEQ ID NO: 27 |
| m396 antibody | SEQ ID NO: 28 |
| CR3022 antibody | SEQ ID NO: 29 |
| Ebola virus Egp | SEQ ID NO: 30 |
| Ebola virus Egp antibody | SEQ ID NO: 31 |
| Marburg virus Egp | SEQ ID NO: 32 |
| Marburg virus Egp antibody | SEQ ID NO: 33 |
| Chikungunya virus Egp | SEQ ID NO: 34 |
| Chikungunya virus Egp antibody | SEQ ID NO: 35 |
| Nipah virus Egp | SEQ ID NO: 36 |
| Nipah virus Egp antibody | SEQ ID NO: 37 |
| West-Nile virus Egp | SEQ ID NO: 38 |
| West-Nile virus Egp antibody | SEQ ID NO: 39 |
| Luc2 | SEQ ID NO: 8 |
| E2 Crimson | SEQ ID NO: 9 |
| Luc2-P2a-E2 Crimson | SEQ ID NO: 10 |
| GFP | SEQ ID NO: 11 |
| Nluc | SEQ ID NO: 12 |
| P2a | SEQ ID NO: 13 |
| GFP-P2a-Nluc | SEQ ID NO: 14 |
| Plasmid 2 (SARS-CoV-2 Egp) | SEQ ID NO: 16 |
| Plasmid 3 (SARS-CoV-1 Egp) | SEQ ID NO: 17 |
| Plasmid 4 (VHH-antibody) | SEQ ID NO: 18 |
| Plasmid 11 (VHH-Ty1 antibody) | SEQ ID NO: 40 |
| Plasmid 12 (S309 antibody) | SEQ ID NO: 41 |
| Plasmid 13 (S230 antibody) | SEQ ID NO: 42 |
| Plasmid 14 (m396 antibody) | SEQ ID NO: 43 |
| Plasmid 15 (CR2033 antibody) | SEQ ID NO: 44 |
| Plasmid 16 (Ebola virus Egp) | SEQ ID NO: 45 |
| Plasmid 17 (Ebola virus Egp | SEQ ID NO: 46 |
| antibody) | |
| Plasmid 18 (Marburg virus Egp) | SEQ ID NO: 47 |
| Plasmid 19 (Marburg virus Egp | SEQ ID NO: 48 |
| antibody) | |
| Plasmid 20 (Chikungunya virus | SEQ ID NO: 49 |
| Egp) | |
| Plasmid 21 (Chikungunya virus | SEQ ID NO: 50 |
| Egp antibody) | |
| Plasmid 22 (Nipah virus Egp) | SEQ ID NO: 51 |
| Plasmid 23 (Nipah virus Egp | SEQ ID NO: 52 |
| antibody) | |
| Plasmid 24 (West Nile virus Egp) | SEQ ID NO: 53 |
| Plasmid 25 (West Nile virus Egp | SEQ ID NO: 54 |
| antibody) | |

Synthesis and Experimental Information for Diagnostic Cell

(1) Materials and reagents. Engineered Jurkat E6-1 (ATCC, Cat #TIB-152) cell lines were maintained in complete RPMI media [RPM11640 (Corning, Cat #10-040-CV), 1000 were heat-inactivated fetal bovine serum (FBS) (Sigma-Aldrich, Cat #F2442-500ML) and 1× Penicillin-Streptomycin solution (Corning, Cat #30-002-Cl)]. Parental and Engineered HEK293T/17 cells (ATCC, Cat #CRL-11268) were cultured in complete DMEM [DMEM growth media (Corning, Cat #10-013-CV) supplemented with 10% FBS (Sigma-Aldrich, Cat #F2442-500ML) and 1× Penicillin-Streptomycin solution (Corning, Cat #30-002-Cl)]. Calu-3 cells (ATCC Cat #HTB-55) were cultured in complete EMEM [EMEM growth media (Corning, Cat #10-009-CV) supplemented with 10% heat-inactivated fetal bovine serum (FBS) (Sigma-Aldrich, Cat #F2442-500ML) and 1× Penicillin-Streptomycin solution (Corning, Cat #30-002-Cl)]. All cells were expanded, and liquid nitrogen stocks were maintained using freezing media (50% FBS, 40% growth media and 10% DMSO). The SARS-CoV-2 virus culture [BEI Resources, NIH; Hong Kong/VM20001061/2020 (Cat #NR-52282)] was provided by SRI International. Plasmids encoding different genetic payloads (transfer plasmids) were designed in SnapGene software (GSL Biotech LLC) and sub-cloned into lentivirus vector plasmid (System Biosciences, Cat #CD510B-1) of piggyBac Transposon vector plasmid (System Biosciences, Cat #PB510B-1). Plasmids encoding 2^{nd} generation packaging genes (psPAX2—Cat #12260, pMD2.G—Cat #12259) were received from Didier Trono (Ecole Polytechnique Federale de Lausanne, Lausanne, Switzerland) through Addgene, while pAdvantage was obtained from Promega (Cat #E1711). piggyBac Transposase sequence was provided by Johns Hopkins University School of Medicine. All plasmid preparation services (chemical synthesis of DNA insert sequences, sub-cloning into respective vector backbones, and the amplification) were obtained from Epoch Life Science, Inc. (Missouri City, TX). Transporter 5™ reagent (Polysciences, Inc, Cat #26008-5) was used to transfect parental HEK293T/17 cells during lentivirus production. The collected lentivirus was transduced into Jurkat cells using Polybrene (Abm®, Cat #G062). TransIT®-2020 transfection reagent (Mirus #MIR5400) was used to transfect piggyBac Transposon system plasmids into parental HEK293T/17 cells to engineer stable antigen-presenting cells (APCs) or Sgp-expressing target cells. Puromycin dihydrochloride (ThermoFisher Scientific, Cat #A1113803) was used for selecting stable cells. Nano-Glo® assay (Promega, Cat #N1120) was used to assess the NanoLuc® (Nluc) expression activity. Sterile mini-tip polyester swabs (Puritan, Cat #25-800-1PD) were used for collection of oropharyngeal swab samples from mice. For RNA preparations from lung tissues, Direct-zol RNA Mini-Prep Kit (Zymo Research, Cat #11-331) and reverse transcription by SuperScript III RT (Invitrogen, Cat #18080044) were used following the manufacturers' instructions. TaqMan Universal PCR Master Mix (Applied Biosystems, Cat #4305719) was used for RT-qPCR reactions.

(2) Lentivirus production. Lentivirus particles were prepared by packaging the transfer plasmid using 2^{nd} generation lentivirus system.

(3) Engineering of diagnostic cells. The Jurkat E6-1 suspension cell line was engineered with lentivirus particles carrying the genetic payload (FIG. 5B) with the artificial cell signaling pathway. The binding domain sequences were selected based on their specificities to the envelop proteins of the respective viruses. The cells were treated with lentivirus in the presence of 8 μg/mL Polybrene. After 48 hours, the engineered cells were placed in selection using 0.5 μg/mL of puromycin dihydrochloride. The unmodified parental cell line was also placed under selection as a positive control for cell killing by puromycin. Following selection, cells were expanded as required for different assays and frozen using freezing media. Sequences from the binding domain were obtained from the following sources: VHH-72 (PDB: 6WAQ), VHH-Tyl (PDB: 6ZXN), S309 antibody (PDB: 6WPS), S230 antibody (PDB: 6NB8), m396 antibody (PDB: 2DD8), CR3022 antibody (PDB: 6W41), Ebola virus antibody (PDB: 6MAM), Marburg virus antibody (PDB: 5JRP), Chikungunya virus antibody (PDB: 4GQ9), Nipah virus antibody (PDB: 6U1T), and West-Nile virus antibody (PDB: 1ZTX). All gene sequences were codon optimized before expression using lentivirus plasmids.

(4) Engineering of target cells with surface expression of virus envelop protein. The parental HEK293T/17cells were engineered to stably express the virus envelop protein, using the piggyBac Transposon system, as previously described. Two plasmids were designed with the piggyBac transposon vector backbone for cell surface expression of the envelop protein from the respective virus. A monolayer of HEK293T/17 cells were transfected with the transposon plasmid (carrying the gene of interest) and transposase plasmid, in a ratio of 2.5:1, respectively, using TransIT®-2020 transfection reagent. After 48 hours of transfection, the transfected cells were placed under selection using 0.5 μg/mL of Puromycin dihydrochloride. The unmodified parental HEK293T/17 cell line was placed under selection as a positive control for cell killing by the antibiotics. The generated stable cell lines were expanded as required for different assays. Sequences from the gene of interest (envelop proteins) were obtained from the following sources: SARS-CoV-2 (GenBank: QHD43416.1, position 1-1273), SARS-CoV-1 (GenBank: AAP13567.1, position 1-1255), Ebola virus (GenBank: AAB81004.1, position 33-676), Marburg virus (GenBank: CAA78117.1, position 19-681), Chikungunya (E1E2) virus (GenBank: AGX45493.1, 339-1247), Nipah virus Fusion gp (GenBank: Q9IH63.1, position 27-546), and West-Nile virus (GenBank: AAT11537.1, position 1-501). All gene sequences were codon optimized before expression in the piggyBac Transposon system.

(5) Method of use for the diagnostic cell [D] with genetically engineered target cells [T]. The diagnostic cell and target cells were co-cultured at different diagnostic cell to target cell (D:T) ratios in 100 μL of complete RPMI media in a single well of a 96-well plate. Phorbol myristate acetate (30 nM) and ionomycin (1 μM) solution mixed in 0.5% Dimethyl sulfoxide (DMSO) was used for unspecific stimulation of the diagnostic cell (positive control). Negative control comprised of stimulation by 0.5% DMSO. After the specified time in co-culture, NanoLuc® (Nluc) activity in the diagnostic cell was assessed using the Nano-Glo® assay following the manufacturer's instructions. The Nluc enzyme substrate was diluted in the cell lysis buffer provided with Nano-Glo® and added to the co-cultures in 96-well plate for assessing enzyme activity. Following a brief incubation period of 3 minutes, bioluminescence was read on a microplate reader (Perkin Elmer, EnVision™ Multilabel Plate Reader Model: 2104-0010A). Additionally, GFP expression in the diagnostic cell was assessed using the plate-reader, flow-cytometry (FACS Ariam III, BD Biosciences), and fluorescence microscopy (EVOS FL Auto 2, Invitrogen).

(6) Method of use for the VHH-Tyl diagnostic cell [D] with SARS-CoV-2-infected Calu-3 or HEK293T/17 epithelial cells as infected target cells [T]. Human lung epithelial cells, Calu-3 cells (or HEK293T/17 cells) were cultured in a 6-well plate (1×10⁶/well) overnight in complete EMEM media and infected as previously described. At about 70% confluency, the cells were infected with the SARS-CoV-2 virus culture at an MOI of 0.05 for 24 hours. The infected cells were harvested and then used as the antigen-presenting target cells in co-culture experiments. The virus-infected target cells were co-cultured at different D:T with the VHH-Tyl diagnostic cell in 100 μL of complete RPMI media in a single well of a 96-well plate. Non-infected cells were used as the negative controls. After 24 hours of co-culture, Nluc activity in the VHH-Ty l diagnostic cell was assessed using the Nano-Glo® assay.

(7) Method of use for the VHH-Tyl diagnostic cell with SARS-CoV-2 viral particles. To test if the VHH-Tyl diagnostic cell could be used to detect presence of SARS-CoV-2 viral particles in solution, the diagnostic cell was cultured in 100 μL per well of complete RPMI media containing varying concentrations of serially diluted SARS-CoV-2 viral particles in a 96-well plate (20,000/well). Another diagnostic cell with an abrogated sensor specificity was used as a negative control. The plate was incubated at 37° C. for 24 hours before Nluc activity was assessed using Nano-Glo assay.

(8) Method of use for the VHH-Ty l diagnostic cell to detect SARS-CoV-2 infections in animal samples. To check if the diagnostic cell can detect active infection in animal samples, we used a mouse model. Eight (8) heterozygous female K18-hACE c57BL/6J mice (strain: 2B6.Cg-Tg(K18-ACE2) 2Prlmn/J, The Jackson Laboratory) were infected intranasally using 50 μL of virus culture (5,000 PFU) per animal, following protocols approved by the Institutional Animal Care and Use Committee at SRI International (#20003), while three (3) mice were not infected (negative controls). Eight days post infection (DPI-8), the mice were euthanized and two oropharyngeal swab samples were collected from each animal. The swabs were immediately placed into collection tubes containing 500 μL of complete RPMI media and transported to the BSL3 lab for processing. For each collected swab, the collection tube was vortexed for 10-15 seconds to release cells into the media before the swab was removed from the collection tubes. The collection tube was then centrifuged at 300 g for 5 minutes to collect cell pellets. The cell pellet was then resuspended into 50 μL of complete RPMI media. In a 96-well plate, each sample (cells in 50 μL) was co-cultured with 50 μL of 25,000 VHH-Tyl diagnostic cell in complete RPMI media. The plate was incubated at 37° C. for 24 hours before Nluc activity was assessed using the Nano-Glo assay.

(9) Viral RNA extractions and RT-qPCR analysis. Total RNA was extracted from mouse lung tissue homogenates using the Direct-zol RNA MiniPrep Kit and reverse transcription was performed using SuperScript III RT, following the manufacturer's instructions. RT-qPCR reactions were performed using TaqMan Universal PCR Master Mix, in which samples were processed using the following cycling protocol in the ViiA 7 Thermocycler (Applied Biosystems): 50° C. for 2 minutes and 95° C. for 10 minutes, followed by 40 cycles at 95° C. for 15 seconds and 60° C. for 1 minute. The primer sequences used for RT-qPCR targeted the Nucleocapsid (NC) gene of SARS-CoV-2 and are as follows: Forward: 5′-GTTTGGTGGACCCTCAGATT-3′, Reverse: 5′-GGTGAACCAAGACGCAGTAT-3′ and Probe: 5′-/56-FAM/TAACCAGAA/ZEN/TGGAGAACGCAGTGGG/3IABkFQ/-3′. Assay validation was performed using SARS-CoV-2 virus genome to create a standard curve, and the detection limit was determined to be from 5×10⁶ to 0.5 viral RNA copies per mL. Results were expressed as log₁₀(viral RNA copies/mL).

(10) Statistical analysis. The experimental design for each panel in the figures is described below. GraphPad Prism 9.2.0 (GraphPad Software, Inc) was used to conduct all statistical analyses.

(i) FIGS. 18A-18D (VHH-72 diagnostic cell activation by engineered target SARS-CoV-2-Sgp-cells). Statistical analysis for all panels (FIGS. 18A-18D) was based on an unpaired two-tailed student's t-test with common variance, and the p-value of <0.05 was considered statistically significant. Comparison of data points was done by the FDR multiple comparison approach, using the two-stage step-up procedure of Benjamini, Krieger, and Yekutieli with FDR=1%, and only p-values less than an FDR of 1% were reported. The S/N is calculated as the ratio of the mean Nluc activity in the VHH-72 diagnostic cell when stimulated by the target cells (SARS-CoV-2-Sgp-cells) divided by the mean Nluc activity when stimulated by the negative controls (non-engineered parental HEK293T/17 cells). The error bars extend 1 SD above and below the mean and can also be considered as one half-width of a 68% confidence interval for the mean.

(ii) FIG. 18A (Nluc activity in diagnostic cell is proportional to the number of target cells). The Nluc activity in the diagnostic cell stimulated by the target (SARS-CoV-2-Sgp-cells) or non-target cells was fitted using a four-parameter logistic model for Nluc given by Nluc(X)=Nlucₘᵢₙ+{Nlucₘₐₓ−Nlucₘᵢₙ}/{1+10\hat{ }[b*(log₁₀[X*]−log₁₀[X])]}; where Nluc(X) is the value of Nluc at X, X is the Target-Cell count, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and X₅₀ is an estimated parameter representing the X value corresponding to (Nlucₘₐₓ−Nlucₘᵢₙ)/2.

(iii) FIG. 18B (Nluc activity increases with respect to amount of VHH-72 diagnostic cell). The Nluc activity in the diagnostic cell stimulated by the target (SARS-CoV-2-Sgp-cells) or non-target cells was fitted using a four-parameter logistic model Nluc(X) where X is the diagnostic cell count.

(iv) FIG. 18C (Nluc activity in diagnostic cell is a function of duration of stimulation). The Nluc activity in the diagnostic cell stimulated by the target (SARS-CoV-2-Sgp-cells) or non-engineered cells was fitted using the equation Y=a+b*log₁₀(X), where X is the stimulation time in hours.

(v) FIG. 18D (Sensitivity of diagnostic cell is unaffected by the presence of non-target cells). The Nluc activity in the diagnostic cell stimulated by the target (SARS-CoV-2-Sgp-cells) or non-target cells was fitted using a four-parameter logistic model Nluc(X) where X is the target cell count.

(vi) FIGS. 19A-19F (VHH-Ty1 diagnostic cell activation by infected host cells, SARS-CoV-2 virus or engineered target SARS-CoV-2-Sgp-cells). Statistical analysis for all panels as based on an unpaired two-tailed student's t-test with common variance and the p-value of <0.05 was considered statistically significant. Comparison of data points was done by the FDR multiple-comparison approach, using the two-stage step-up procedure of Benjamini, Krieger, and Yekutieli with FDR=1%, and only p-values less than an FDR of 1% were reported. The S/N is calculated as the ratio of the mean Nluc activity in the diagnostic cell when stimulated by the targets (infected Calu-3 or infected HEK293T/17 cells) divided by the mean Nluc activity when stimulated by the non-infected parental Calu-3 or HEK293T/17 cells. The error bars extend 1 SD above and below the mean and can also be considered as one half-width of a 68% confidence interval for the mean.

(vii) FIG. 19A and FIG. 19B (Nluc activity in diagnostic cell is proportional to the number of SARS-CoV-2-infected epithelial cells). The Nluc activity in the diagnostic cell stimulated by the targets (infected Calu-3 or infected HEK293T/17 cells) or non-targets (non-infected Calu-3 or non-infected HEK293T/17 cells) was fitted using a four-parameter logistic model Nluc(X) where X is the infected target cell count.

(viii) FIG. 19C (Nluc activity in diagnostic cell is proportional to the number of SARS-CoV-2 viral particles in solution). The Nluc activity in the VHH-Ty 1 diagnostic cell (or diagnostic cell with an abrogated sensor specificity used as a negative control) stimulated by the SARS-CoV-2 viral particles was fitted using a four-parameter logistic model Nluc(X) where X is the viral particle count (PFU).

(ix) FIG. 19D (Detection of GFP expression in the VHH-Ty 1 diagnostic cell using a plate-reader). The scatter plot shows the Nluc activity when the diagnostic cell was stimulated by SARS-CoV-2-Sgp-cells or parental HEK293T/17 cells. An unpaired student's two-tailed t-test was used to determine the statistical difference between the two samples, assuming a common variance and a p-value of 0.05.

(x) FIG. 19E (Detection of GFP expression in the VHH-Ty 1 diagnostic cell 1 using Flow-cytometry). Expression of GFP (Relative Fluorescence Intensity) in the diagnostic cell is analyzed using Flow-Jo software 10.8.0 (BD Biosciences) and the cell counts are normalized to the respective cell type.

(xi) FIG. 20A (Detection of SARS-CoV-2 infection in mouse oropharyngeal swabs using the VHH-Ty l diagnostic cell). A scatter plot (confidence interval of 95%) shows the difference between throat swabs collected from SAR-CoV-2-infected mice and non-infected mice, using an unpaired two-tailed student's t-test with Welch's correction and assuming Gaussian distribution.

(xii) FIG. 20B (Diagnosis of SARS-CoV-2 infection in mouse lung tissues by qPCR). A scatter plot (confidence interval of 95%) shows the difference between viral loads of mouse lungs tissues collected from SAR-CoV-2-infected mice and non-infected mice, using an unpaired two-tailed students' t-test with Welch's correction and assuming Gaussian distribution.

(xiii) FIGS. 21A-21B (VHH-72 diagnostic cell activation by engineered target SARS-CoV-1-Sgp-cells). Statistical analyses for SARS-CoV-1 related investigations reported in all panels (A-D) were similar to that employed for SARS-CoV-2 in the respective panels of FIG. 18A-18D.

(xiv) FIG. 22 (Screening of different diagnostic cells with specificity towards SARS-CoV-2 and SARS-CoV-1). The Nluc activity in the diagnostic cell stimulated by the target (SARS-CoV-2-Sgp-cells or SARS-CoV-1-Sgp-cells) or non-target cells was fitted using a four-parameter logistic model Nluc(X) where X is the target cell count.

(xv) FIG. 23 (Detection of heat-inactivated target cells by the VHH-Ty 1 diagnostic cell). A scatter plot (confidence interval of 95%) shows the difference in Nluc expression signal in the diagnostic cell by heat-inactivated engineered Sgp-presenting target cells (by air or beads at 65° C.) versus non-engineered cells, using an unpaired two-tailed students' t-test, assuming Gaussian distribution and that both sample means have the same standard deviations.

(xvi) FIG. 24 (Development of different diagnostic cells with specificity to other emerging viruses). The Nluc activity in the diagnostic cell stimulated by different target cell types (engineered target cells displaying the glycoproteins from (A) Ebola virus; (B) Marburg virus; (C) Chikungunya virus; (D) Nipah virus; and (E) West-Nile virus) or non-target (non-engineered parental HEK293T/17) control cells was fitted using a four-parameter logistic model Nluc(X), where X is the target cell count.

Other experimental embodiments were directed to transforming T-cells to generated genetically modified reporter cells, which can be used to provide qualitative immunity information and/or assess immunity of a population via serology tests. For example, a sampling of hosts (e.g., subjects) of a population can be assessed to provide population status information on protective immunity, disease prevalence, and efficacy of vaccination programs. Such data can be gathered using serology tests that are scalable and have high-throughput with rapid turn-around time. In various experimental embodiments, a serology test platform was generated and assessed that used a pair of genetically engineered cells (e.g., reporter cell and test cell) to rapidly detect IgG antibodies with specificity for SARS-CoV-2. The serology test is a low-cost option for screening mass populations and has the potential for rapid scale-up. The serology test can be used to screen individuals for the presence of antibodies and measure their anti-viral immunity.

Various experimental embodiments were directed to generating and assessing the serology test platform, an antibody-specific reporter cell complex that can be used to identify individuals harboring antibodies against specific viral antigens. The test platform was validated by detecting antibodies against SARS-CoV-2 from clinical specimens. This platform can be quickly redirected to identify antibodies against any virus by generating different target cells. This technology is based on the ability of the T-cells to regulate its activation cascade on interaction with the genetically modified antigen-presenting cell. The complex is formed by engineering two cell types that compose the reporter cell complex, a T-cell-based reporter cell and an antigen-presenting target cell.

The target cell is a fast-growing clone of an easy-to-transfect cell line derived from the kidney cells of a human embryo (HEK293T/17) that has been genetically engineered to stably display the Sgp of the SARS-CoV-2 virus. The sequence of Sgp can be exchanged to present any other viral antigenic biomarker. The reporter cell is a fast-growing, immortalized human T-acute lymphoblastic leukemia (T-ALL) cell line (Jurkat cells, doubling time around 20 hours) genetically engineered to upregulate a dual reporter system that exhibits fluorescence (green fluorescent protein, GFP) and bioluminescence (NanoLuc® or Nluc) on recognizing the Fc region of an Sgp-specific immunoglobulin G (IgG) bound to Sgp on the target cell.

Referring back to FIG. 6B, FIG. 6B illustrates a schematic of the reporter cell complex that detects the anti-Sgp IgG antibodies. The target cell is composed of HEK293T/17 as the cellular chassis engineered to stably express the viral Sgp on its surface. The Sgp insert was sub-cloned into the PiggyBac system backbone and was then used for stable integration to generate the genetically modified target cell (see FIG. 6C). This approach transforms the reporter cell complex serology test technology into a broadly applicable platform because the Sgp sequence can be exchanged for any antigenic biomarker on the target cell, thereby redirecting the specificity of the complex toward the antibody of interest. This strategy was used to develop the reporter cell complex with specificity for SARS-CoV-2 or SARS-CoV-1. FIG. 6D details the gene insert that was used with the lentivirus transduction process to generate the reporter cell with specificity toward the Fc region of IgG antibodies. The reporter cell is encoded with an artificial cell-signaling pathway with the following three domains: the receptor element, a transmembrane molecule that includes an extracellular Fc-region binding portion (senor or binding domain) at its distal end and mobilizes; the actuator element, the T-cell's activation machinery that upregulates the effector protein, a dual reporter system (GFP-2A-Nluc) upregulated when the antibody is also bound to the Sgp on the target cell through its Fab portion.

To illustrate the function of the antibody-specific reporter cell complex for detecting the presence of virus-specific antibodies and identifying individuals with antibodies against viral infections, the parental HEK293T/17 cell line was transformed into two different types of target cells: 1) SARS-CoV-1-Sgp-cells and 2) SARS-CoV-2-Sgp-cells, which constitutively and stably express the Sgp from SARS-CoV-1 and SARS-CoV-2, respectively. The reporter cell was engineered to express GFP and Nluc transgenes as activation-inducible reporters (linked through a self-cleavable peptide linker) to quantitatively respond to the intensity of the stimulus. The sensor portion (part of the receptor element) of the reporter cell comprises the sequence of the Fc-region-binding domain from the bacterial Protein A (zz-domain; GenBank: M74186). The formation of immune synapse, assisted via the analyte antibody (see schematic in the FIG. 6B), results in transcriptional regulation of the reporter system (effector element) through intracellular calcium rise for quantitative assessment of the infection. To demonstrate the functionality of the reporter cell complex and its ability to identify different antibody (IgG) types, parallel validation assays were conducted using IgG antibodies with specificity for both SARS-CoV-1 and SARS-CoV-2; and, the assay were incubation with the two different target cells, SARS-CoV-1-Sgp-cell and SARS-CoV-2-Sgp-cell, respectively. Isotype IgG against the envelop glycoprotein of the West Nile Virus (Anti-WNV IgG) was used as a negative control in all panels and at the concentration similar to that of the analyte antibody.

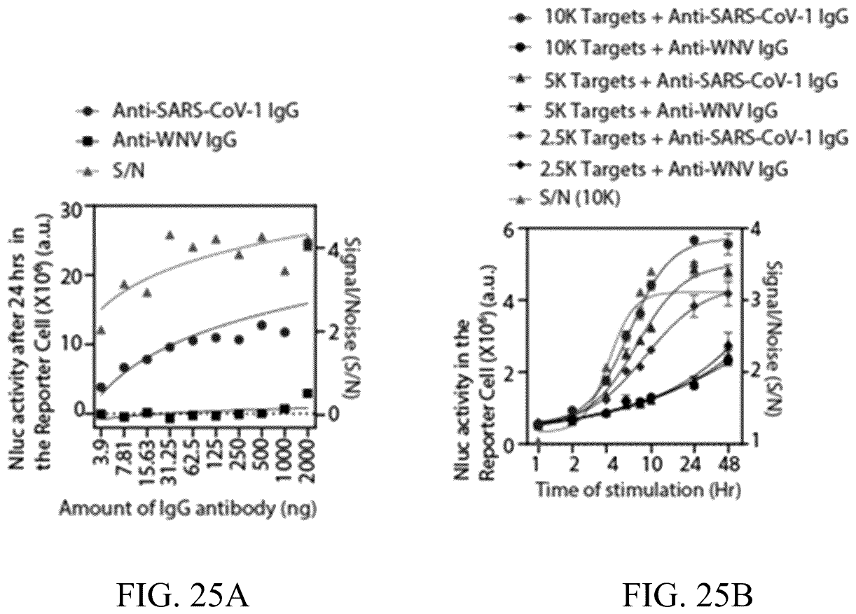

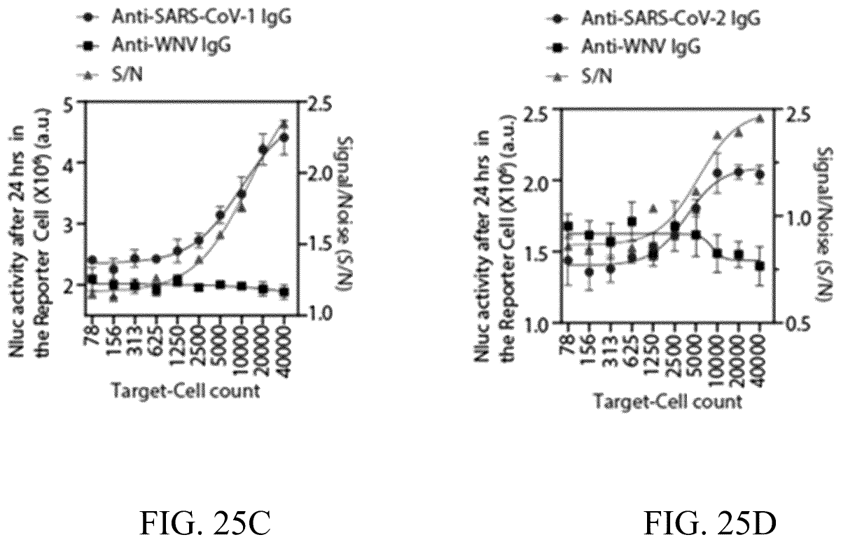

FIGS. 25A-25D illustrate plots characterizing assessment of example reporter cell complexes, in accordance with the present disclosure. A reporter cell was incubated with the target cell to form the reporter cell complex in presence of target cell specific IgG antibody. The Nluc activity in the reporter cell (12,500 cells) is proportional to the Anti-SARS-CoV-1 IgG antibody titer, as shown by FIG. 25A, increased with respect to time, as shown by FIG. 25B, increased proportionally to the number of engineered target SARS-CoV-1-Sgp-cells, as shown by FIG. 25C, and increased proportionally to the number of engineered target SARS-CoV-2-Sgp-cells, as shown by FIG. 25D. A total of 10,000 target cells were used for FIGS. 25A-25B and were varied in FIGS. 25C-25D as represented along the x-axis. In all experiments, anti-WVN-Egp antibody was used as a negative control. Nluc activity for all observations was measured using n=4; error bars indicate ±1 SD and can also be considered as one half the width of an 68% confidence interval for the mean.

FIG. 25A demonstrates that, in addition to the qualitative detection of the infection, the reporter (Nluc) activity of the reporter cell complex also provides a quantitative measure proportional to the antibody titer. The S/N, defined in the figure legend, quantifies the sensitivity of the reporter cell complex. The S/N was around 2 at low IgG antibody titer (around 4 ng) of the SARS-CoV-1-specific IgG (p<0.0001); the reporter cell-to-target cell ratio (R:T)=1.25:1 and logarithmically increases to around 4 at higher titers, a generally reported observation in serology tests. The data in FIG. 25B illustrates the reporter kinetics of the reporter cell complex upon engaging SARS-CoV-1-specific IgG and compared to the isotype control antibodies. The reporter cell complex, with serially diluted target cells (10,000; 5,000; and 2,500 SARS-CoV-1-Sgp-cells) was used. The Nluc activity from the reporter cell was detected as early as 2 hours (S/N=around 1.4, p<0.002) when a 10,000 target cells were used, and it increased steadily for the duration of the assay (at 48 hours, p<0.0001). The wide time window for the readout can assist mass screening efforts by accommodating a large test-sample size. A representative S/N curve is included for 10,000 SARS-CoV-1-Sgp-cells. Additionally, while the signal from the reporter cell was high in proportion to the higher target cell count, the kinetics of the test were faster at a lower target cell count. At 1 hour, a statistically significant increase in the signal was observed for 2,500 targets (S/N=around 1.16, p<0.005) and 5,000 targets (S/N=around 1.14, p<0.005), compared to 10,000 target cells (S/N=around 1.04, p=0.16).

FIGS. 25C-25D demonstrate the platform nature of the reporter cell complex and the effect of the target cell count on the sensitivity of the test. The data shows that, when using the same amount of target-specific IgG antibody, the reporter cell activity increases proportionately to the number of target cells. On the other hand, the signal from the reporter cell complex when incubated with the isotype-control antibody did not increase. FIG. 25C uses the SARS-CoV-1-Sgp-cells as target cells and detects SARS-CoV-1-specific IgG; the signal was statistically higher compared to isotope control (p<0.02 at all R:T<20:1). FIG. 25D uses the SARS-CoV-2-Sgp-cells to detect SARS-CoV-2-specific IgG; signal in this case was statistically higher at all R:T<1.25:1 (p<0.002), compared to the isotope control.

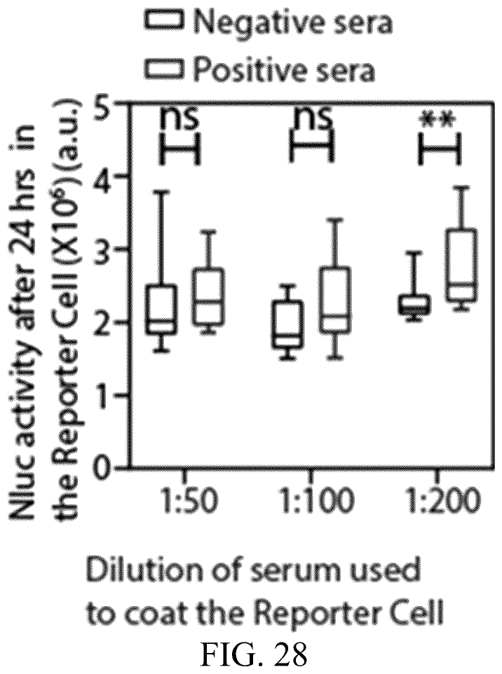

To characterize the performance of the serology test platform for detecting SARS-CoV-2 IgG antibodies, a commercially available panel of serum samples from 10 COVID-19 patients and 10 negative controls was used. All specimens were previously examined using the Abbott Architect IgG assay for SARS-CoV-2 IgG antibodies. Similar procedures, as reported in FIGS. 25A-25D, were employed to create the reporter cell complex, and the results were assessed for sensitivity and specificity. Patient sera was initially serially diluted (1:50, 1:100, 1:200) in the cell-culture media to determine the optimal dilution for improved sensitivity (FIG. 28). The dilution of 1:200 (p<0.05) was selected for use in subsequent assays and can be further optimized.

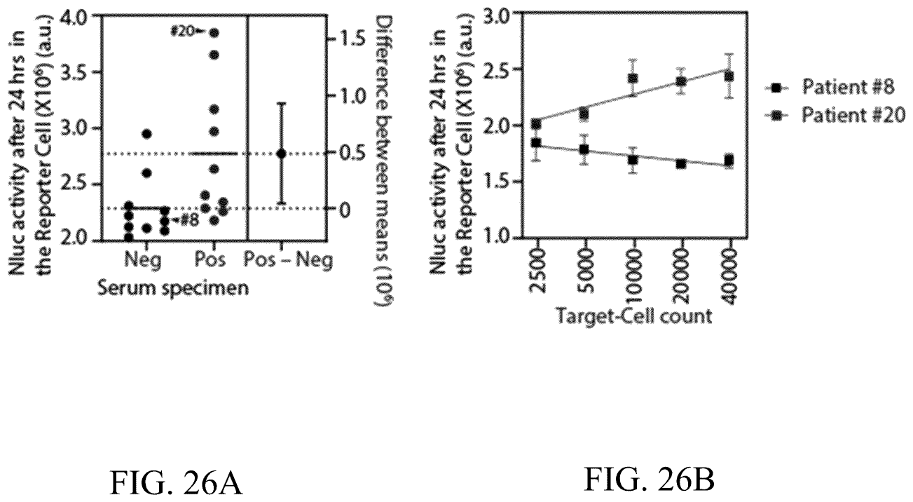

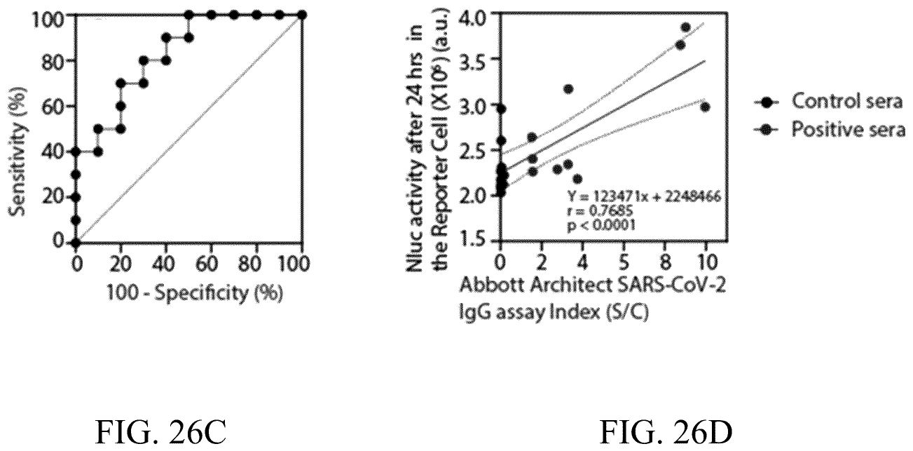

FIGS. 26A-26D illustrate plots characterizing assessment of use of an example reporter cell complex in a serology test using a commercial serum panel, in accordance with the present disclosure. FIG. 26A is an estimation plot that shows that the reporter cell complex significantly differentiates (p<0.05) between patient sera (positive, n=10) and control sera (negative, n=10) samples (1:200 sera dilution; 10,000 target SARS-CoV-2-Sgp-cells; 24 hours). The horizontal lines indicate the mean values for the respective groups. The p value was calculated using an unpaired two-tailed Student's t-test. FIG. 26B shows that the Nluc activity in the reporter cell (12,500 cells) increased proportionally to the target SARS-CoV-2-Sgp-cells (10,000 cells) with specimen #20 (positive) serum but not with specimen #8 (negative) serum. FIG. 26C shows an ROC analysis of the reporter cell complex, using the Abbott Architect IgG assay as the gold standard, with an AUC value of 0.830. FIG. 26D shows a correlation analysis of 20 SARS-CoV-2 sera panels with different levels of SARS-CoV-2 IgG antibodies analyzed by the Abbott Architect IgG assay verses the reporter cell complex. Correlation and linear regression analyses were performed using Pearson's correlation coefficients. Mean Nluc activity values of Diagnostic-Cell-Complex are plotted on the y-axis, while index values of the Abbott Architect IgG assay are shown on the x-axis. Statistical significance was calculated using the two-tailed test. The dashed lines indicate the standard deviations of the linear regression plots. Nluc activity for all observations was measured using n=4; error bars indicate ±1 SD.

The estimation plot in FIG. 26A demonstrates the magnitude of the difference in means of two clinical specimen groups (positive or negative for the SARS-CoV-2 antibodies) as assessed by a serology test. At the 95% confidence interval, the difference between the two means is greater than zero, demonstrating statistical significance (p<0.05) of the test. FIG. 26B illustrates an enhanced approach in context of the reporter cell complex, which was introduced to differentiate between the seropositive specimens at a low antibody titer from the seronegative specimens. It is believed that, unlike the seronegative specimen, incubation of the fixed quantity of seropositive specimen with serially diluted target cell count will proportionately reduce the stimulation of the reporter cell and show up as a slope of the reporter cell signal. This approach was confirmed as shown in FIG. 26B. The presence of slope in the reporter cell signal from the reporter cell complex, when incubated with a seropositive specimen differentiates it from the seronegative specimen (p<0.004 at all R:T<2.5:1) and offers to minimize the instances of false-negatives and false-positives. To determine the robustness of the serology test based on the reporter cell complex and its performance in clinical-sample testing, a receiver operating characteristics (ROC) curve analysis was used, as shown in FIG. 26C. Using the Abbott Architect IgG assay as the gold-standard test, the ROC curve showed an area-under-the-curve (AUC) value of 0.83 (p<0.05). At a cut-off value of 2276910 a.u., the serology assay showed an optimal sensitivity and specificity of 80% and 70%, respectively. Table 3 reports a complete analysis of the results, showing a positive predictive value of 72.7%, a negative predictive value of 77.8%, and a likelihood ratio of 2.667. FIG. 26D demonstrates the linear relationship between our reporter cell complex test benchmarked against the Abbott Architect IgG assay as the reference standard. Pearson-correlation analysis between results of the 20 serum samples using the reporter cell complex serology test and the gold standard produced a correlation coefficient of 0.77. Linear regression analysis indicated the R-squared value of 0.59 (p<0.0001) when reporter cell activity from the reporter cell complex-based serology test was plotted against the Abbott Architect IgG assay values.

TABLE 3
Contingency table analysis to determine the accuracy of the
Diagnostic-Cell-Complex, using the Wilson-Brown method;
Abbott Architect IgG assay used as the gold standard.

| | COVID-19 | COVID-19 | |
|---|---|---|---|
| | IgG | IgG | |
| Data analyzed | positive | negative | Total |
| Diagnostic-Cell-Complex | 8 | 3 | 11 |
| Positive | | | |
| Diagnostic-Cell-Complex | 2 | 7 | 9 |
| Negative | | | |
| Total | 10 | 10 | 20 |

| Effect size | Value | 95% CI |
|---|---|---|
| Sensitivity | 0.8 | 0.4902 to 0.9645 |
| Specificity | 0.7 | 0.3968 to 0.8922 |
| Positive Predictive Value | 0.7273 | 0.4344 to 0.9025 |
| Negative Predictive Value | 0.7778 | 0.4526 to 0.9605 |
| Likelihood Ratio | 2.667 | |

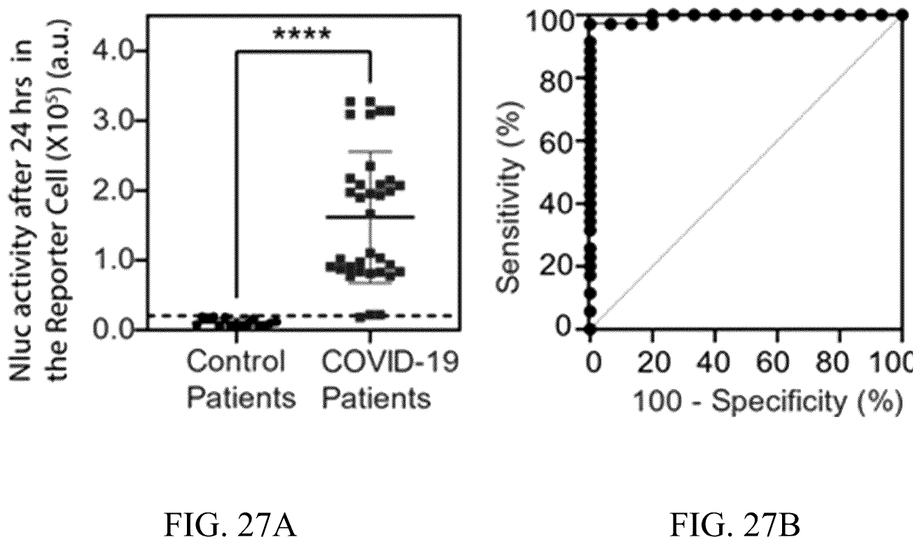

FIGS. 27A-27B illustrate plots characterizing clinical validation of an example reporter cell complex for detecting IgG antibodies specific to SARS-CoV-2, in accordance with the present disclosure. FIG. 27A is a scatter plot showing that the reporter cell complex significantly differentiates between control subjects and COVID-19 patients (p<0.0001). The dotted horizontal line represents a positive cut-off value based on the ROC analysis. The p value was calculated using an unpaired two-tailed student's t-test. FIG. 27B shows an ROC analysis of the reporter cell complex, based on qPCR results, with an AUC value of 0.9941. Patient sera was diluted at 1:200 and incubated with target SARS-CoV-2-Sgp-cells (10,000 cells) for 24 hours (COVID-19 patients, n=34 and control patients, n=15). Nluc activity for each serum sample was measured using n=4; error bars indicate ±1 SD of the mean.

To ensure reliability and reproducibility, FIGS. 27A-27B illustrates the results from the serology test conducted on another set of 49 serum samples (control subjects=15, COVID-19 patients=34) obtained from UC Davis Health. All serum samples used for validation were from patients with confirmed qPCR test results for SARS-CoV-2 RNA. FIG. 27A shows that the serology test detected Sgp-IgG antibodies in individuals with prior exposure to SARS-CoV-2 virus compared to the control subjects (p<0.0001). For the proof-of-principle studies, the signal from the reporter cell complex was assessed after 24 hours. The results in FIG. 27B show that the test can be significantly faster with the potential to inform on the seropositivity within 2 hours. Based on the clinical status (qPCR result), the ROC curve analysis in FIG. 27B further demonstrates the accuracy of the reporter cell complex serology assay with an AUC value of 0.9941 (p<0.0001). At a cut-off value of 18824 a.u., the serology assay showed an optimal sensitivity of 97.04% and specificity of 93.33% for detecting anti-Sgp IgG antibodies. Table 4 below shows a contingency-table analysis with a positive predictive value of 97.04%, a negative predictive value of 93.33%, and a likelihood ratio of 14.56.

TABLE 4
Contingency table analysis to determine the accuracy of the
reporter cell complex, using the Wilson-Brown method;
RT-qPCR used to determine clinical status of patients.

| | COVID-19 | COVID-19 | |
|---|---|---|---|
| Data analyzed | positive | negative | Total |
| Diagnostic-Cell-Complex | 33 | 1 | 34 |
| Positive | | | |
| Diagnostic-Cell-Complex | 1 | 14 | 15 |
| Negative | | | |
| Total | 34 | 15 | 49 |

| Effect size | Value | 95% CI |
|---|---|---|
| Sensitivity | 0.9706 | 0.8508 to 0.9985 |
| Specificity | 0.9333 | 0.7018 to 0.9966 |
| Positive Predictive Value | 0.9706 | 0.8508 to 0.9985 |
| Negative Predictive Value | 0.9333 | 0.7018 to 0.9966 |
| Likelihood Ratio | 14.56 | |

FIG. 28 illustrates a plot characterizing sensitivity of an example reporter cell complex using different sera dilutions, in accordance with the present disclosure. The Nluc activity in the reporter cell (12,500 cells) varied with respect to serum dilutions. SARS-CoV-2-Sgp-cells (10,000 cells) were used. Each data bar represents Nluc activity for observations measured using n=10; error bars indicate ±1 SD.

Synthesis and Experimental Information for Reporter Cell Complex

(1) Materials and reagents. Jurkat E6-1 (ATCC, Cat #TIB-152) cell line was maintained in complete RPMI media (RPMI1640 [Corning, Cat #10-040-CV], 10% heat-inactivated fetal bovine serum or FBS [Sigma-Aldrich, Cat #F2442-500ML], and 1× Penicillin-Streptomycin solution [Corning, Cat #30-002-Cl]). HEK293T/17 cells (ATCC, Cat #CRL-11268) cultured in complete DMEM (DMEM growth media [Corning, Cat #10-013-CV] supplemented with 10% heat-inactivated FBS and 1× Penicillin-Streptomycin). Plasmids encoding different genetic payloads (transfer plasmids) were designed in SnapGene software (GSL Biotech LLC) and sub-cloned into lentivirus vector plasmid (System Biosciences, Cat #CD510B-1). 2nd generation packaging plasmids (psPAX2 [Cat #12260] and pMD2.G [Cat #12259]) were obtained from Didier Trono (Ecole Polytechnique Federale de Lausanne, Lausanne, Switzerland) through Addgene. pAdvantage was obtained from Promega (Cat #E1711). Johns Hopkins University School of Medicine provided the PiggyBac Transposase sequence. All plasmid preparation services (chemical synthesis of DNA insert sequences, sub-cloning into respective vector backbones, and the amplification) were obtained from Epoch Life Science, Inc. (Missouri City, TX). For lentivirus production, plasmid transfections into parental HEK293T/17 cells were performed using Transporter 5™ reagent (Polysciences, Inc., Cat #26008-5). Polybrene (Abm®, Cat #G062) was used for lentivirus transductions into Jurkat cells. TransIT®-2020 transfection reagent (Mirus #MIR5400) was used to transfect and make stable target cells. Puromycin dihydrochloride (ThermoFisher Scientific, Cat #A1113803) was used for selecting stable cells. Anti-SARS-CoV-1 Sgp monoclonal antibody or S230 (Absolute Antibody, Cat #Ab00268), Anti-SARS-CoV-2 Sgp monoclonal antibody (SinoBiological, Cat #40150-R007), and Anti-West Nile virus envelop glycoprotein (WNV-Egp) monoclonal antibody (SinoBiological, Cat #40345-MM03) were used for characterization experiments of the reporter cell complex. Nano-Glo® assay kit (Promega, Cat #N1120) was used to assess expression of the Nluc protein.

(1.1) Source of COVID-19 patient sera samples. For the initial assay characterization, a commercially available panel of 20 COVID patient serum specimens (10 positive SARS-CoV-2-IgG and 10 negative SARS-CoV-2-IgG sera samples), confirmed using the Abbott Architect SARS-CoV-2-IgG assay, were obtained from Boca Biolistics (Cat #C0050-0001). The samples were heat-inactivated at 65° C. for 30 minutes to allow safe handling in a BSL2 laboratory, and stored in a −80° C. freezer until required. For clinical validation, 49 serum samples from individuals with confirmed qPCR results for presence/absence of SARS-CoV-2 RNA were obtained from UC Davis Health (University of California Davis) (control subjects, n=15 and COVID-19 confirmed patients, n=34).

(1.2) Lentivirus production. Lentivirus particles were prepared by packaging the transfer plasmid using 2^{nd} generation lentivirus system as detailed previously.

(1.3) Generation of the reporter cell (R) component of the reporter cell complex. The Jurkat E6-1 suspension cell line was engineered with lentivirus particles carrying the genetic payload (FIG. 6B), as detailed previously. The receptor domain was replaced with the synthetic sequence from the bacterial Protein A (zz-domain; GenBank: M74186), previously reported to bind to the Fc region of immunoglobulin G (IgG) antibodies. The cells were treated with lentivirus in the presence of 8 μg/mL Polybrene. After 48 hours, the engineered cells were placed in selection using 0.5 μg/ml of puromycin dihydrochloride. The unmodified parental cell line was also placed under selection as a positive control for determining the minimum lethal concentration of puromycin. Following selection, cells were expanded as required for different assays and frozen using freezing media.

(1.4) Generation of the target cell (T) component of the reporter cell complex. The HEK293T/17 adherent cells were engineered to stably express viral Sgp, using the PiggyBac Transposon system, as previously described. Two plasmids were designed with the piggyBac transposon backbone (System Biosciences, Cat #PB510B-1) to either express the Sgp of SARS-CoV-1 (SARS-CoV-1-Sgp; GenBank: AAP13567.1) or of SARS-CoV-2 (SARS-CoV-2-Sgp; GenBank: QHD43416.1) on the cell surface. A monolayer of HEK293T/17 cells were transfected with both the Transposon plasmid (carrying gene of interest) and Transposase plasmid, in a ratio of 2.5:1, using TransIT®-2020 transfection reagent. After 48 hours of transfection, the transfected cells were placed under selection using Puromycin dihydrochloride. The unmodified parental HEK293T/17 cell line was also placed under selection as a positive control to determine the minimum lethal concentration of puromycin. The generated stable cell lines were labeled as SARS-CoV-1-Sgp-cells (HEK293T/17 cells engineered to stably express the Sgp from SARS-CoV-1) and SARS-CoV-2-Sgp-cells (HEK293T/17 cells engineered to stably express the Sgp from SARS-CoV-2). The cells were then expanded for different assays.

(1.5) Method of use of reporter cell complex for serology test.

(i) With research-grade antibodies against SARS-CoV-1-Sgp or SARS-CoV-1-Sgp during technology development (FIGS. 25A-25D and 28). The reporter cell (12,500 in 50 μL of complete RPMI media) was incubated with varying concentrations (or as stated) of the Anti-SARS-CoV-1 IgG (S230) or Anti-SARS-CoV-2 IgG monoclonal antibodies for 30 minutes at 37° C., washed once, and then co-cultured with 10,000 target cells (SARS-CoV-1-Sgp-cells or SARS-CoV-2-Sgp-cells, respectively) in 100 μL of complete RPMI media per well of a 96-well plate. As a negative control, the reporter cell was coated using a non-specific Anti-WNV-Egp monoclonal antibody and co-cultured with the same target cells. After the specified time in co-culture, NanoLuc® (Nluc) activity in the reporter cell was assessed using Nano-Glo® assay kit, following the manufacturer's instructions. The Nluc enzyme substrate was diluted in the cell-lysis buffer provided with the Nano-Glo® and added to the co-cultures in 96-well plate for assessing enzyme activity. Following a brief incubation period of 3 minutes, bioluminescence was read on a microplate reader (Perkin Elmer, Envision™ Multilabel Plate Reader Model: 2104-0010A).

(ii) With COVID-19 patient serum (FIGS. 26A-26D and 27A-27B). For each serum sample, the reporter cell (12,500 in 50 μL of complete RPMI media) was incubated with the respective patient serum at different dilutions (or as stated) for 30 minutes at 37° C., washed once and then co-cultured with 10,000 (or varying numbers) of target SARS-CoV-2-Sgp-cells, in 100 μL of complete RPMI media per well of a 96-well plate. After the 24 hours in co-culture, NanoLuc® (Nluc) activity in the reporter cell was assessed using Nano-Glo® assay as described earlier.

(1.6) Statistical analysis. The experimental design for each panel in the figures is described below. GraphPad Prism 9.2.0 (GraphPad Software, Inc.) was used to conduct all statistical analysis.

(i) FIGS. 25A-25D (Development of method for using the reporter cell complex). Statistical analyses were based on multiple t-tests using a two-stage linear step-up procedure of Benjamini, Krieger, and Yekutieli with a FDR of 1%. There was no adjustment for multiple comparisons. The S/N is calculated as the ratio of the mean Nluc activity in the reporter cell when the reporter cell complex is incubated with Anti-SARS-CoV-1-Sgp-IgG or Anti-SARS-CoV-2-Sgp-IgG antibodies, divided by the mean Nluc activity in the reporter cell when the reporter cell complex is incubated with Anti-WNV-Egp-IgG antibodies. The error bars extend 1 SD above and below the mean and can also be considered as one half the width of a 68% confidence interval for the mean.

(ii) FIG. 25A (Nluc activity in the reporter cell is proportional to the concentration of antibodies). The Nluc activity in the reporter cell when stimulated by the target SARS-CoV-1-Sgp-cells was fitted using a semi-log logistic model Y=a+b*log 10(X), where X is the concentration (ng) of IgG antibodies used to coat the reporter cell, a=Y-intercept, and b=Slope.

(iii) FIG. 25B (Nluc activity in the reporter cell is a function of duration of stimulation). The Nluc activity in the Anti-SARS-CoV-1-Sgp-coated (or Anti-WNV-Egp-coated) reporter cell stimulated by the target cells (SARS-CoV-1-Sgp-cells) was fitted using a four-parameter logistic model Nluc=Nlucₘᵢₙ+{Nlucₘₐₓ−Nlucₘᵢₙ}/{1+10\hat{ }[b*(log₁₀[Time₅₀]−X)]}; where X is the log₁₀ of the duration of activation (hours) by the target cells, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and Time₅₀ is an estimated parameter representing the X value corresponding to (Nlucₘₐₓ−Nlucₘᵢₙ)/2.

(iv) FIGS. 25C-25D (Nluc activity in the reporter cell is proportional to the target cell count). The Nluc activity in the reporter cell when stimulated by the target SARS-CoV-1-Sgp-cells or SARS-CoV-2-Sgp-cells was fitted using a four-parameter logistic model Nluc=Nlucₘᵢₙ+{Nlucₘₐₓ−Nlucₘᵢₙ}/{1+10\hat{ }[b*(log₁₀[Target₅₀]−X)]}; where X is the log₁₀ of the target cell count, Nlucₘₐₓ is an estimated parameter defining a upper asymptote for Nluc activity, Nlucₘᵢₙ is an estimated parameter defining a lower asymptote for Nluc activity, b is a “Hill” parameter defining the slope at the inflection point of the fitted curve, and Target₅₀ is an estimated parameter representing the X value corresponding to (Nlucₘₐₓ−Nlucₘᵢₙ)/2. FIG. 25C shows results with Anti-SARS-CoV-1-IgG (S230) while FIG. 25D shows results with Anti-SARS-CoV-2-IgG (Anti-WNV-IgG as negative control in both experiments).

(v) FIGS. 26A-26D (Characterization of the reporter cell complex for serology test using a commercial serum panel). Statistical analysis was based on an unpaired two-tailed student's t-test with common variance and the p-value of <0.05 was considered statistically significant. Statistical analysis for FIG. 26B was based on multiple t-tests using a two-stage linear step-up procedure of Benjamini, Krieger, and Yekutieli with a FDR of 1%. The error bars extend 1 SD above and below the mean and can also be considered as one half the width of a 68% confidence interval for the mean.

(vi) FIG. 26A (Reporter cell complex differentiates between positive patient sera and negative sera samples). An estimation plot (confidence interval of 95%) shows the difference between positive patient sera and negative sera samples, using an unpaired two-tailed student's t-test, assuming Gaussian distribution and that both sample means have the same standard deviations.

(vii) FIG. 26B (Specificity of the reporter cell complex in differentiating sera samples with varying IgG antibody titers). The Nluc activity in the serum-coated reporter cell stimulated by the target SARS-CoV-2-Sgp-cells was fitted using a straight-line equation Y=a+b*log₁₀(X); where X is the target cell count used to activate the reporter cell, a=Y-intercept, and b=Slope.

(viii) FIG. 26C (ROC curve analysis). The ROC curve analysis was performed using the Wilson/Brown method at 95% Confidence Interval, and results on the ROC curve are expressed as percentages. Abbott Architect SARS-CoV-2-IgG assay was used to classify patient samples.

(ix) FIG. 26D (Correlation between the reporter cell complex serology test and the gold standard test). Simple linear regression was used to plot the goodness of fit between the two assays and determine the R2 statistic, while the nonparametric Pearson-Spearman method was used to determine the correlation coefficient, R. A two-tailed p-value was used to determine if the correlation between the two assays is significant.

(x) FIG. 27A (Clinical validation of the reporter cell complex for detecting COVID-19 IgG antibodies in patients). A scatterplot shows the difference between COVID-19 patient sera and control sera samples, using an unpaired two-tailed student's t-test, assuming a Gaussian distribution with a Welch's correction since standard deviations are not assumed equal.

(xi) FIG. 27B (ROC curve analysis). The ROC curve analysis was performed using the Wilson/Brown method at 95% Confidence Interval, and results on the ROC curve are expressed as percentages. RT-qPCR results were used to determine clinical status and classify patient serum.

(xii) FIG. 28 (Sensitivity of the reporter cell complex while using serially diluted sera). The difference between the multiple box plots was determined using the Holm-Sidak method, assuming that all sample groups had similar standard deviations.

(xiii) Table 3 and Table 4 (Contingency table analysis for human COVID-19 sera samples). Confidence intervals for the specificities, sensitivities, and predictive values were calculated using the Wilson/Brown method. The p-values were calculated using the Fisher's exact t-test. Table 3 shows results using a commercial serum panel while Table 4 shows results using clinical samples from UC Davis Health.

Table 5 below provides a list of genes used to engineer example reporter cells and target cells.

TABLE 5
List of genes used to engineer the reporter
cells and target cells

| GENETIC ELEMENT | SEQ ID NO |
|---|---|
| SARS-CoV-2 Sgp | SEQ ID NO: 1 |
| SARS-CoV-1 Sgp | SEQ ID NO: 2 |
| ZZ domain (Fc-region-binding | SEQ ID NO: 55 |
| domain from the bacterial Protein A) | |
| Luc2 | SEQ ID NO: 8 |
| E2 Crimson | SEQ ID NO: 9 |
| Luc2-P2a-E2 Crimson | SEQ ID NO: 10 |
| GFP | SEQ ID NO: 11 |
| Nluc | SEQ ID NO: 12 |
| P2a | SEQ ID NO: 13 |
| GFP-P2a-Nluc | SEQ ID NO: 14 |
| Plasmid 2 (SARS-CoV-2 Sgp) | SEQ ID NO: 16 |
| Plasmid 3 (SARS-CoV-1 Sgp) | SEQ ID NO: 17 |
| Plasmid 26 (ZZ domain) | SEQ ID NO: 56 |

Various embodiments are implemented in accordance with the underlying Provisional Applications: Ser. No. 63/142,315, entitled “SARS-CoV-2 Specific Therapeutic Cell Biofactory,” filed Jan. 27, 2021; Ser. No. 63/222,784, entitled “Diagnostic-Cell Platform for Antigen Testing,” filed Jul. 16, 2021; and Ser. No. 63/255,380, entitled “Cell-based Serological Test for Covid-19,” filed Oct. 13, 2021, to which benefit is claimed and which are fully incorporated herein by reference for their general and specific teachings. For instance, embodiments herein and/or in the Provisional Applications can be combined in varying degrees (including wholly). Reference can also be made to the experimental teachings and underlying references provided in the underlying Provisional Applications. Further embodiments are implemented in accordance with the underlying US Application U.S. Ser. No. 15/263,078, entitled “Genetically Engineered Cells as a Modular Platform for Cell-based Medicine”, filed on Sep. 12, 2016, to which benefit is claimed, as a continuation-in-part, and which is fully incorporated herein by reference for its general and specific teachings. Embodiments discussed in the Provisional Applications are not intended, in any way, to be limiting to the overall technical disclosure, or to any part of the claimed disclosure unless specifically noted. In some embodiments, the genetically modified effector cells, diagnostic cells, and/or reporter cells can include at least some of the components or features as described by Repellin C E, et al., entitled “Modular Antigen-Specific T-cell Biofactories for Calibrated In Vivo Synthesis of Engineered Proteins”, Advanced Biosystems, 2(12):1800210 (2018), and Repellin C E, et al, entitled “NK-Cell Biofactory as an Off-the-Shelf Cell-based Vector for Targeted In Situ Synthesis of Engineered Proteins”, Advanced BioSystems 5(7): 2000398 (2021), each of which are hereby incorporated in their entirety for their teaching.

Although specific embodiments have been illustrated and described herein, a variety of alternate and/or equivalent implementations can be substituted for the specific examples shown and described without departing from the scope of the present disclosure. This application is intended to cover any adaptations or variations of the specific embodiments and examples discussed herein.

1. An antigen test device, comprising:
genetically engineered diagnostic cell comprising an exogenous polynucleotide sequence that includes, in operative association:
a receptor element that encodes a chimeric antigen receptor (CAR) comprising an extracellular antigen binding domain operably linked to a transmembrane domain, and an intracellular signaling domain, wherein the extracellular antigen binding domain recognizes an antigen on a surface of a pathogen-infected cell from a sample or on a surface of a virus particle associated with a pathogen from the sample;
an actuator element that encodes a transcription factor binding site that upregulates synthesis of a detectable reporter protein in response to the antigen binding domain of the CAR binding to the antigen; and
an effector element that encodes the detectable reporter protein, wherein, in response to the antigen binding domain of the CAR binding to the antigen, the genetically engineered diagnostic cell is configured to activate and, to synthesize the detectable reporter protein which indicates infection of the sample by the pathogen.

2. The antigen test device of claim 1, wherein an amount of the detectable reporter protein is proportional to the amount of the antigen present in the sample.

3. The antigen test device of claim 1, further comprising a solution containing the genetically engineered diagnostic cell, wherein the solution is further configured to contain the sample.

4. The antigen test device of claim 1, wherein the extracellular antigen binding domain is configured to recognize both the antigen on the surface of the pathogen-infected cell and the antigen on the surface of the virus particle.

5. The antigen test device of claim 1, wherein the detectable reporter protein comprises a first detectable reporter protein and a second detectable reporter protein linked by a 2A linker peptide.

6. The antigen test device of claim 5, wherein the first and second detectable reporter proteins are each selected from a fluorescent protein and luciferase.

7. The antigen test device of claim 1, wherein the effector element further encodes a signal peptide that is non-native to the detectable reporter protein.

8. The antigen test device of claim 1, wherein the pathogen is Severe Acute Respiratory Syndrome Coronavirus 2 (SARS-CoV-2), Severe Acute Respiratory Syndrome Coronavirus 1 (SARS-CoV-1), Ebola, Marburg, Chikungunya, Nipah, or West Nile.

9. The antigen test device of claim 1, wherein the extracellular antigen binding domain comprises a single-domain heavy chain (VHH) of an antibody that binds to the antigen.

10. The antigen test device of claim 1, wherein the antigen test device comprises a panel configured to test for a plurality of different pathogens including the pathogen, the panel comprising a plurality of the genetically engineered diagnostic cell having extracellular antigen binding domains that respectively recognize an antigen on a surface of a respectively different pathogen-infected cell or virus particle from the sample.

11. A genetically engineered diagnostic cell comprising an exogenous polynucleotide sequence that includes, in operative association:
a receptor element that encodes a chimeric antigen receptor (CAR) comprising an extracellular antigen binding domain operably linked to a transmembrane domain, and an intracellular signaling domain, wherein the extracellular antigen binding domain recognizes an antigen on a surface of a pathogen-infected cell from a sample or on a surface of a virus particle associated with a pathogen from the sample;
an actuator element that encodes a transcription factor binding site that upregulates synthesis of a dual-reporter system in response to the antigen binding domain of the CAR binding to the antigen; and
an effector element that encodes the dual-reporter system, wherein, in response to the antigen binding domain of the CAR binding to the antigen, the genetically engineered diagnostic cell is configured to activate and to synthesize the dual-reporter system and express signals indicative of infection of the sample by the pathogen.

12. The genetically engineered diagnostic cell of claim 11, wherein the dual-reporter system comprises a first detectable reporter protein and a second detectable reporter proteins coupled by a linker peptide.

13. The genetically engineered diagnostic cell of claim 11, wherein the dual-reporter system comprises a fluorescent protein and a bioluminescent protein coupled by a self-cleaving linker peptide.

14. The genetically engineered diagnostic cell of claim 11, wherein the extracellular antigen binding domain comprises a single-domain heavy chain (VHH) region of an antibody specific to the antigen.

15. The genetically engineered diagnostic cell of claim 11, wherein an intensity of the signals expressed is proportional to an amount of the antigen present in the sample and is indicative of disease burden.

16. The genetically engineered diagnostic cell of claim 15, wherein the signals comprise fluorescence and bioluminescent signals, and the extracellular antigen binding domain is configured to recognize both the antigen on the surface of the pathogen-infected cell and the antigen on the surface of the virus particle.

17. A method, comprising:
contact a sample with a solution containing at least a portion of plurality of genetically engineered diagnostic cells, wherein the at portion of the plurality of genetically engineered diagnostic cells comprise:
a receptor element that encodes a chimeric antigen receptor (CAR) comprising an extracellular antigen binding domain operably linked to a transmembrane domain, and an intracellular signaling domain, wherein the extracellular antigen binding domain recognizes an antigen on a surface of a pathogen-infected cell from a sample or on a surface of a virus particle associated with a pathogen from the sample;
an actuator element that encodes a transcription factor binding site; and
an effector element that encodes a dual-reporter system;
in response to contacting the sample with the solution and a presence of the pathogen-infected cell or virus particle in the sample, causing binding of the antigen binding domain to the antigen of the pathogen-infected cell or virus particle;
in response to the antigen binding domain of the CAR binding to the antigen, synthesizing the dual-reporter system and expressing signals indicative of infection of the sample by the pathogen; and
detecting an intensity of the signals, which is proportional to an amount of the antigen present in the sample and indicative of disease burden.

18. The method of claim 17, wherein the dual-reporter system is synthesized to express fluorescence and bioluminescent signals.

19. The method of claim 17, wherein the dual-reporter system comprises a fluorescent protein linked to a bioluminescent protein by a 2A linker peptide.

20. The method of claim 17, wherein other respective ones of the plurality of genetically engineered diagnostic cells are targeted to a plurality of different pathogens and to recognize different antigens on a surface of plurality of different pathogen-infected cells or virus particles from the sample, with the respective ones of the plurality of genetically engineered diagnostic cells are contain in the solution or additional solutions.
